(19) United States
(12) Patent Application Publication
(10) Pub. No.: US 2025/0180543 A1
(43) Pub. Date: Jun. 5, 2025

(54) COMPOSITIONS AND METHODS OF A HIGH-THROUGHPUT SCREEN TO IDENTIFY COMPOUNDS THAT ALTER MACROPHAGE-MEDIATED CYTOTOXICITY OF TARGET CELLS

(71) Applicants: Whitehead Institute for Biomedical Research, Cambridge, MA (US); Dana-Farber Cancer Institute, Inc., Boston, MA (US)

(72) Inventors: Kipp Weiskopf, Wellesley, MA (US); Kyle Vaccaro, Marlborough, MA (US); Juliet Allen, Pleasanton, CA (US); Asaf Maoz, Chestnut Hill, MA (US)

(73) Assignees: Whitehead Institute for Biomedical Research, Cambridge, MA (US); Dana-Farber Cancer Institute, Inc., Boston, MA (US)

(21) Appl. No.: 18/844,905

(22) PCT Filed: Mar. 8, 2023
(86) PCT No.: PCT/US2023/014836

§ 371 Date: Sep. 6, 2024

Related U.S. Application Data
(60) Provisional application No. 63/329,644, filed on Apr. 11, 2022.
(60) Provisional application No. 63/317,888, filed on Mar. 8, 2022.

(51) Int. Cl. G01N 33/50

(52) U.S. Cl. CPC G01N 33/5011; G01N 33/5055

(57) ABSTRACT

The present disclosure provides methods and compositions related to identifying agents that modulate macrophage function. The agents identified may be useful alone or in combination for treating and/or preventing diseases related to macrophage function (e.g., cancer) in a subject.

### RELATED APPLICATIONS

This patent application claims priority under 35 U.S.C. § 119 (e) to U.S. Provisional Applications, U.S. Ser. No. 63/317,888, filed Mar. 8, 2022, and U.S. Ser. No. 63/329,644, filed Apr. 11, 2022. The entire content of each is incorporated herein by reference.

### BACKGROUND

Macrophages are a type of white blood cell of the immune system that engulfs and digests pathogens, such as cancer cells, microbes, cellular debris, and foreign substances. which do not have proteins that are specific to healthy body cells on their surface. The process is called phagocytosis, which acts to defend the host against infection and injury.

Macrophages patrol for potential pathogens in essentially all tissues. They take various forms and names throughout the body (e.g., histiocytes, Kupffer cells, alveolar macrophages, microglia, and others). Besides phagocytosis, macrophages may also kill cells by mechanisms of direct cytotoxicity and trogocytosis. They play a key role in nonspecific defense (innate immunity) and also help initiate specific defense mechanisms (adaptive immunity) by recruiting other immune cells such as lymphocytes. For example, they are important as antigen presenters to T cells. In humans, dysfunctional macrophages cause severe diseases such as chronic granulomatous disease that result in frequent infections.

Beyond increasing inflammation and stimulating the immune system, macrophages also play an important anti-inflammatory role and can decrease immune reactions through the release of cytokines. Macrophages that encourage inflammation are called M1 macrophages, whereas those that decrease inflammation and encourage tissue repair are called M2 macrophages.

Macrophages play a role in a wide variety of important processes of the immune system, including wound healing, muscle regeneration, limb regeneration, iron homeostasis. pigment retainment, tissue homeostasis, and nerve function.

Macrophages are also involved in the pathogenesis of many diseases, including infectious diseases, cardiovascular disease, metabolic disorders, and cancer. Regarding cancer, macrophages within the tumor microenvironment may contribute to resistance and progression of certain tumor types (e.g., EGFR-mutant tumors). Macrophage infiltration into tumors (e.g., lung tumors) correlates with worse prognosis, and therapies that activate macrophages are emerging in cancer therapy. For example, one therapeutic target is the CD47-SIRPα interaction, which acts as a myeloid immune checkpoint. Cluster of Differentiation 47 (CD47) is highly expressed on many different types of cancer, including lung cancer. CD47 binds to an inhibitory receptor, signal-regulatory protein alpha (SIRPα), that is expressed on the surface of macrophages and other myeloid immune cells. When CD47 binds to SIRPα, it sends inhibitory signals to macrophages that prevent phagocytosis. CD47-blocking therapies stimulate macrophage phagocytosis of cancer cells and are effective across many preclinical cancer models. They have demonstrated efficacy in clinical trials for relapsed/refractory lymphoma and are under investigation for other solid and hematologic malignancies.

### SUMMARY

The present disclosure stems from the recognition that due to their ubiquitinous nature in processes related to the immune system and disease, there is a need to efficiently and effectively screen agents that modulate macrophage activity (e.g., activate or inhibit macrophage function) to identify therapeutic agents for a wide variety of disease.

In one aspect, the present disclosure provides a method of identifying an agent that modulates macrophage function, the method comprising:
providing a population of macrophages and a population of target cells in an assay mixture;
adding a candidate agent to the assay mixture;
culturing the assay mixture;
determining the amount of growth of the population of target cells in the assay mixture at one or more timepoints; and
comparing the amount of growth of the population of target cells in the assay mixture with the amount of growth of a population of the same type of target cells in a control mixture cultured without the candidate agent at the same timepoint;
wherein the candidate agent is determined to be an agent that modulates macrophage function when the amount of growth of the population of target cells in the assay mixture is different from the amount of growth of the population of target cells in the control mixture at the same timepoint.

In another aspect, the present disclosure provides a method of identifying an agent that promotes macrophage-mediated cytotoxicity, the method comprising:
providing a population of macrophages and a population of target cells in an assay mixture;
adding a candidate agent to the assay mixture;
culturing the assay mixture;
determining the amount of growth of the population of target cells in the assay mixture at one or more timepoints; and
comparing the amount of growth of the population of target cells in the assay mixture with the amount of growth of a population of the same type of target cells in a control mixture cultured without the candidate agent, at the same timepoint;
wherein the candidate agent is determined to be an agent that promotes macrophage-mediated cytotoxicity when the amount of growth of the population of target cells in the assay mixture is less than the amount of growth of the population of target cells in the control mixture at the same timepoint.

In another aspect, the present disclosure provides a method of identifying an agent that promotes macrophage anti-cancer function, the method comprising:
providing a population of macrophages and a population of target cells in an assay mixture;
adding a candidate agent to the assay mixture;
culturing the assay mixture;
determining the amount of growth of the population of target cells in the assay mixture at one or more timepoints; and
comparing the amount of growth of the population of target cells in the assay mixture with the amount of growth of a population of the same type of target cells in a control mixture cultured without the candidate agent at the same timepoint;
wherein the candidate agent is determined to be an agent that promotes macrophage anti-cancer function when the amount of growth of the population of target cancer cells in the assay mixture is lower than the amount of growth of the population of target cancer cells in the control mixture at the same timepoint.

In another aspect, the present disclosure provides a method of identifying an agent that selectively modulates macrophage function for a target cell, the method comprising:
providing a population of macrophages, a population of first target cells, and a population of second target cells in an assay mixture, wherein the first and second target cells are different;
adding a candidate agent to the assay mixture;
culturing the assay mixture;
determining the amount of growth of the populations of first and second target cells in the assay mixture at one or more timepoints;
comparing the amount of growth of the population of first target cells in the assay mixture with the amount of growth of a population of the first target cells in a control mixture cultured without the candidate agent, at the same timepoint;
comparing the amount of growth of the population of second target cells in the assay mixture with the amount of growth of a population of the second target cells in a control mixture cultured without the candidate agent, at the same timepoint;
wherein the candidate agent is determined to be an agent that selectively modulates macrophage function for a target cell when the amount of growth of the population of first target cells in the assay mixture compared to the amount of growth in the control mixture is different than the amount of growth of the population of second target cells in the assay mixture compared to the amount of growth in the control mixture at the same timepoint.

In another aspect, disclosed is an agent identified by any of the methods described herein.

### DEFINITIONS

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al., describe pharmaceutically acceptable salts in detail in J. Pharmaceutical Sciences, 1977, 66, 1-19, incorporated herein by reference. Pharmaceutically acceptable salts of the compounds described herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid, and perchloric acid or with organic acids such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄ alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “solvate” refers to forms of the compound, or a salt thereof, that are associated with a solvent, usually by a solvolysis reaction. This physical association may include hydrogen bonding. Conventional solvents include water, methanol, ethanol, acetic acid, DMSO, THF, diethyl ether, and the like. The compounds described herein may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolatable solvates. Representative solvates include hydrates, ethanolates, and methanolates.

The term “hydrate” refers to a compound that is associated with water. Typically, the number of the water molecules contained in a hydrate of a compound is in a definite ratio to the number of the compound molecules in the hydrate. Therefore, a hydrate of a compound may be represented, for example, by the general formula R·x H₂O, wherein R is the compound, and x is a number greater than 0. A given compound may form more than one type of hydrate, including, e.g., monohydrates (x is 1), lower hydrates (x is a number greater than 0 and smaller than 1, e.g., hemihydrates (R·0.5 H₂O)), and polyhydrates (x is a number greater than 1, e.g., dihydrates (R·2 H₂O) and hexahydrates (R·6 H₂O)).

The term “tautomers” or “tautomeric” refers to two or more interconvertable compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction interconverting a tautomeric pair) may be catalyzed by acid or base. Exemplary tautomerizations include keto-to-enol, amide-to-imide, lactam-to-lactim, enamine-to-imine, and enamine-to-(a different enamine) tautomerizations.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The term “polymorphs” refers to a crystalline form of a compound (or a salt, hydrate, or solvate thereof). All polymorphs have the same elemental composition. Different crystalline forms usually have different X-ray diffraction patterns, infrared spectra, melting points, density, hardness, crystal shape, optical and electrical properties, stability, and solubility. Recrystallization solvent, rate of crystallization, storage temperature, and other factors may cause one crystal form to dominate. Various polymorphs of a compound can be prepared by crystallization under different conditions.

The term “prodrugs” refers to compounds that have cleavable groups and become by solvolysis or under physiological conditions the compounds described herein, which are pharmaceutically active in vivo. Such examples include, but are not limited to, choline ester derivatives and the like. N-alkylmorpholine esters and the like. Other derivatives of the compounds described herein have activity in both their acid and acid derivative forms, but in the acid sensitive form often offer advantages of solubility, tissue compatibility, or delayed release in the mammalian organism (see, Bundgard, H., Design of Prodrugs, pp. 7-9, 21-24, Elsevier, Amsterdam 1985). Prodrugs include acid derivatives well known to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides, and anhydrides derived from acidic groups pendant on the compounds described herein are particular prodrugs. In some cases it is desirable to prepare double ester type prodrugs such as (acyloxy) alkyl esters or ((alkoxycarbonyl) oxy) alkylesters. C₁-C₈ alkyl, C₂-C₈ alkenyl, C₂-C₈ alkynyl, aryl, C₇-C₁₂ substituted aryl, and C₇-C₁₂ arylalkyl esters of the compounds described herein may be preferred.

The term “small molecule” refers to molecules, whether naturally-occurring or artificially created (e.g., via chemical synthesis) that have a relatively low molecular weight. Typically, a small molecule is an organic compound (i.e., it contains carbon). The small molecule may contain multiple carbon-carbon bonds, stereocenters, and other functional groups (e.g., amines, hydroxyl, carbonyls, and heterocyclic rings, etc.). In certain embodiments, the molecular weight of a small molecule is not more than about 1.000 g/mol. not more than about 900 g/mol, not more than about 800 g/mol, not more than about 700 g/mol, not more than about 600 g/mol, not more than about 500 g/mol, not more than about 400 g/mol, not more than about 300 g/mol, not more than about 200 g/mol, or not more than about 100 g/mol. In certain embodiments, the molecular weight of a small molecule is at least about 100 g/mol, at least about 200 g/mol, at least about 300 g/mol, at least about 400 g/mol, at least about 500 g/mol, at least about 600 g/mol, at least about 700 g/mol, at least about 800 g/mol, or at least about 900 g/mol, or at least about 1,000 g/mol. Combinations of the above ranges (e.g., at least about 200 g/mol and not more than about 500 g/mol) are also possible. In certain embodiments, the small molecule is a therapeutically active agent such as a drug (e.g., a molecule approved by the U.S. Food and Drug Administration as provided in the Code of Federal Regulations (C.F.R.)). The small molecule may also be complexed with one or more metal atoms and/or metal ions. In this instance, the small molecule is also referred to as a “small organometallic molecule.” Preferred small molecules are biologically active in that they produce a biological effect in animals, preferably mammals, more preferably humans. Small molecules include, but are not limited to, radionuclides and imaging agents. In certain embodiments, the small molecule is a drug. Preferably, though not necessarily, the drug is one that has already been deemed safe and effective for use in humans or animals by the appropriate governmental agency or regulatory body. For example, drugs approved for human use are listed by the FDA under 21 C.F.R. §§ 330.5, 331 through 361, and 440 through 460, incorporated herein by reference; drugs for veterinary use are listed by the FDA under 21 C.F.R. §§ 500 through 589, incorporated herein by reference. All listed drugs are considered acceptable for use in accordance with the present disclosure.

A “protein,” “peptide,” or “polypeptide” comprises a polymer of amino acid residues linked together by peptide bonds. The term refers to proteins, polypeptides, and peptides of any size, structure, or function. Typically, a protein will be at least three amino acids long. A protein may refer to an individual protein or a collection of proteins. Proteins preferably contain only natural amino acids, although non-natural amino acids (i.e., compounds that do not occur in nature but that can be incorporated into a polypeptide chain) and/or amino acid analogs as are known in the art may alternatively be employed. Also, one or more of the amino acids in a protein may be modified, for example, by the addition of a chemical entity such as a carbohydrate group, a hydroxyl group, a phosphate group, a farnesyl group, an isofarnesyl group, a fatty acid group, a linker for conjugation or functionalization, or other modification. A protein may also be a single molecule or may be a multi-molecular complex. A protein may be a fragment of a naturally occurring protein or peptide. A protein may be naturally occurring, recombinant, synthetic, or any combination of these.

The term “inhibit” or “inhibition” in the context of modulating level (e.g., expression and/or activity) of a target is not limited to only total inhibition. Thus, in some embodiments, partial inhibition or relative reduction is included within the scope of the term “inhibition.” In some embodiments, the term refers to a reduction of the level (e.g., expression, and/or activity) of a target to a level that is reproducibly and/or statistically significantly lower than an initial or other appropriate reference level, which may, for example, be a baseline level of a target. In some embodiments, the term refers to a reduction of the level (e.g., expression and/or activity) of a target to a level that is less than 75%, less than 50%, less than 40%, less than 30%, less than 25%, less than 20%, less than 10%, less than 9%, less than 8%, less than 7%, less than 6%, less than 5%, less than 4%, less than 3%. less than 2%, less than 1%, less than 0.5%, less than 0.1%, less than 0.01%, less than 0.001%. or less than 0.0001% of an initial level, which may, for example, be a baseline level of a target.

As used herein, the term “inhibitor” refers to an agent whose presence or level correlates with decreased level or activity of a target to be modulated. In some embodiments, an inhibitor may act directly (in which case it exerts its influence directly upon its target, for example by binding to the target); in some embodiments, an inhibitor may act indirectly (in which case it exerts its influence by interacting with and/or otherwise altering a regulator of a target, so that level and/or activity of the target is reduced). In some embodiments, an inhibitor is one whose presence or level correlates with a target level or activity that is reduced relative to a particular reference level or activity (e.g., that observed under appropriate reference conditions, such as presence of a known inhibitor, or absence of the inhibitor as disclosed herein, etc.).

The terms “composition” and “formulation” are used interchangeably.

A “subject” refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal. A “patient” refers to a human subject in need of treatment of a disease.

The term “biological sample” refers to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments or organelles (such as obtained by lysing cells and separating the components thereof by centrifugation or otherwise). Other examples of biological samples include blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample.

The terms “administer,” “administering.” or “administration” refers to implanting, absorbing, ingesting, injecting, inhaling, or otherwise introducing a compound described herein, or a composition thereof, in or on a subject.

The terms “treatment.” “treat.” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen). Treatment may also be continued after symptoms have resolved, for example, to delay and/or prevent recurrence.

The term “prevent.” “preventing,” or “prevention” refers to a prophylactic treatment of a subject who is not and was not with a disease but is at risk of developing the disease or who was with a disease, is not with the disease, but is at risk of regression of the disease. In certain embodiments, the subject is at a higher risk of developing the disease or at a higher risk of regression of the disease than an average healthy member of a population.

The terms “condition,” “disease,” and “disorder” are used interchangeably.

An “effective amount” of a compound described herein refers to an amount sufficient to elicit the desired biological response. An effective amount of a compound described herein may vary depending on such factors as the desired biological endpoint, the pharmacokinetics of the compound, the condition being treated, the mode of administration, and the age and health of the subject. In certain embodiments, an effective amount is a therapeutically effective amount. In certain embodiments, an effective amount is a prophylactic treatment. In certain embodiments, an effective amount is the amount of a compound described herein in a single dose. In certain embodiments, an effective amount is the combined amounts of a compound described herein in multiple doses.

A “therapeutically effective amount” of a compound described herein is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent.

A “prophylactically effective amount” of a compound described herein is an amount effective to prevent a condition, or one or more symptoms associated with the condition and/or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent.

A “proliferative disease” refers to a disease that occurs due to abnormal growth or extension by the multiplication of cells (Walker, Cambridge Dictionary of Biology; Cambridge University Press: Cambridge, UK, 1990). A proliferative disease may be associated with: 1) the pathological proliferation of normally quiescent cells; 2) the pathological migration of cells from their normal location (e.g., metastasis of neoplastic cells); 3) the pathological expression of proteolytic enzymes such as the matrix metalloproteinases (e.g., collagenases, gelatinases, and elastases); or 4) the pathological angiogenesis as in proliferative retinopathy and tumor metastasis. Exemplary proliferative diseases include cancers (i.e., “malignant neoplasms”), benign neoplasms, angiogenesis, inflammatory diseases, and autoimmune diseases.

The term “angiogenesis” refers to the physiological process through which new blood vessels form from pre-existing vessels. Angiogenesis is distinct from vasculogenesis, which is the de novo formation of endothelial cells from mesoderm cell precursors. The first vessels in a developing embryo form through vasculogenesis, after which angiogenesis is responsible for most blood vessel growth during normal or abnormal development. Angiogenesis is a vital process in growth and development, as well as in wound healing and in the formation of granulation tissue. However, angiogenesis is also a fundamental step in the transition of tumors from a benign state to a malignant one, leading to the use of angiogenesis inhibitors in the treatment of cancer. Angiogenesis may be chemically stimulated by angiogenic proteins, such as growth factors (e.g., VEGF). “Pathological angiogenesis” refers to abnormal (e.g., excessive or insufficient) angiogenesis that amounts to and/or is associated with a disease.

The terms “neoplasm” and “tumor” are used herein interchangeably and refer to an abnormal mass of tissue wherein the growth of the mass surpasses and is not coordinated with the growth of a normal tissue. A neoplasm or tumor may be “benign” or “malignant.” depending on the following characteristics: degree of cellular differentiation (including morphology and functionality), rate of growth, local invasion, and metastasis. A “benign neoplasm” is generally well differentiated, has characteristically slower growth than a malignant neoplasm, and remains localized to the site of origin. In addition, a benign neoplasm does not have the capacity to infiltrate, invade, or metastasize to distant sites. Exemplary benign neoplasms include, but are not limited to, lipoma, chondroma, adenomas, acrochordon, senile angiomas, seborrheic keratoses, lentigos, and sebaceous hyperplasias. In some cases, certain “benign” tumors may later give rise to malignant neoplasms, which may result from additional genetic changes in a subpopulation of the tumor's neoplastic cells, and these tumors are referred to as “pre-malignant neoplasms.” An exemplary pre-malignant neoplasm is a teratoma. In contrast, a “malignant neoplasm” is generally poorly differentiated (anaplasia) and has characteristically rapid growth accompanied by progressive infiltration, invasion, and destruction of the surrounding tissue. Furthermore, a malignant neoplasm generally has the capacity to metastasize to distant sites. The term “metastasis,” “metastatic.” or “metastasize” refers to the spread or migration of cancerous cells from a primary or original tumor to another organ or tissue and is typically identifiable by the presence of a “secondary tumor” or “secondary cell mass” of the tissue type of the primary or original tumor and not of that of the organ or tissue in which the secondary (metastatic) tumor is located. For example, a prostate cancer that has migrated to bone is said to be metastasized prostate cancer and includes cancerous prostate cancer cells growing in bone tissue.

The term “cancer” refers to a class of diseases characterized by the development of abnormal cells that proliferate uncontrollably and have the ability to infiltrate and destroy normal body tissues. See, e.g., Stedman's Medical Dictionary, 25th ed.; Hensyl ed.; Williams & Wilkins: Philadelphia, 1990. Exemplary cancers include, but are not limited to, hematological malignancies. The term “hematological malignancy” refers to tumors that affect blood, bone marrow, and/or lymph nodes. Exemplary hematological malignancies include, but are not limited to, leukemia, such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma, such as Hodgkin lymphoma (HL) (e.g., B-cell HL, T-cell HL) and non-Hodgkin lymphoma (NHL) (e.g., B-cell NHL, such as diffuse large cell lymphoma (DLCL) (e.g., diffuse large B-cell lymphoma (DLBCL, e.g., activated B-cell (ABC) DLBCL (ABC-DLBCL)))m follicular lymphoma, chronic lymphocytic leukemia/small lymphocytic lymphoma (CLL/SLL), mantle cell lymphoma (MCL), marginal zone B-cell lymphoma (e.g., mucosa-associated lymphoid tissue (MALT) lymphoma, nodal marginal zone B-cell lymphoma, splenic marginal zone B-cell lymphoma), primary mediastinal B-cell lymphoma, Burkitt lymphoma, Waldenström's macroglobulinemia (WM, lymphoplasmacytic lymphoma), hairy cell leukemia (HCL), immunoblastic large cell lymphoma, precursor B-lymphoblastic lymphoma, central nervous system (CNS) lymphoma (e.g., primary CNS lymphoma and secondary CNS lymphoma); and T-cell NHL, such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); lymphoma of an immune privileged site (e.g., cerebral lymphoma, ocular lymphoma, lymphoma of the placenta, lymphoma of the fetus, testicular lymphoma); a mixture of one or more leukemia/lymphoma as described above; myelodysplasia; and multiple myeloma (MM). Additional exemplary cancers include, but are not limited to, lung cancer (e.g., bronchogenic carcinoma, small cell lung cancer (SCLC), non-small cell lung cancer (NSCLC), adenocarcinoma of the lung); kidney cancer (e.g., nephroblastoma, a.k.a. Wilms' tumor, renal cell carcinoma); acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; ocular cancer (e.g., intraocular melanoma, retinoblastoma); familiar hypercosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); heavy chain disease (e.g., alpha chain disease, gamma chain disease, mu chain disease; hemangioblastoma; hypopharynx cancer; inflammatory myofibroblastic tumors; immunocytic amyloidosis; liver cancer (e.g., hepatocellular cancer (HCC), malignant hepatoma); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendoctrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

The term “immunotherapy” refers to a treatment of disease by inducing, enhancing, or suppressing an immune response. Immunotherapies designed to elicit or amplify an immune response are classified as activation immunotherapies, while immunotherapies that reduce or suppress an immune response are classified as suppression immunotherapies. Immunotherapy may encompass treatment with a molecular entity (e.g., immunotherapeutic agent) and/or a non-molecular entity (e.g., adoptive cell transfer).

The term “macrophage-directed immunotherapy” refers to an immunotherapy that derives its therapeutic effect by stimulating macrophages. Such stimulation can mobilize macrophage and myeloid components to destroy a tumor and its stroma, including the tumor vasculature. Macrophages can be induced to secrete antitumor cytokines and/or to perform phagocytosis, including antibody-dependent cellular phagocytosis.

The term “immunotherapeutic agent” refers to a molecular entity that induces, enhances, or suppresses an immune response. Immunotherapeutic agents include, but are not limited to, monoclonal antibodies, cytokines, chemokines, vaccines, small molecule inhibitors, and small molecule agonists.

The term “immune checkpoint inhibitor” refers to an agent that blocks certain proteins made by some types of immune system cells (e.g., T cells, macrophages) and some cancer cells. These proteins function to keep immune responses in check and can also function to keep immune system cells (e.g., T cells, macrophages) from killing cancer cells. When these proteins are blocked, immune system function is restored and the immune system is released enabling the desired immune system cells to kill cancer cells. Some immune checkpoint inhibitors are useful in treating cancer. A “macrophage immune checkpoint inhibitor” functions to stimulate macrophage phagocytosis of cancer cells. For example, CD47 is associated with a macrophage immune checkpoint (CD47/SIRPα as described herein). CD47-blocking therapies thus stimulate macrophage phagocytosis of cancer cells and are effective in treating cancer.

The terms “biologic,” “biologic drug,” and “biological product” refer to a wide range of products such as vaccines, blood and blood components, allergenics, somatic cells, gene therapy, tissues, nucleic acids, and proteins. Biologics may include sugars, proteins, or nucleic acids, or complex combinations of these substances, or may be living entities such as cells and tissues. Biologics may be isolated from a variety of natural sources (e.g., human, animal, microorganism) and/or may be produced by biotechnological methods and/or other technologies.

The term “antibody” refers to a functional component of serum and is often referred to either as a collection of molecules (antibodies or immunoglobulins) or as one molecule (the antibody molecule or immunoglobulin molecule). An antibody is capable of binding to or reacting with a specific antigenic determinant (the antigen or the antigenic epitope), which in turn may lead to induction of immunological effector mechanisms. An individual antibody is usually regarded as monospecific, and a composition of antibodies may be monoclonal (i.e., consisting of identical antibody molecules) or polyclonal (i.e., consisting of two or more different antibodies reacting with the same or different epitopes on the same antigen or even on distinct, different antigens). Each antibody has a unique structure that enables it to bind specifically to its corresponding antigen, and all natural antibodies have the same overall basic structure of two identical light chains and two identical heavy chains. Antibodies are also known collectively as immunoglobulins. An antibody may be of human or non-human (for example, rodent such as murine, dog, camel, etc) origin (e.g., may have a sequence originally developed in a human or non-human cell or organism), or may be or comprise a chimeric, humanized, reshaped, or reformatted antibody based, e.g., on a such a human or non-human antibody (or, in some embodiments, on an antigen-binding portion thereof).

In some embodiments, as will be clear from context, the term “antibody” as used herein encompasses formats that include epitope-binding sequences of an antibody, which such formats include, for example chimeric and/or single chain antibodies (e.g., a nanobody or Fcab), as well as binding fragments of antibodies, such as Fab, Fv fragments or single chain Fv (scFv) fragments, as well as multimeric forms such as dimeric IgA molecules or pentavalent IgM molecules. Also included are bispecific antibodies, bispecific T cell engagers (BiTEs), immune mobilixing monoclonal T cell receptors against cancer (ImmTACs), dual-affinity re-targeting (DART); alternative scaffolds or antibody mimetics (e.g., anticalins, FN3 monobodies, DARPins, Affibodies, Affilins, Affimers, Affitins, Alphabodies, Avimers, Fynomers, Im7, VLR, VNAR, Trimab, CrossMab, Trident); nanobodies, binanobodies, F(ab′)2, Fab′, di-sdFv, single domain antibodies, trifunctional antibodies, diabodies, and minibodies.

The term “therapeutic agent” refers to an agent having one or more therapeutic properties that produce a desired, usually beneficial, effect. For example, a therapeutic agent may treat, ameliorate, and/or prevent disease. In some embodiments, a therapeutic agent may be or comprise a biologic, a small molecule, or a combination thereof.

The term “chemotherapeutic agent” refers to a therapeutic agent known to be of use in chemotherapy for cancer.

The term “targeted agent” refers to an anticancer agent that blocks the growth and spread of cancer by interfering with specific proteins (“molecular targets”) that are involved in the growth, progression, and spread of cancer. Targeted agents are sometimes called “targeted therapies,” “targeted cancer therapies,” “molecularly targeted drugs,” “molecularly targeted therapies,” or “precision medicines.” Targeted agents differ from standard chemotherapy in that targeted agents act on specific molecular targets that are associated with cancer, whereas many chemotherapeutic agents act on all rapidly dividing cells (e.g., whether or not the cells are cancerous). Targeted agents are deliberately chosen or designed to interact with their target, whereas many standard chemotherapies are identified because they may indiscriminantly kill cells.

A “cell” or “target cell” as used herein, may be present in a population of cells (e.g., in a tissue, an organ, or an organoid). In some embodiments, a population of cells is composed of a plurality of cell types. Cells for use in the methods of the present disclosure can be present within an organism, a single cell type derived from an organism, or a mixture of cell types. Included are naturally occurring cells and cell populations, genetically engineered cell lines, cells derived from transgenic animals, etc. Virtually any cell type and size can be accommodated in the methods and systems described herein. Suitable cells include bacterial, fungal, plant, and animal cells. In some embodiments, the cells are mammalian cells (e.g., complex cell populations such as naturally occurring tissues). In some embodiments, the cells are from a human. In certain embodiments, the cells are collected from a subject (e.g., a human) through a medical procedure such as a biopsy. Alternatively, the cells may be a cultured population (e.g., a culture derived from a complex population or a culture derived from a single cell type where the cells have differentiated into multiple lineages).

In particular, the term “target cell” refers to a cell (e.g., inflicted cells such as cancer cells, infected cells, etc.) that is targeted for phagocytosis by a macrophage. The target cell can be any cell as described herein. Examples of target cells include but are not limited neoplastic cells, such as cancer cells, and disease associated cells. Examples of cancers include but are not limited to solid tumors, breast cancer, pancreatic cancer, colon cancer, non-small cell lung cancer, leukemias, lymphomas, such as mantle cell lymphoma, and myelomas, such as multiple myeloma. The term “cancer cells” can also be used to refer to cancer or tumor cell lines.

As used herein, the term “gene” refers to a nucleic acid fragment that expresses a protein, including regulatory sequences preceding (5′ non-coding sequences) and following (3′ non-coding sequences) the coding sequence. “Native gene” refers to a gene as found in nature with its own regulatory sequences.

The terms “assessing”, “determining”, “evaluating”, and “assaying” are used interchangeably herein to refer to any form of detection or measurement, and include determining whether a substance, signal, enzymatic activity, disease, condition, etc., is present or not. The result of an assessment may be expressed in qualitative and/or quantitative terms. Assessing may be relative or absolute. “Assessing the presence of” includes determining the amount of something that is present or determining whether it is present or absent.

### BRIEF DESCRIPTION OF THE DRAWINGS

FIG. 1: Establishing a high-throughput assay to evaluate macrophage-mediated cytotoxicity. Left, GFP+ target cells (in this case, 2.500 human PC9 cells) were combined with primary human macrophages (10,000 cells per well). Cells were combined in 384-well plates. Wells were treated with vehicle control or an anti-CD47 antibody as an example of a therapy that activates macrophages to attack cancer cells. Whole-well imaging of phase contrast and GFP fluorescence channel were acquired every 4 hours over time using a Sartorius Incucyte S3 Live Cell Imaging System. Automated image analysis was used to create an image mask of the GFP+ channel to quantify GFP+ area over time. Right, Growth curves of GFP+ PC9 cells over time in response to vehicle control or an anti-CD47 antibody (macrophage immune checkpoint inhibitor) as quantified by GFP+ area over time. The greatest separation in growth curves occured after 24 hours of co-culture.

FIG. 2: Representative example of a high-throughput small molecule drug screen to identify activators or inhibitors of macrophage-mediated cytotoxicity. Primary human macrophages are combined with GFP+ target cells (in this case, 2,500 GFP+ PC9 cancer cells and 10,000 primary human macrophages) in 384-well plates. An anti-CD47 antibody is then added to every well at a final concentration of 10 ug/mL to activate macrophage-mediated cytotoxicity of the target cells. A different small molecule drug compound is then added to each well (in this example, a library of 800 FDA approved drugs). The cells are then co-cultured for a period of up to 7 days, with greatest differences in growth curves observed with at least 24 hours of co-culture. During the co-cultured period, whole-well imaging of phase contrast and GFP+ fluorescence channel is performed. GFP+ area (representing target cell viability and survival when co-cultured with macrophages) is quantified over time by automated image analysis.

FIG. 3: Representative example of one 384 well plate from drug screen. Image shows GFP+ channel for every well of one 384 well plate. Each well contains an individual co-culture of GFP+ target cells (PC9 cells), primary human macrophages, and an anti-CD47 antibody (10 ug/mL). Every well from column 3-22 contains a different FDA approved drug compound used at 5 uM concentration. Columns 1-2 and 23-24 are control wells. Macrophage Inhibitor compounds were identified as those that promote growth of GFP+ target cells over time (for example, wells G8, K8). Macrophage activator compounds were identified as compounds that inhibit growth of GFP+ target cells over time (for example, wells E6, G4).

FIG. 4: Representative example of one 384 well plate from drug screen. Image shows measured image mask of GFP+ channel for every well of one 384 well plate. Each well contains an individual co-culture of GFP+ target cells (PC9 cells), primary human macrophages, and an anti-CD47 antibody (10 ug/mL). Every well from columns 3-22 contains a different FDA approved drug compound used at 5 uM concentration. Columns 1-2 and 23-24 are control wells. Macrophage Inhibitor compounds were identified as those that promote growth of GFP+ target cells over time (for example, wells G8, K8). Macrophage activator compounds were identified as compounds that inhibit growth of GFP+ target cells over time (for example, wells E6, G4).

FIGS. 5A-5B: Representative examples of results of drug screens. FIG. 5A is a plot showing representative example of results of a drug screen using 800 FDA-approved drugs co-cultured with macrophages, GFP+ PC9 cancer cells, and an anti-CD47 antibody. Drugs were ranked based on their ability to kill cancer cells in the presence of macrophages with GFP+ PC9 target cells versus PC9 cells alone (Target cell survival index). Alprostadil was identified as a representative macrophage inhibitor. Celecoxib was identified as a representative macrophage activator. FIG. 5B shows a series of charts showing results of a separate run of the drug screen in which drugs were quantified as hits based on their ability to inhibit macrophage function. Bottom chart shows the categories of hits from the screen identifying macrophage inhibitor drugs.

FIG. 6: Validation studies demonstrating the high-throughout screening assay can identify drugs that alter macrophage function. Left plot shows a validation study using high-throughput co-culture assays with primary human macrophages and GFP+ DLD-1 colon cancer cells as targets. Cells were co-cultured with an anti-CD47 antibody (10 ug/mL) to activate the macrophages to attack the target cells and varying concentrations of alprostadil, a macrophage inhibitor identified from screening efforts. GFP+ area (representing target cell viability) was scored as a measure of macrophage-mediated cytotoxicity on day 6.5 of co-culture. A dose-response relationship exists between alprostadil and its ability to inhibit macrophage-mediated cytotoxicity. Right plot shows a validation study using high-throughput co-culture assays with primary human macrophages and GFP+ PC9 lung cancer cells as targets. Cells were co-cultured with an anti-CD47 antibody (10 ug/mL) to activate the macrophages to attack the target cells and varying concentrations of celecoxib, a macrophage activator identified from our screening efforts. GFP+ area (representing target cell viability) was scored as a measure of macrophage-mediated cytotoxicity on day 6.5 of co-culture. A dose-response relationship exists between celecoxib and its ability to activate macrophage-mediated cytotoxicity.

FIG. 7: Summary results of drug screen to identify enhancers of macrophage-mediated cytotoxicity. An 800 FDA drug compound library was screened as individual experiments 5 times, each time using a different primary macrophage donor. GFP+ PC9 cells were used as target cells in each run. Left plot is a volcano plot depicting phenotypic effect size for each drug of 800 FDA-approved drug compound library (x-axis) versus statistical significance scores (y-axis) across all runs of the library. Each point is a different drug from the library. Phenotypic effect size was normalized to the ability of drugs to kill cancer cells in the presence of macrophages and anti-CD47 compared to the ability to kill cancer cells alone (“macrophage/control”). Hashed lines represent 4-fold change in effect size (x-axis) or p<0.05 by t test (y-axis). EGFR TKIs (gefitinib, erlotinib) were identified as the top enhancers of macrophage-dependent destruction of PC9 cells. Right plot shows growth curves of GFP+target cell area over time depicted as a ratio of growth in the presence of macrophages plus anti-CD47 versus cancer cells alone (“Mphi/control”). Each curve represents a different drug compound. Gefitinib and Erlotinib were identified as drugs that enhance macrophage-mediated cytotoxicity of target cells. The greatest effect size is identified after ˜24 hours of co-culture.

FIG. 8: Growth of GFP+ target cells from FDA-compound library screen. Growth is measured by GFP+ area and normalized to GFP+ area at time t=0 hours without normalization cancer only control wells. Two drugs that enhanced macrophage-dependent killing of the target cells are indicated (gefitinib, erlotinib). Black lines with grey shading indicates 95% confidence interval of 384 wells from drug library.

FIGS. 9A-9B: High-throughput macrophage-cytotoxicity can be used to screen libraries of recombinant proteins. A high-throughput screen was performed using a library of 114 recombinant human cytokines. The screen was independently performed with GFP+ PC9 cells or GFP+ DLD-1 cells as targets. Primary human macrophages were used as effector cells. For each cell line, co-cultures were performed with vehicle control or an anti-CD47 antibody (10 ug/mL). AUC of GFP+ cancer cell growth was evaluated over time as a measure of cancer cell growth. Each plot indicates results with PC9 cells on x-axis and results with DLD-1 cells on y-axis. Each point represents a replicate of an individual cytokine from the library (each tested as three technical replicates). FIG. 9A shows results of baseline interactions between macrophages and cancer cells as influenced by each cytokine of the library. FIG. 9B shows results of co-culture assays treated with an anti-CD47 antibody.

FIG. 10: High-throughput macrophage-mediated cytotoxicity assay is used to evaluate macrophage immune-checkpoint inhibitor drugs. The diagram on the left shows known macrophage immune checkpoint molecules that regulate macrophage-mediated cytotoxicity of cancer cells. The plot on the right shows a high-throughput assay was performed to rank the effects of different macrophage immune checkpoint inhibitors as single agents or in combination with an EGFR tyrosine kinase inhibitor (osimertinib). Primary human macrophages were co-cultured with GFP+ PC9 cells (an EGFR mutant lung cancer cell line) in this experiment. Each dot represents a co-culture well from 4-8 macrophage donors performed using 3-4 technical replicates per condition. GFP+ area was measured on day 6.5 of co-culture. All macrophage immune checkpoint antibody therapies were used at 10 ug/mL. Osimertinib was used at 100 nM.

FIG. 11: Example of In Vitro Validation Assays Using Primary Human Macrophages versus Primary Tumor Associated Macrophages. High-throughput macrophage-mediated cytotoxicity assay can be performed with primary tumor-associated macrophage. High-throughput macrophage cytotoxicity assay performed with mCherry+ PC9 cancer cells as targets. Cancer cells stably express a Cas9 inactivation construct to permit CRISPR inactivation. Prior to co-culture with macrophages, cancer cells were transduced with lentivirus encoding control. CD47, or Ermp1 sgRNAs for CRISPRi knockdown (KD) and selected for sgRNA expression for approximately 5 days using puromycin. The plot on the left shows a co-culture assay using primary human macrophages from healthy human blood donors. Wells were treated with vehicle control (PBS) or an anti-CD47 antibody (10 ug/mL) to activate macrophage-mediated cytotoxicity. The plot on the right shows assays performed with primary human tumor-associated macrophages isolated from a malignant pleural effusion specimen from a patient with lung cancer. Tumor associated macrophages show similar results and functional capacity as primary human macrophages from healthy blood donors.

FIG. 12: Engineering stem cell-derived macrophages for cancer immunotherapy. High-throughput macrophage-mediated cytotoxicity assay can be used to evaluate stem-cell derived macrophages. Macrophages were differentiated from established embryonic stem cell lines. Two different cell lines were tested as target cells (left plot shows GFP+ DLD-1 colon cancer cells; right plot shows GFP+ lung cancer cells). For each experiment, varying numbers of macrophages per well were tested (5,000, 10,000, or 20,000 macrophages per well) with a constant number of target cells (7.500 DLD-1 cells or 2.500 PC9 cells). For each experiment, cells were co-cultured with vehicle control (PBS) or an anti-CD47 antibody (10 ug/mL). Useful results were observed with >5,000 macrophages per well. GFP+ area was measured over time as a representation of target cell growth and survival. The greatest separation of growth curves was observed after 24 hours of co-culture.

FIG. 13: Competition assays using two different cell lines that vary in their sensitivity to macrophage-mediated cytotoxicity. A co-culture assay was performed using RFP+ CD47 knockout PC9 cells (which are resistant to anti-CD47) and wild-type GFP+ PC9 cells in combination with 10,000 primary human macrophages. Whole well imaging was performed over time and analyzed by automated image analysis for both RFP+ fluorescent area and GFP+ fluorescent area. On the left are representative images of wells after 6.5 days of co-culture. Rows show different treatments conditions: vehicle control (PBS), anti-CD47 antibody (10 ug/mL), or the anti-EGFR antibody cetuximab (10 ug/mL). Columns show independent replicates (Rep). The plot on the right shows quantification of fluorescent area for each RFP and GFP in response to each therapeutic condition. Numbers above bars indicate ratio of RFP+ area to GFP+ area. Anti-CD47 preferentially kills the GFP+ cells and has minimal effect on the RFP+CD47 knockout cells. Cetuximab stimulates macrophage cytotoxicity of both GFP+ and RFP+ lines, with slight preference for RFP+ CD47 knockout cells versus GFP+ cells. These studies could be use or further adapted to screen drugs that differentially activate or inhibit macrophage killing between to different cell types directly compared in competition in vitro.

FIG. 14: High-throughput macrophage-mediated cytotoxicity assay can be used to validate genes as novel macrophage immune checkpoints. The plot on the left shows results of an unbiased genetic screen which identified genes that correlate with sensitivity or resistance to macrophage-mediated cytotoxicity. The plot on the right shows validation of genetic targets performed using the 384-well high-throughput assay to validate genetic targets identified from correlation. Primary human macrophages were combined with GFP+ PC9 cells as targets. Prior to co-culture, PC9 cells had been transfected with recombinant Cas9 and ribonucleoprotein complexes containing sgRNAs to genes identified from the unbiased screening effort. PC9 cells were then co-cultured for approximately 5 days before being exposed to primary human macrophages in the presence of an anti-CD47 antibody (10 ug/ml). Each curve represents an individual gene that was deleted via CRISPR/Cas9 knockout. All genes were tested in a single assay to compare them directly and rank their ability to alter macrophage function. Black is control sgRNA (curve marked with “x”). The top curve marked with “-” was defined as a novel “cat me” signal that made target cells more resistant to macrophage-mediated cytotoxicity when deleted. The remaining bottom curves were defined as novel “don't eat me” signals that made target cells more vulnerable to macrophage-mediated cytotoxicity when deleted (bottom curves at 96 hours). The greatest separation of experimental sgRNA curves from control sgRNA curve occurred >24 hours of co-culture.

FIGS. 15A-15M: An unbiased compound library screen identifies synergy between targeted therapy and macrophage-directed immunotherapy for EGFR mutant lung cancer. FIG. 15A depicts the experimental setup of an unbiased functional screen to identify drugs that synergize macrophage-directed immunotherapy. Primary human macrophages were co-cultured in 384-well plates with GFP+ PC9 cancer cells (a human EGFR mutant NSCLC cell line). The specimens were treated with an anti-CD47 antibody, and then a drug compound library was added (n=800 FDA-approved drugs). The cells were incubated for 3-5 days and GFP+ area was quantified by automated microscopy and image analysis. As controls, GFP+ PC9 cells were cultured with each individual drug of the library alone for comparison. FIG. 15B depicts histograms showing CD47 expression on the surface of NSCLC cell lines containing the indicated driver mutations as assessed by flow cytometry. FIG. 15C depicts flow cytometric analysis of macrophage immune checkpoint molecules on the surface of NSCLC cell lines and patient-derived specimens containing the indicated driver mutations. Geometric mean fluorescence intensity (Geo. MFI) for each antigen was compared to CD47 by one-way ANOVA with Holm-Sidak multiple comparison test, ns, not significant. **p<0.01. FIG. 15D depicts bar graphs showing CD47 expression on EpCam+ cancer cells from malignant pleural effusions specimens of patients with NSCLC containing the indicated driver mutations. Left, percent of CD47-positive cells. Right, CD47 geometric MFI. The data represent the mean±SD from 3 technical replicates of n=10 independent patient specimens (patients 1, 4, 6, 7, and 9: EGFR; patients 2, 3, and 8: KRAS; patients 5and 10: ALK). FIG. 15E shows phagocytosis assays using primary human macrophages and CFSE-labelled PC9 cells. The PC9 cells were exposed to vehicle control (PBS) or 1 uM EGFR TKI (erlotinib, gefitinib, or osimertinib) for 24 hours. The cells were then collected and co-cultured with primary human macrophages±an anti-CD47 antibody for 2 hours. Phagocytosis was measured by flow cytometry as the percentage of macrophages containing engulfed CFSE+ PC9 cells. Data were normalized to the maximal response for each independent donor. Data depict mean±SD from n=4 independent blood donors, ns, ***p<0.001, ****p<0.0001 by one-way ANOVA with Holm-Sidak multiple comparison test. FIG. 15F depicts bar graphs showing phagocytosis assays performed using GFP+ PC9 cells exposed to 1 uM osimertinib for varying amounts of time prior to co-culture with primary human macrophages. The cells were collected and analyzed for phagocytosis as in F. **P<0.01, ***p<0.001, ****p<0.0001 by two-way ANOVA with Holm-Sidak multiple comparison test. FIG. 15G is a graph showing phagocytosis assays performed using GFP+ PC9 cells exposed to varying concentrations of osimertinib for 24 hours prior to co-culture with human macrophages. The data represent the mean±SD from 3 technical replicates representing n=8 independent macrophage donors combined from n=2 independent experiments. FIG. 15H presents representative images of whole-well microscopy showing GFP+ area as quantified by automated image analysis from wells treated with drugs found to enhance (erlotinib, gefitinib) or inhibit (dexamethasone) macrophage-dependent cytotoxicity of PC9 cells. The scale bar represents 800 um. FIG. 15I shows a volcano plot summarizing the results of the drug library screen. Each point represents the mean of each individual drug treatment condition from n=5 experimental trials. The phenotypic effect size (x-axis) is depicted as logz fold-change of GFP+ area in the macrophage+ anti-CD47 condition relative to PC9 cells alone. Values were normalized to account for variation due to well position. The dashed lines represent 2-fold change in effect size (x-axis) or p<0.05 by t test (y-axis). Gefitinib and erlotinib were identified as the top enhancers of macrophage-dependent cytotoxicity of PC9 cells, whereas bortezomib, idarubicin, famciclovir, dasatanib, methylprednisolone, vincristine sulfate, mitoxantrone, amcinonide, and auranofin inhibited macrophage dependent cytotoxicity or were drugs that macrophages protected against. FIG. 15J presents the representative curves showing macrophage-dependent cytotoxicity over time as represented by decreases in GFP+ area of macrophage+ anti-CD47 condition relative to the control condition. Curves from one representative plate showing gefitinib and erlotinib enhance macrophage-dependent cytotoxicity within ˜48 hours. The dashed lines indicate the empirical 95% tolerance interval. FIG. 15K is a box and whisker plot of drug classes included in the screen as ranked by normalized log₂ fold-change of GFP+ area in macrophage versus PC9 control condition. Each box indicates the median, interquartile range, maxima and minima (excluding outliers) for the indicated drug class. Drug classes that significantly increased relative GFP+ area are anthracyclines, steroids, retinoids, and chemotherapies, whereas EGFR TKIs were identified as the only drug class that significantly decreased relative GFP+ area. Each class of drugs was compared with controls (DMSO and empty wells) using a t test (**FDR<0.01, ***FDR<0.001). FIG. 15L shows representative examples of phagocytosis assays using primary human macrophages and GFP+ PC9 cells. The PC9 cells were exposed to vehicle control (PBS) or 1 uM EGFR TKI (erlotinib, gefitinib, or osimertinib) for 24 hours. The cells were then collected and co-cultured with primary human macrophages±an anti-CD47 antibody for 2 hours. Phagocytosis was measured by flow cytometry as the percentage of macrophages containing engulfed GFP+ PC9 cells as indicated in plots. FIG. 15M shows the quantification of phagocytosis assays using the indicated EGFR TKIs at 1 uM concentration. Phagocytosis was normalized to the maximal response for each independent donor. Data depict mean±SD from n=9 independent blood donors combined from 3 independent experiments using CFSE+ or GFP+ PC9 cells.

FIGS. 16A-16F: Combining TKIs with anti-CD47 antibodies eliminates EGFR mutant persister cells in long-term co-cultures assays with human macrophages. FIG. 16A shows representative images of GFP+ channel from co-culture assays on day 6.5. GFP+ PC9 cells were co-cultured in 384-well plates with primary human macrophages in the presence or absence of anti-CD47 antibodies (10 ug/mL) and the indicated EGFR TKIs (1 uM). Whole-well imaging and automated image analysis was performed to quantify GFP+ area per well over time. Scale bar, 800 um. FIG. 16B is a representative plot showing growth curves of GFP+ PC9 cells in co-culture with primary human macrophages over time. The cells were treated with 1 uM osimertinib and/or 10 ug/mL anti-CD47 antibody as indicated. The data represent the mean±SEM of three technical replicates per donor using n=9 independent macrophage donors. Data are combined from two independent experiments. ***p<0.001, ****p<0.0001 by two-way ANOVA with Holm-Sidak multiple comparison test on day 14 of co-culture. FIG. 16C shows growth of GFP+ PC9 cells in co-culture with primary human macrophages in the presence or absence of anti-CD47 antibodies (10 ug/mL) and the indicated EGFR TKIs (1 uM). Points represent individual replicates, bars represent mean. Data represent three technical replicates per donor using n=9 independent macrophage donors combined from two independent experiments. ***p<0.001. ****p<0.0001 by one way ANOVA with Holm-Sidak multiple comparison test. FIG. 16D shows growth of GFP+ MGH119 patient-derived cells in co-culture with primary human macrophages in the presence or absence of anti-CD47 antibodies (10 ug/mL) and the indicated EGFR TKIs (1 uM). Points represent individual replicates, bars represent mean. Data represent three technical replicates per donor using n=3 independent macrophage donors, ns, not significant, *p<0.05, **p<0.01, ***p<0.001, ****p<0.0001 by one-way ANOVA with Holm-Sidak multiple comparison test. FIG. 16E shows growth of GFP+ MGH134 patient-derived cells in co-culture with primary human macrophages in the presence or absence of anti-CD47 antibodies (10 ug/mL) and the indicated EGFR TKIs (1 uM). MGH134 cells are resistant to first-generation EGFR TKIs (erlotinib, gefitinib) but sensitive to third generation TKIs (osimertinib). Points represent individual replicates, bars represent mean. Data represent three technical replicates per donor using n=4 independent macrophage donors, ns, not significant, *p<0.05, **p<0.01, ***p<0.001, ****p<0.0001 by one-way ANOVA with Holm-Sidak multiple comparisons test. FIG. 16F shows growth of GFP+ PC9 cells in co-culture assays with primary human macrophages and the indicated macrophage immune checkpoint inhibitors (10 ug/mL). Cells were cultured with antibodies alone or in combination with 100 nM osimertinib. Points represent individual replicates, bars represent mean. Data represent GFP+ area on day 6.5 with 3-4 technical replicates per donor and n=4-8 independent macrophage donors, ns, not significant, *p<0.05, **p<0.01, ***p<0. 001, ****p<0.0001 by two-way ANOVA with Holm-Sidak's multiple comparisons test.

FIGS. 17A-17G: Targeted inhibition of the MAPK pathway primes NSCLC cells for macrophage-mediated destruction. FIG. 17A shows growth of GFP+ NCI-H3122 (a human ALK rearranged NSCLC cell line) in co-culture with primary human macrophages in the presence or absence of anti-CD47 antibodies (10 ug/mL) and the indicated ALK-specific TKIs (1 uM). Points represent individual replicates, bars represent mean. Data represent three technical replicates per donor using n=4 independent macrophage donors. *p<0.05, **p<0.01, ***p<0.001, ****p<0.0001 by one-way ANOVA with Holm-Sidak multiple comparisons test. FIG. 17B shows growth of GFP+ NCI-H3122 cells in co-culture with primary human macrophages in the presence or absence of anti-CD47 antibodies (10 ug/mL) and varying concentrations of the ALK-specific TKI lorlatinib. Data represent mean±SD of three replicates each from n=4 independent macrophage donors on day 6.5 of co-culture. IC₅₀ of lorlatinib alone (PBS)=10.29 nM (95% CI [8.665. 12.22]) versus IC₅₀ of lorlatinib+anti-CD47=2.135 nM (95% CI [0.6934, 6.261]). FIG. 17C shows growth of GFP+ NCI-H358 (a human KRAS G12C mutant NSCLC cell line) in co-culture with primary human macrophages in the presence or absence of anti-CD47 antibodies (10 ug/mL) and the indicated KRAS G12C-specific inhibitors (1 uM). Data represent mean±SEM from three technical replicates per donor using n=4 independent macrophage donors, *p<0.05, **p<0.01, ***p<0.001, ****p<0.0001 by one-way ANOVA with Holm-Sidak multiple comparisons test. FIG. 17D shows growth of GFP+ NCI-H358 cells in co-culture with primary human macrophages in the presence or absence of anti-CD47 antibodies (10 ug/mL) and varying concentrations of the KRAS G12C-specific inhibitor sotorasib. Data represent mean±SD of three replicates each from n=4 independent macrophage donors on day 6.5 of co-culture. IC₅₀ of sotorasib alone (PBS)=896.5 nM (95% CI [558.6, 1697]) versus IC₅₀ of sotorasib+anti-CD47=10.30 nM (95% CI [2.949. 40.48]). FIG. 17E is a diagram depicting the EGFR-RAS-MAPK signaling pathway. KRAS activation leads to bifurcation of signaling via downstream MAPK pathway elements or the PI3K-AKT pathway. Drugs (rounded boxes) indicate specific inhibitors of pathway clements used in this study. FIG. 17F shows growth of GFP+ PC9 cells in co-culture with primary human macrophages in the presence or absence of anti-CD47 antibodies (10 ug/mL) and varying EGFR-RAS-MAPK or PI3K-AKT pathway inhibitors as indicated in FIG. 17E. Points represent individual replicates, bars represent mean. Data represent three technical replicates per donor using n=4 independent macrophage donors on day 6.5 of co-culture.,ns, not significant, *p<0.05, ****p<0.0001 by one-way ANOVA with Holm-Sidak multiple comparisons test. FIG. 17G shows growth of GFP+ NCI-H358 cells in co-culture with primary human macrophages in the presence or absence of anti-CD47 antibodies (10 ug/mL) and varying EGFR-RAS-MAPK or PI3K-AKT pathway inhibitors as indicated in FIG. 17E. Points represent individual replicates, bars represent mean. Data represent three technical replicates per donor using n=4 independent macrophage donors on day 6.5 of co-culture, ns, not significant, *p<0.05, **P<0.01, ***p<0.0001 by one-way ANOVA with Holm-Sidak multiple comparisons test.

FIGS. 18A-18F: The combination of targeted therapy and CD47 blockade enhances anti-tumor responses in mouse tumor models. FIG. 18A shows an EGFR mutant xenograft model of PC9 cells engrafted into NSG mice. Tumors were allowed to grow to 500 mm³ and then mice were randomized to treatment with vehicle control, anti-CD47 antibodies (250 ug three times weekly), osimertinib (5 mg/kg five times weekly), or the combination of anti-CD47 plus osimertinib. Tumor volumes were measured over time. Data depict mean tumor volume±SEM (left), growth curves of individual mice (middle), or percent change in tumor volume from baseline (right). Complete responses were observed in 4/10 mice (40%) in the combination cohort. Data represent n=9-11 mice per cohort combined from two independent experiments. FIG. 18B shows an EGFR mutant NSCLC xenograft model of MGH134-1 patient-derived cells engrafted into NSG mice and treated as in FIG. 18A. Data represent growth curves of percent change in tumor volume from baseline with mean±SEM of n=4 mice per cohort from one experiment. FIG. 18C shows an ALK-positive xenograft model of NCI-H3122 cells engrafted into NSG mice and treated with vehicle control, anti-CD47 antibodies (250 ug three times weekly), lorlatinib (6 mg/kg five times weekly), or the combination of anti-CD47 antibodies and lorlatinib. Data represent percent change in tumor volume from baseline with mean±SEM of n=4 mice per cohort from one experiment. FIG. 18D shows a KRAS G12C mutant xenograft model of NCI-H358 cells engrafted into NSG mice and treated with vehicle control, anti-CD47 antibodies (250 ug three times weekly), sotorasib (100 mg/kg five times weekly), or the combination of anti-CD47 antibodies and sotorasib. Data represent percent change in tumor volume from baseline with mean±SEM of n=4 mice per cohort from one experiment. FIG. 18E shows a syngeneic tumor model of KRAS G12C mutant lung cancer using wild-type 3LL ΔNRAS cells or a CD47-knockout variant engrafted into C57BL/6 mice. The mice were treated with vehicle control or sotorasib (30 mg/kg five times weekly). Points indicate individual tumor volumes, bars represent median with n=9-10 mice per treatment cohort from one experiment, ns, not significant, *p<0.05 by paired t test for the indicated comparisons. FIG. 18F shows a syngeneic tumor model of KRAS^{G12C} mutant lung cancer using wild-type 3LL ΔNRAS cells or a CD47-knockout variant engrafted into C57BL/6 mice. The mice were treated with vehicle control or sotorasib (30 mg/kg five times weekly) starting on day 7 post-engraftment. The points indicate individual tumor volumes and the bars represent median with n=9-10 mice per treatment cohort, ns, not significant, *p<0.05 by paired t test for the indicated comparisons. FIGS. 18A-18D: **p<0.01 by unpaired t test for combo versus targeted therapy.

FIGS. 19A-19I: Targeted therapies induce cross-sensitization to anti-CD47therapy and downregulate B2M and CD73. FIG. 19A is a diagram showing generation of GFP+ cell lines that are resistant to targeted therapies. For each parental cell line (PC9, NCI-H3122, or NCI-H358), cells were cultured in the presence of 1.0 uM of appropriate targeted therapy for prolonged duration until resistant cells emerged and proliferated in culture. FIGS. 19B-19D show long-term co-culture assays using GFP+ PC9 cells (FIG. 19B), GFP+ NCI-H3122 cells (FIG. 19C), or GFP+ NCI-H358 cells (FIG. 19D) that are resistant to the indicated targeted therapies. In each case, anti-CD47 therapy resulted in significant enhancement in macrophage-mediated cytotoxicity relative to the naïve parental lines. Experiments performed once with 4 independent donors (erlotinib, gefitinib, osimertinib. alectinib, crizotinib resistant lines) or twice with a total of 8 independent donors (lorlatinib. sotorasib resistant lines) with 3 technical replicates per donor. The bars represent means from analysis at 6.5 days of co-culture. FIG. 19E shows a scatter plot with the results of comprehensive surface immunophenotyping of parental NCI-H358 cells versus a GFP+ sotorasib-resistant variant. Each dot represents the normalized mean fluorescence intensity (nMFI) of an individual surface antigen from a total of 354 specificities tested in one experiment. Antigens that exceed the 95% predicted interval for expression on the parental line or resistant line are indicated. FIG. 19F shows how the treatment of parental NCI-H358 cells with the indicated targeted therapies causes downregulation of B2M (top) and CD73 (bottom) over time as measured by flow cytometry. ****p<0.0001 for each drug treatment condition compared to time=0 h by one-way ANOVA with Tukey's multiple comparison test. FIG. 19G shows the evaluation of wild-type versus B2M KO lung cancer cell lines in long-term co-culture assays with human macrophages. FIG. 19H shows the evaluation of wild-type versus CD73 KO PC9 cells in long-term co-culture assays with human macrophages. FIG. 19I shows the treatment of PC9 cells with a CD73-blocking antibody alone or in combination with anti-CD47 in long-term co-culture assays with human macrophages. For FIGS. 19G-19I, the data represent at least two independent experiments performed with 6-12 independent macrophage donors. For FIGS. 19B-19D and 19G-19I, ns, not significant, *p<0.05, **p<0.001, ***p<0.001, ****p<0.0001 by two-way ANOVA with Holm-Sidak multiple comparisons test: #GFP+ area underrepresented due to high confluency of wells and was not visually different by phase microscopy.

FIGS. 20A-20B: The composition of an FDA-approved drug library used for screening efforts. FIG. 20A is a chart depicting the drug classes that were included in the screening library. Percentages indicate number of drugs per class from a total of N=800 individual drug wells and including 14 DMSO controls. FIG. 20B shows a table depicting the number and percentage of drugs from each class that was included in the screening library.

FIGS. 21A-21D: Representative images of wells from small molecule screen using FDA-approved drug library. GFP+ PC9 cells were combined with primary human macrophages and the indicated drug therapies in 384-well plates. Representative images are shown from a single experimental run of the full FDA-approved drug library using macrophages derived from an individual blood donor at t=3 d 16 h. FIG. 21A shows the whole well imaging of the GFP+ channel from wells treated with the indicated therapies. Erlotinib and gefitinib were identified as drugs that enhance macrophage-dependent cytotoxicity of PC9 cells, while dexamethasone and other steroid compounds were identified as inhibitors of macrophage-dependent cytotoxicity. FIG. 21B shows an image mask of GFP+ pixels used for quantification and analysis. FIG. 21C shows the overlay of GFP+ channel with phase contrast imaging. FIG. 21D presents phase contrast imaging which shows the confluency of wells with GFP+ PC9 cells and primary human macrophages present.

FIGS. 22A-22E: Analysis of high-throughput screen reveals differential activity of drugs from the FDA-approved library. FIG. 22A is a scatter plot showing how drugs affect growth of GFP+ PC9 cells alone (x-axis) versus when they are co-cultured with macrophages and anti-CD47 therapy (y-axis). The points are distinguished by density from low to high, and the majority of the drugs are localized near the origin, which indicates no activity affected either condition. The diagonal identity line indicates where drugs affect PC9 cells equally under both treatment conditions. The majority of drugs have no significant effect under either condition. The circled data represents the 95% of drugs with outliers. The indicated 95% tolerance interval (TI) was constructed after fitting the joint density to a single two-dimensional Gaussian distribution. Drugs indicated in the graph were identified as hits based on statistical significance and >2-fold change in effect size between the two treatment conditions (see FIG. 15J). Erlotinib and gefitinib were significantly enhanced macrophage-dependent killing of GFP+ PC9 cells. All of the other drugs indicated in the graph resulted in more cancer cell growth of PC9 cells in the presence of macrophages+anti-CD47 therapy. versus the PC9 alone condition. However, drugs within this category exhibited different effects. Anthracyclines (FIG. 22B) and other chemotherapy drugs (FIG. 22C) exerted direct cytotoxicity to the PC9 cells alone, but the addition of macrophages+anti-CD47 protected the cancer cells from these drugs. In contrast, steroids (FIG. 22D) and retinoids (FIG. 22E) inhibited macrophage-mediated killing of the PC9 cells, thereby resulting in relatively enhanced growth in the macrophage+anti-CD47 condition.

FIGS. 23A-23B: Modeling additive growth reveals synergy between EGFR inhibitors and anti-CD47 therapy. FIG. 23A shows the estimated growth rates by fitting logistic growth models to time series data collected on PC9 cells grown under various conditions. Because the presence of macrophages with anti-CD47 antibodies elicits an anti-tumor response that limits the carrying capacity of PC9 cultures, the plots for fitted growth are rendered in terms of cell populations (i.e. area of GFP+ cells) normalized to carrying capacity. FIG. 23B shows how the estimated growth rates were used to construct a purely additive model (i.e., without any synergy or antagonism) for the combined effects of erlotinib and macrophages+anti-CD47 antibodies. The dashed lines for each curve indicate the standard errors in the fitting. The dramatic reduction observed in the growth rate of PC9 cells due to the combination of erlotinib with macrophages+anti-CD47 antibodies was far greater than that predicted under the assumption of additivity, indicating that cooperativity is present.

FIGS. 24A-24D: Analysis of apoptosis and cell death in response to targeted therapies. Lung cancer cells were treated with the indicated targeted therapies for 2-5 days. Adherent cells were collected and analyzed by flow cytometry for viability and apoptosis using annexin V and DAPI. FIG. 24A present representative plots showing PC9 cells treated with vehicle control or erlotinib to demonstrate gating strategy. Gates were drawn to quantify apoptotic cells (annexin V+, DAPI−) and dead cells (annexin V+, DAPI+). FIG. 24B shows the quantification of the percentage of PC9 cells undergoing apoptosis or cell death in response to the indicated EGFR TKIs. FIG. 24C shows the quantification of the percentage of NCI-H3122 cells undergoing apoptosis or cell death in response to the indicated ALK TKIs. FIG. 24D shows the quantification of the percentage of NCI-H358 cells undergoing apoptosis or cell death in response to the indicated KRAS^{G12C} inhibitors. For FIGS. 24B-24D, the data represent mean±SD from 3 replicates performed from one experiment. Ns, not significant, *p<0.05, **p<0.01, ***p<0.001, ****p<0.0001 by one-way ANOVA with Tukey's multiple comparisons test.

FIGS. 25A-25D: Representative images of long-term co-culture assays using GFP+ PC9 cells and human macrophages. GFP+ PC9 cells were combined with primary human macrophages and the indicated drug therapies in 384-well plates. Representative images are shown from macrophages derived from an individual blood donor at t=6 days and 12 hours. FIG. 25A shows the whole well imaging of the GFP+ channel from wells treated with the indicated therapies. FIG. 25B shows the image mask of GFP+ pixels used for quantification and analysis. FIG. 25C shows an overlay of GFP+ channel with phase contrast imaging. FIG. 25D presents phase contrast imaging showing the confluency of wells with GFP+ PC9 cells and primary human macrophages present.

FIGS. 26A-26D: Growth curves of long-term assays using human macrophages and different EGFR mutant lung cancer specimens. GFP+ lung cancer cells were combined with primary human macrophages and the indicated drug therapies in 384-well plates. The GFP+ area, representing the growth or death of the GFP+ cancer cells, was evaluated by whole-well imaging every 4 hours and quantified by automated image analysis. FIG. 26A shows how GFP+ PC9 cells co-cultured with macrophages and erlotinib (left). gefitinib (middle), or osimertinib (right). FIG. 26B shows co-culture assays using GFP+ PC9 cells and human macrophages to evaluate a dose-response relationship. The concentration of anti-CD47 was titrated alone or in combination with gefitinib at 100 nM. The IC₅₀ for anti-CD47 improved from 223.2 ng/ml (95% CI 158.2-317.3) to 71.25 ng/ml (95% CI 52.39-97.22). GFP+ area measured and compared on day 6.5 of co-culture. FIG. 26C shows how GFP+ MGH119 cells co-cultured with macrophages and erlotinib (left), gefitinib (middle), or osimertinib (right). FIG. 26D shows how GFP+ MGH134 cells co-cultured with macrophages and erlotinib (left), gefitinib (middle), or osimertinib (right). For FIGS. 26A-26D, the data at each timepoint represent mean±SEM from 3-4 technical replicates per donor and n=4-8 independent macrophage donors, ns, not significant, *p<0.05, **p<0.01, ***p<0.001, ****p<0.0001 by two-way ANOVA with Holm-Sidak multiple comparisons test on day 14 of co-culture or as indicated.

FIGS. 27A-27C: Growth curves of long-term assays using human macrophages and an ALK rearranged lung cancer cell line. GFP+ NCI-H3122 lung cancer cells were combined with primary human macrophages and the indicated drug therapies in 384-well plates. The GFP+ area, was evaluated by whole-well imaging every 4 hours and quantified by automated image analysis. GFP+ NCI-H3122 cells co-cultured with macrophages and crizotinib (FIG. 27A), alectinib (FIG. 27B), or lorlatinib (FIG. 27C). Data at each timepoint represent mean±SEM from 3 technical replicates per donor using n=4 independent macrophage donors. ****p<0.0001 by two-way ANOVA with Holm-Sidak multiple comparisons test on day 14 of co-culture.

FIGS. 28A-28D: Flow cytometry analysis of long-term co-cultures assays demonstrates phagocytosis and elimination of cancer cells. Primary human macrophages were co-cultured with GFP+ NCI-H358 cells (FIGS. 28A-28B) or GFP+ PC9 cells (FIGS. 28C-28D) and the indicated therapies. Cells were collected on day 4 of co-culture and analyzed by flow cytometry. Macrophages were identified by APC anti-CD45 and lung cancer cells were identified by GFP fluorescence. The percentage of GFP+ macrophages was quantified as a representation of phagocytosis. An increase in the percentage of CD45+ cells (FIGS. 28A and 28C) was observed due to elimination of cancer cells in the co-culture. Similarly, the percentage of phagocytic macrophages decreased in the combo therapy treatment due to decreases in cancer cell number and digestion of internalized material (FIGS. 28B and 28D). For each condition, representative FACS plots are shown on the left and summary bar graphs depicting mean±SD are shown on the right, ns, not significant, *p<0.05, ****p<0.0001 by two-way ANOVA with Tukey's multiple comparisons test from one experiment using macrophages derived from 3 independent donors with 8 technical replicates per donor (PC9) or 1 donor with 8 technical replicates (NCI-H358).

FIGS. 29A-29B: Analysis of macrophage polarization state following co-culture of macrophages and lung cancer cells. Primary human macrophages were cultured alone or with GFP+ PC9 cells and the indicated therapies. Cells were collected on day 4 of co-culture and analyzed by flow cytometry for the antigens associated with (FIG. 29A) M1 polarization (CD86, MHC II); or (FIG. 29B) M2 polarization (CD163, CD206). Bar graphs depict mean±SD from n=3 independent donors with one technical replicate per donor, ns, not significant, *p<0.01, ****p<0.0001 by two-way ANOVA with Tukey's multiple comparisons test from one independent experiment.

FIGS. 30A-30C: The combination of targeted therapies and anti-CD47 elicits unique cytokine and gene expression signatures in co-culture assays. FIG. 30A is a diagram showing the experimental setup of the cytokine and RNA profiling experiments. Primary human macrophages were co-cultured with GFP+ target NSCLC cells (PC9 or NCI-H358) with targeted therapies and/or an anti-CD47 antibody. Cells were co-cultured for 4-7 days. Supernatants were collected and subjected to multiplex cytokine analysis of 71 human analytes by addressable laser bead immunoassay. In a separate experiment, adherent cells were collected and subjected to targeted gene expression profiling of 770 myeloid-derived genes using an nCounter Myeloid Innate Immunity Panel (Nanostring). FIG. 30B shows the multiplex cytokine analysis of supernatants from co-culture assays using primary human macrophages and PC9 or NCI-H358 NSCLC cells. Each column represents data from the indicated drug treatments, and cytokines levels were compared by mean fluorescence intensity. From left to right, cell type for each column is PC9 (Vehicle), NCI-H358 (Vehicle), PC9 (Osimertinib), NCI-H358 (Sotorasib), NCI-H358 (Adagrasib), PC9 (Anti-CD47), NCI-H358 (Anti-CD47), PC9 (Osimertinib+Anti-CD47). NCI-H358 (Sotorasib+Anti-CD47), NCI-H358 (Adagrasib+Anti-CD47). Cytokines that were statistically significant by ANOVA (FDR<0.05) across experiments between anti-CD47 therapy and combo therapy groups are indicated in bold with an asterisk. The scale indicates logz fold-change versus mean for each individual cytokine, and the mean level for each cytokine is shown in the bar graph on the right. Data represent specimens collected on day 4 and day 7. with each time point containing 4 technical replicates from three independent donors mixed in equal ratios (PC9 experiment), or 1 technical replicate from each of 4 independent donors tested individually or mixed in equal ratios. FIG. 30C shows the targeted gene expression analysis depicting myeloid-derived genes from co-culture assays of primary human macrophages and NCI-H358 cells. The heatmap indicates hierarchical clustering of genes that were significantly downregulated or upregulated following treatment with the combination of sotorasib and anti-CD47 therapy versus all other treatment groups by ANOVA (FDR<0.05). The scale indicates log₂ fold-change versus mean for each individual gene. Data represent analysis performed with 4 independent donors with one technical replicate per donor with specimens collected on day 4 of co-culture. Genes associated with phagocytosis and/or cell-cell adhesion are VASP, ALCAM, ITGA5, ITGB2, ITGAM, ITGAX, HAVCR2, CD44, PPARG, ITGAL, CDKN1A, FCAR, and LAT. Genes that have reported proinflammatory functions are CXCL16, TREM2, OSCAR, C5AR1, ALOX5, S100A11, MAP3K14, CEBPB, RGS1, CCL5, IL17RA, CD40, CCL3, and MYD88.

FIGS. 31A-31C: Growth curves of lung cancer tumors in xenograft treatment experiments. Full growth curves from mouse xenograft tumor models shown in FIGS. 18A-18F, depicting tumor volumes as mean±SEM (left), growth curves from individual mice (middle) or fold-change from individual mice. FIG. 31A shows GFP+ MGH134-1 cells treated with vehicle control, anti-CD47 alone, osimertinib alone, or the combination (combo). FIG. 31B shows GFP+ NCI-H3122 cells treated with vehicle control, anti-CD47 alone, lorlatinib alone, or the combination. FIG. 31C shows GFP+ NCI-H358 cells treated with vehicle control, anti-CD47 alone, sotorasib alone, or the combination. For FIGS. 31A-31C, *p<0.05, **p<0.01, ***p<0.001 for the combination therapy versus targeted therapy by unpaired t test. For each experiment, n=4 mice per treatment cohort.

FIG. 32: Validation of a CD47 KO line generated by CRISPR/Cas9 editing of 3LL ΔNRAS cells. Cell-surface CD47 expression as detected by flow cytometry for wild-type (WT) 3LL ΔNRAS cells or a CD47 knockout (KO) variant. The data are depicted as mean±SD from three replicates (left), or as representative histograms (right), ns, not significant, **p<0.01 by one-way ANOVA with Holm-Sidak multiple comparisons test.

FIGS. 33A-33D: Proliferation of NSCLC cell lines in vitro after acquiring resistance to targeted therapies. Resistant cell lines were generated by prolonged culture of NSCLC cell lines in appropriate targeted therapy. Proliferation was evaluated by confluency analysis as measured by phase microscopy and automated image analysis. Proliferation was measured without drug selection or with 1 uM targeted therapy as indicated. Cell lines tested included PC9 cells resistant to gefitinib (FIG. 33A) or osimertinib (FIG. 33B), NCI-H3122 cells resistant to crizotinib (FIG. 33C), or NCI-H358 cells resistant to sotorasib (FIG. 33D). For the majority of cell lines, growth rates were comparable between parental and resistant cells in the absence of targeted therapy and approached 100% confluency by day 6.5 of culture. The data represent the mean of 3 technical replicates±SEM from one independent experiment for each cell line. For FIGS. 33A-33B, PC9 evaluation was performed in a single experiment and separated into distinct plots with the same parental curve reproduced for data visualization.

FIGS. 34A-34H: Changes in B2M and CD73 expression on lung cancer cells exposed to targeted therapies. FIG. 34A shows the downregulation of B2M on NCI-H358 cells or NCI-H3122 cells resistant to the indicated targeted therapies. FIG. 34B shows the downregulation of B2M on NCI-H3122 cells following treatment with the indicated ALK inhibitors. FIG. 34C shows how B2M was not downregulated on PC9 cells that were resistant to EGFR inhibitors, nor PC9 cells exposed to EGFR inhibitors in culture (FIG. 34D). FIG. 34E shows how CD73 is downregulated on NCI-H358 and PC9 cells that are resistant to the indicated targeted therapies. FIG. 34F shows how NCI-H3122 cells downregulate CD73 in response to the indicated targeted therapies. FIG. 34G shows how PC9 cells that are resistant to gefitinib did not downregulate CD73. FIG. 34H shows how CD73 is dynamically regulated on the surface of PC9 cells in response to EGFR inhibitors, with initial downregulation after 3 days of exposure, followed by increased surface expression. For FIGS. 34A-34H, the data represent mean±SD from 3 technical replicates from individual experiments, ns, not significant, ****p<0.0001 by one-way ANOVA with Holm-Sidak multiple comparisons test.

FIGS. 35A-35B: Validation of B2M KO and CD73 KO lines generated by CRISPR/Cas9 editing of human lung cancer cell lines. FIG. 35A shows the flow cytometry analysis of B2M expression on the surface of wild-type (WT) PC9. NCI-H358, MGH134, and MGH119 cells compared to their respective B2M KO variants. Left, quantification of geometric mean fluorescence intensity (Geo. MFI). Right, representative histograms showing B2M surface expression. The cell types listed on the right correspond vertically to the histogram (i.e., unstained is at the top of the histogram and PCM B2M KO is at the bottom of the histogram.). FIG. 35B shows the flow cytometry analysis of CD73 expression on the surface of wild-type (WT) PC9 and NCI-H358 cells compared to their respective CD73 KO variants. Left, quantification of geometric mean fluorescence intensity (Geo. MFI). Right, representative histograms showing B2M surface expression. The cell types listed on the right correspond vertically to the histogram (i.e., unstained is at the top of the histogram and PCM B2M KO is at the bottom of the histogram.). For FIGS. 35A-35B, the data represent mean±SD from 3 technical replicates from one individual experiment, ns, not significant, ****p<0.0001 by one-way ANOVA with Holm-Sidak multiple comparisons test.

FIGS. 36A-36C: Genetic deletion of B2M or CD73 does not make some NSCLC cell lines more vulnerable to macrophage attack. FIG. 36A shows the evaluation of wild-type versus B2M KO PC9 cells in long-term co-culture assays with human macrophages. Cells were treated with vehicle control or an anti-CD47 antibody. The data were combined from two independent experiments performed with a total of n=8 independent macrophage donors with 3 technical replicates per donor. FIGS. 36B-36C show the evaluation of wild-type versus CD73 KO NCI-H358 (FIG. 36B) or NCI-H3122 cells (FIG. 36C) in long-term co-culture assays with human macrophages. Cells were treated with vehicle control or an anti-CD47 antibody. The data represent the mean±SD from n=6independent macrophage donors with 3 technical replicates per donor from one experiment. For FIGS. 36A-36C, ns, not significant, *p<0.05, **p<0.01, ***p<0.001, ****p<0.0001 by two-way ANOVA with Holm-Sidak multiple comparisons test.

### DETAILED DESCRIPTION OF CERTAIN EMBODIMENTS

Provided herein are efficient and effective screening methods for identifying agents that modulate macrophage activity (e.g., activate or inhibit macrophage function). In turn, the agents identified may be therapeutic agents for a wide variety of disease (e.g., cancer). The assay methods provide improvements in studying macrophage function as the methods integrate all possible mechanisms of macrophage-mediated cytotoxicity (e.g., phagocytosis, direct cytotoxicity, and trogocytosis).

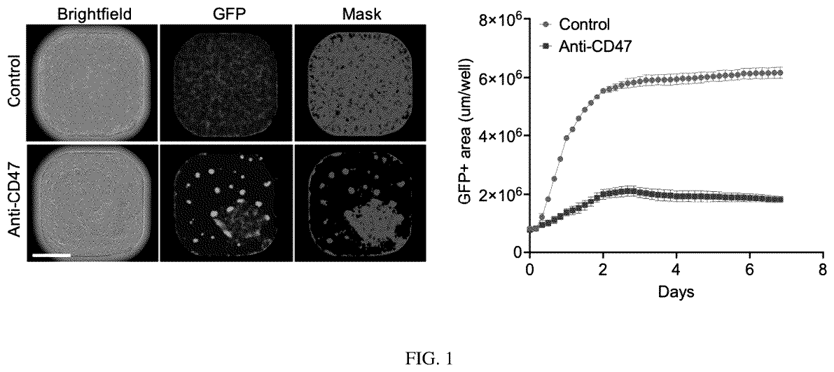

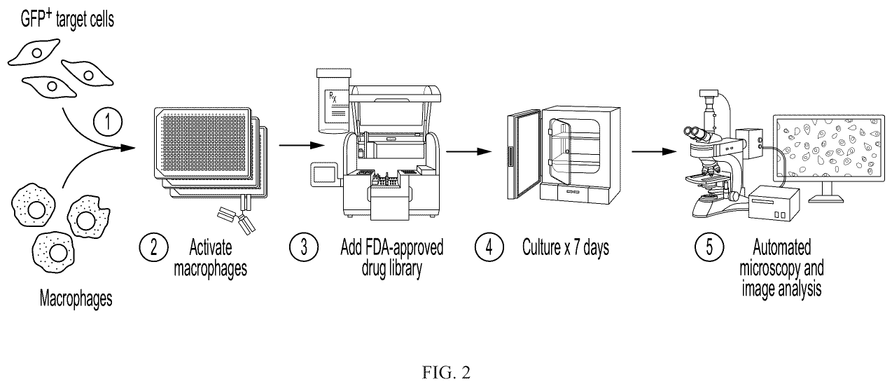

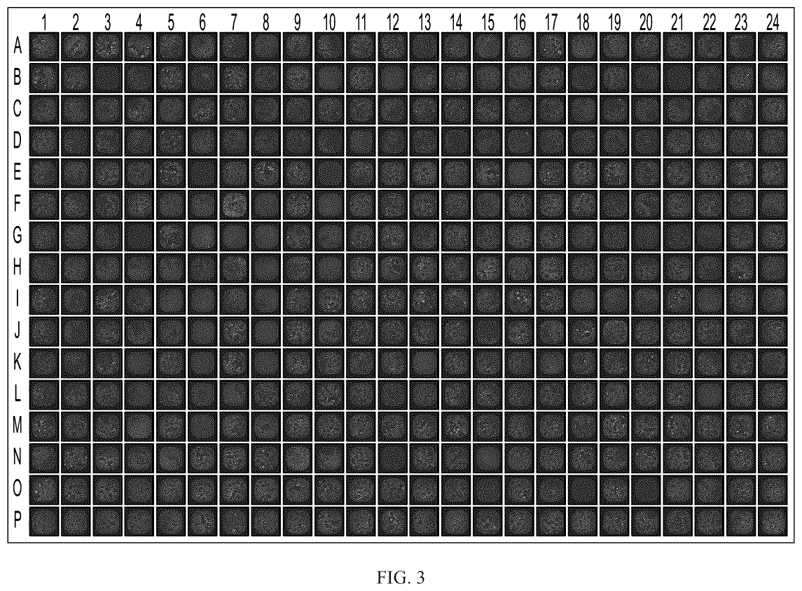

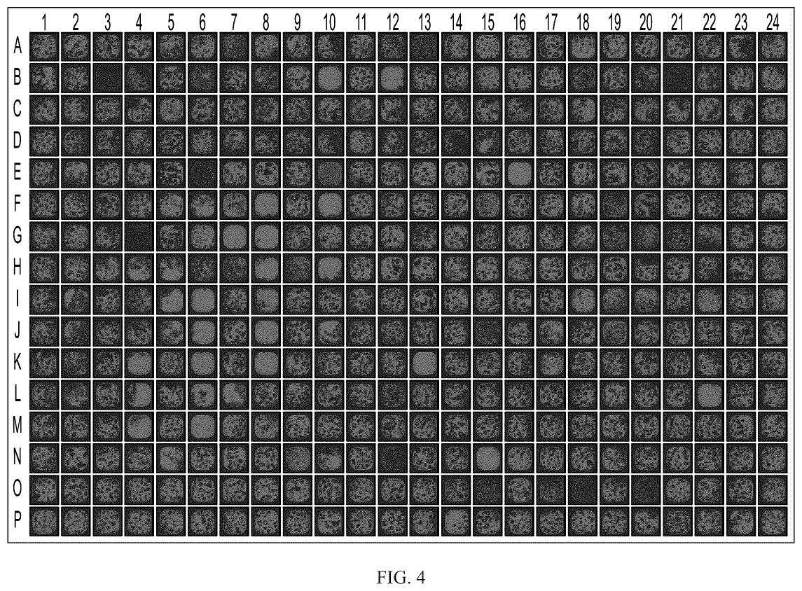

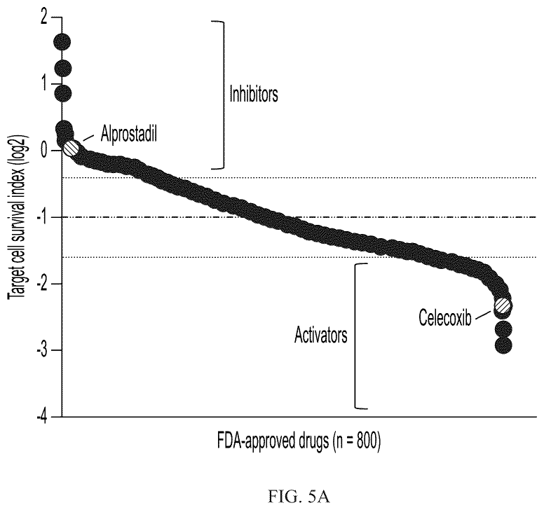

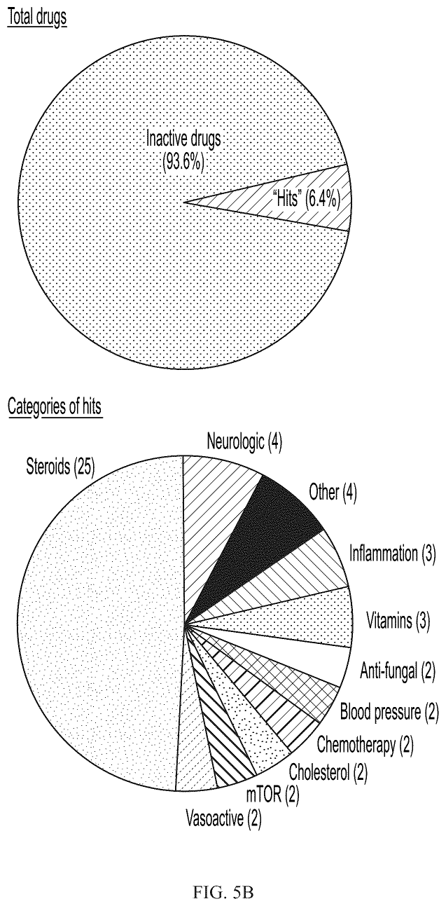

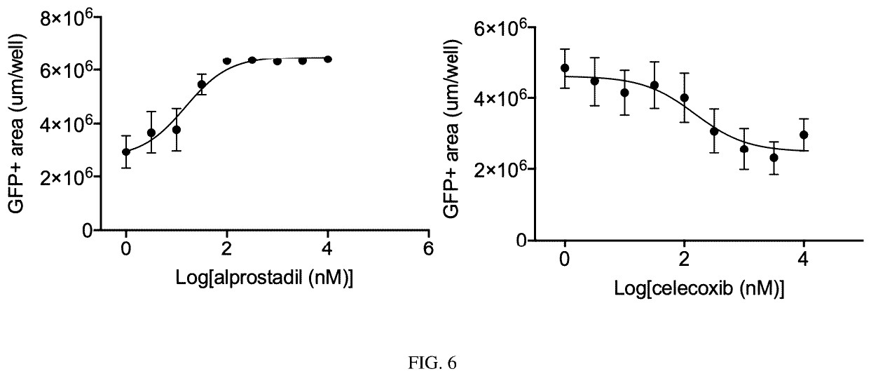

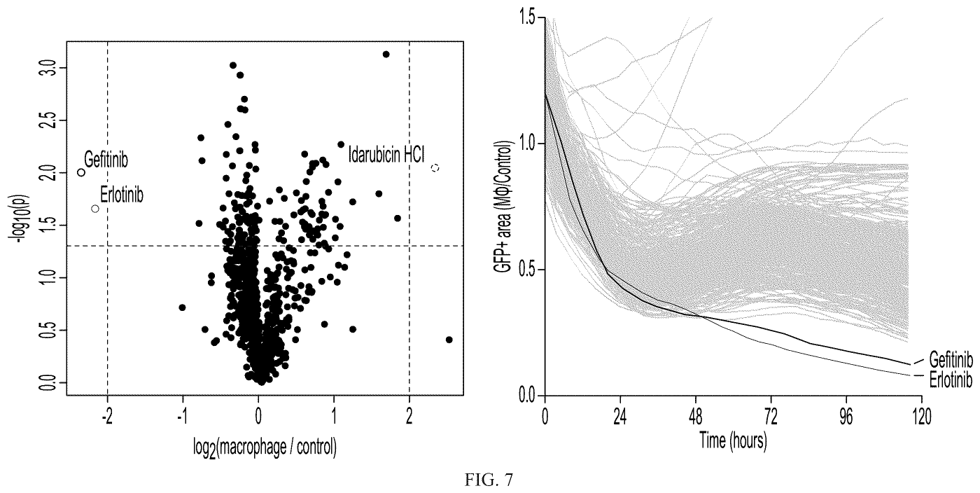

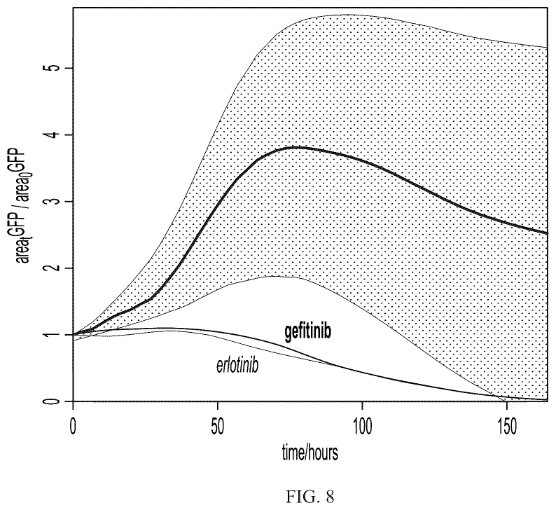

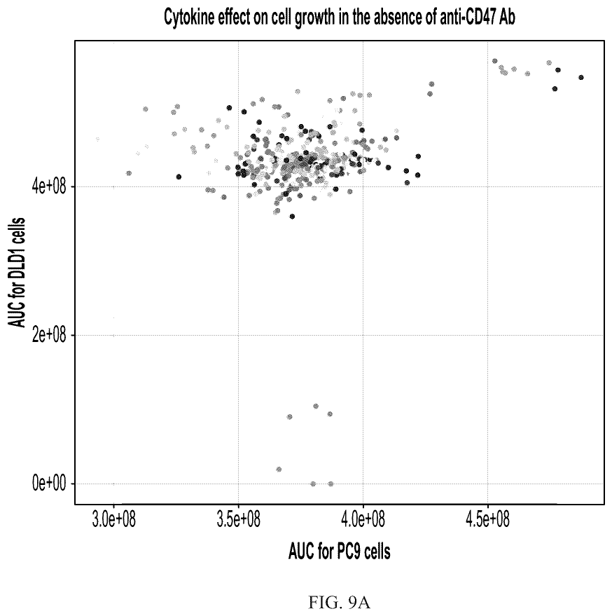

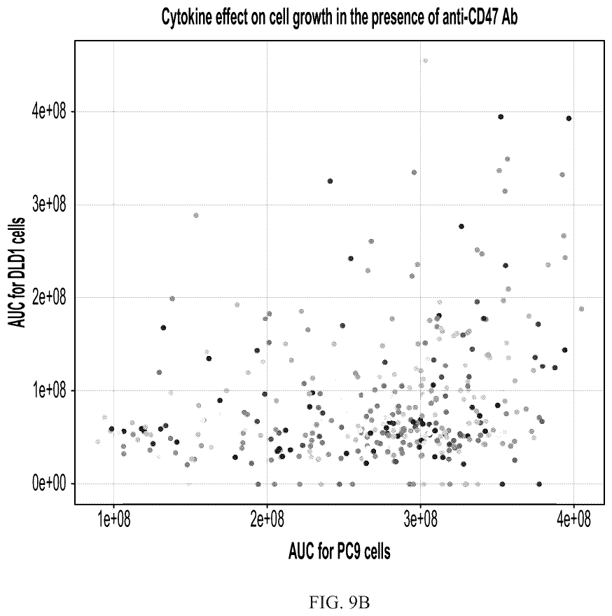

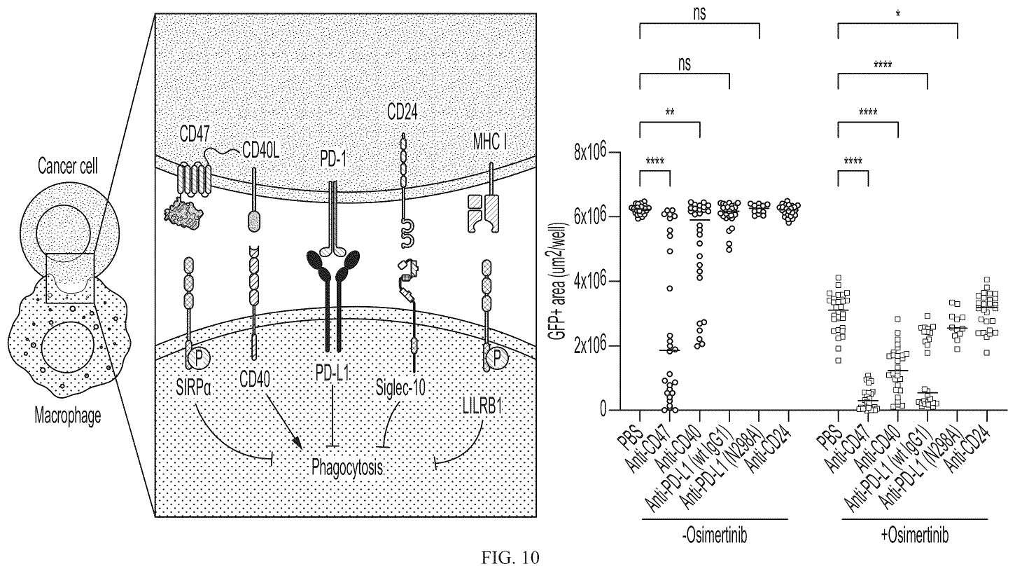

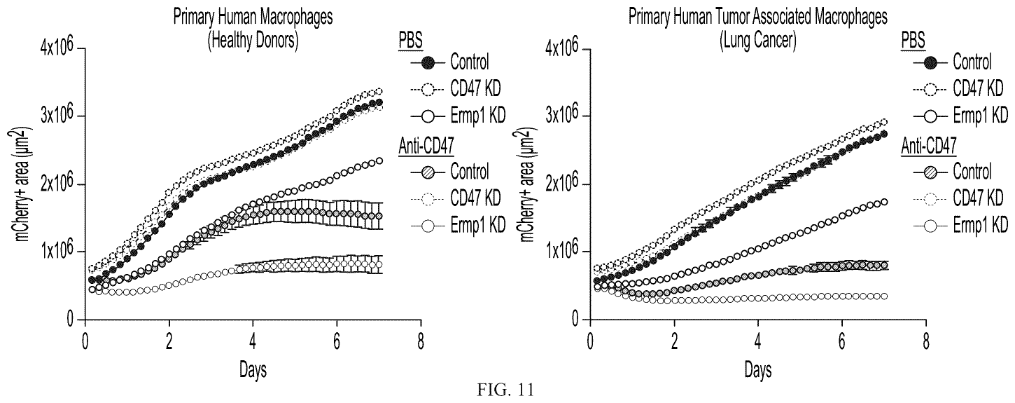

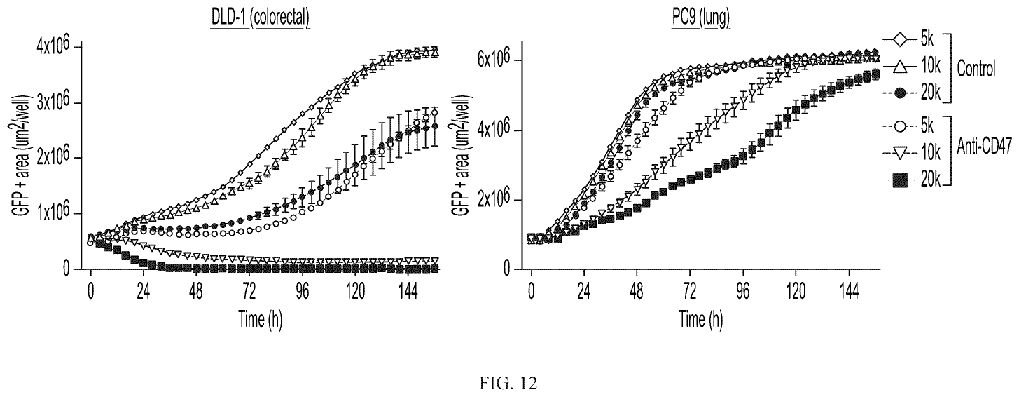

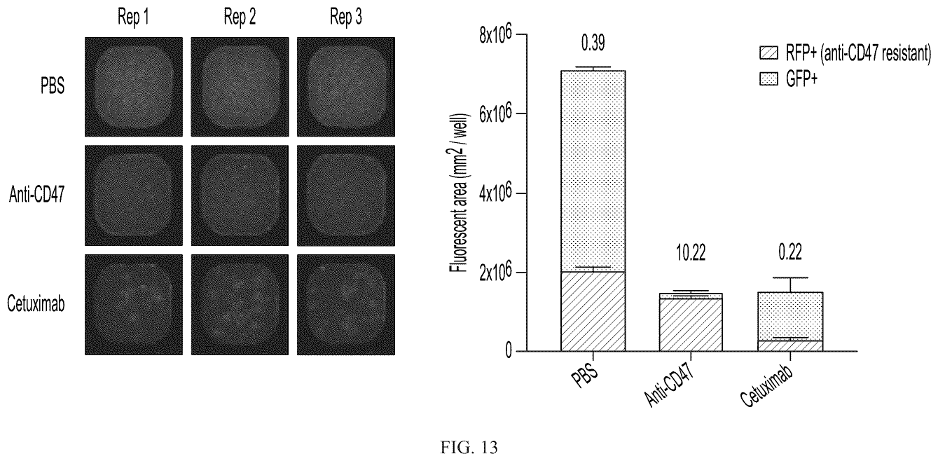

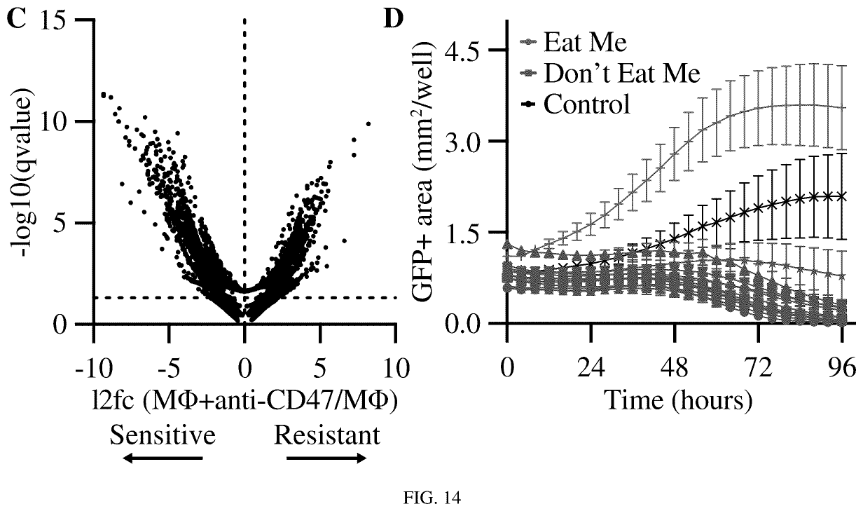

For example, to enable screening of compounds that promote macrophage anti-tumor function, a novel high throughput functional assay was developed to measure macrophage-mediated cytotoxicity of cancer cells. In the assay, primary human macrophages were combined with green fluorescent protein expressing (GFP+) cancer cells in 384-well plates. Different therapies were added and co-cultured for 7 days. Automated microscopy and image analysis were performed to quantify the growth of GFP+ cells over time (e.g., FIG. 1). The test characteristics of this assay were validated and confirmed that it can be run with a Z′ of 0.51 and CV 1.6%. A screen of 800 FDA-approved drugs was completed, which identified agents that activate or inhibit macrophage-mediated cytotoxicity, including some that act in coordinated pathways such as prostaglandin synthesis (FIGS. 5 and 6).

The screening methods described herein are further useful in screening for novel small molecules and biologics that act on macrophages, and thereby discovering therapies that take advantage of macrophage inhibition or activation of phagocytosis to treat a variety of disease.

### Methods of Identifying Agents That Modulate Macrophage Function

One aspect of the present disclosure relates to methods of identifying an agent that modulates macrophage function. In certain embodiments, the agent is an activator of macrophage function. In certain embodiments, the agent activates macrophages such that there is an increase in the amount and/or rate of phagocytosis achieved by the macrophages. In certain embodiments, the agent activates macrophages such that there is an increase in the amount and/or rate of cytotoxicity mediated by the macrophages. In certain embodiments, the agent promotes macrophage-mediated cytotoxicity. In certain embodiments, the agent activates macrophages such that there is an increase in the amount and/or rate of killing of target cells by the macrophages. In certain embodiments, the agent promotes the killing of target cells. In certain embodiments, the agent promotes macrophage anti-cancer function. In other embodiments, the agent is an inhibitor of macrophage function.

In certain embodiments, the present disclosure provides a method of identifying an agent that modulates macrophage function, the method comprising:
providing a population of macrophages and a population of target cells in an assay mixture;
adding a candidate agent to the assay mixture;
culturing the assay mixture;
determining the amount of growth of the population of target cells in the assay mixture at one or more timepoints; and
comparing the amount of growth of the population of target cells in the assay mixture with the amount of growth of a population of the same type of target cells in a control mixture cultured without the candidate agent at the same timepoint;
wherein the candidate agent is determined to be an agent that modulates macrophage function when the amount of growth of the population of target cells in the assay mixture is different from the amount of growth of the population of target cells in the control mixture at the same timepoint.

In another aspect, the present disclosure provides a method of identifying an agent that promotes macrophage-mediated cytotoxicity, the method comprising:
providing a population of macrophages and a population of target cells in an assay mixture;
adding a candidate agent to the assay mixture;
culturing the assay mixture;
determining the amount of growth of the population of target cells in the assay mixture at one or more timepoints; and
comparing the amount of growth of the population of target cells in the assay mixture with the amount of growth of a population of the same type of target cells in a control mixture cultured without the candidate agent, at the same timepoint;
wherein the candidate agent is determined to be an agent that promotes macrophage-mediated cytotoxicity when the amount of growth of the population of target cells in the assay mixture is less than the amount of growth of the population of target cells in the control mixture at the same timepoint.

In another aspect, the present disclosure provides a method of identifying an agent that promotes macrophage anti-cancer function, the method comprising:
providing a population of macrophages and a population of target cells in an assay mixture;
adding a candidate agent to the assay mixture;
culturing the assay mixture;
determining the amount of growth of the population of target cells in the assay mixture at one or more timepoints; and
comparing the amount of growth of the population of target cells in the assay mixture with the amount of growth of a population of the same type of target cells in a control mixture cultured without the candidate agent at the same timepoint;
wherein the candidate agent is determined to be an agent that promotes macrophage anti-cancer function when the amount of growth of the population of target cancer cells in the assay mixture is lower than the amount of growth of the population of target cancer cells in the control mixture at the same timepoint.

In another aspect, the present disclosure provides a method of identifying a cancer therapy, the method comprising:
providing a population of macrophages and a population of target cells in an assay mixture;
adding a candidate agent to the assay mixture;
culturing the assay mixture;
determining the amount of growth of the population of target cells in the assay mixture at one or more timepoints; and
comparing the amount of growth of the population of target cells in the assay mixture with the amount of growth of a population of the same type of target cells in a control mixture cultured without the candidate agent at the same timepoint;
wherein the candidate agent is determined to be a cancer therapy when the amount of growth of the population of target cancer cells in the assay mixture is lower than the amount of growth of the population of target cancer cells in the control mixture at the same timepoint.

### Selective Modulation of Macrophage Function for a Target Cell

In another aspect, the methods described herein further comprise providing, in the assay mixture, a population of second target cells different than the population of target cells;
determining the amount of growth of the population of second target cells in the assay mixture at one or more timepoints; and
comparing the amount of growth of the second population of target cells in the assay mixture with the amount of growth of a population of second target cells in a control mixture cultured without the candidate agent at the same timepoint;
wherein the candidate agent is determined to be an agent that promotes macrophage-mediated cytotoxicity when the amount of growth of the population of second target cells in the assay mixture is less than the amount of growth of the population of second target cells in the control mixture at the same timepoint

By adding a population of second target cells, the assay can be multiplexed with two different types of target cells (e.g., a GFP+ cell line versus and RFP+ target cell line) to double the amount of information per well and to understand how macrophages prefer one target versus another. This greatly increases the amount of information that can be obtained. In one embodiment, the population of first target cells is a cell line that is resistant to anti-CD47 therapy and the population of second target cells is a wild-type line. Such an assay can be used to screen agents that might selectively work to enhance killing of wild-type versus resistant cell-lines.

Thus, in this aspect provided is a method of identifying an agent that selectively modulates macrophage function for a target cell, the method comprising:
providing a population of macrophages, a population of first target cells, and a population of second target cells in an assay mixture, wherein the first and second target cells are different;
adding a candidate agent to the assay mixture;
culturing the assay mixture;
determining the amount of growth of the populations of first and second target cells in the assay mixture at one or more timepoints;
comparing the amount of growth of the population of first target cells in the assay mixture with the amount of growth of a population of the first target cells in a control mixture cultured without the candidate agent, at the same timepoint;
comparing the amount of growth of the population of second target cells in the assay mixture with the amount of growth of a population of the second target cells in a control mixture cultured without the candidate agent, at the same timepoint;
wherein the candidate agent is determined to be an agent that selectively modulates macrophage function for a target cell when the amount of growth of the population of first target cells in the assay mixture compared to the amount of growth in the control mixture is different than the amount of growth of the population of second target cells in the assay mixture compared to the amount of growth in the control mixture at the same timepoint.

In certain embodiments, the difference is at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 80%, at least 95%, or at least 99%.

In certain embodiments, the agent selectively inhibits macrophage function for a target cell. In certain embodiments, the agent selectively activates macrophage function for a target cell. In certain embodiments, the agent selectively promotes macrophage-mediated cytotoxicity of a target cell. In certain embodiments, the agent selectively promotes macrophage anti-cancer function for a target cancer cell.

### Macrophages

In certain embodiments, the methods described herein further comprise activating the population of macrophages. In certain embodiments, the methods described herein further comprise activating the population of macrophages by adding a macrophage-directed immunotherapy to the assay mixture prior to adding the candidate agent to the assay mixture.

A macrophage-directed immunotherapy is an immunotherapy that derives its therapeutic effect by stimulating macrophages. Such stimulation can mobilize macrophage and myeloid components to destroy a tumor and its stroma, including the tumor vasculature. Macrophages can be induced to secrete antitumor cytokines and/or to perform phagocytosis, including antibody-dependent cellular phagocytosis.

In certain embodiments, the macrophage-directed immunotherapy is an immunotherapeutic agent. In certain embodiments, the macrophage-directed immunotherapy is a macrophage immune checkpoint inhibitor. In certain embodiments, the immunotherapeutic agent is a macrophage immune checkpoint inhibitor. A macrophage immune checkpoint inhibitor functions to stimulate macrophage phagocytosis of cancer cells. For example. CD47 is associated with a macrophage immune checkpoint (CD47/SIRPα as described herein).

In certain embodiments, the macrophage-directed immunotherapy is a small molecule. In certain embodiments, the macrophage-directed immunotherapy is a biologic. In certain embodiments, the biologic is a protein. In certain embodiments, the biologic is an antibody or fragment thereof. In certain embodiments, the biologic is a nucleic acid that encodes a protein. In certain embodiments, the biologic is a cytokine

In certain embodiments, the macrophage-directed immunotherapy comprises modulation of CD47, SIRPα, MHC I, B2M, CD24, CALR, CD40, PD-L1, APMAP, GPR84, VCAM1, CD11b, SIGLEC-10, PD-L1, PD-L2, PD-1, CD73, Galectin-9, CD14, CD80, CD86, SIRPb, SIRPg, SLAMF7, MARCO, AXL, CLEVER-1, ILT4, TIM-3, TIM-4, LRP-1, calreticulin, TREM1, TREM2, GD2, FcgRI, FcgRIIa, FcgRIIb, FcgRIII, MUC1, CD44, CD63, CD36, CD84, CD164, CD82, CD18, SIGLEC-7, CD166, CD39, CD46, LILRA1, LILRA2 (ILT1), LILRA3 (ILT6), LILRA4 (ILT7), LILRB1 (ILT2), LILRB2 (ILT4), LILRB3 (ILT5), LILRB4 (ILT3), LILRB5, CD85b (ILT8 or ILT9), CD85m (ILT10), CD85f (ILT11), CD276, CD88, CD99, PILRa, Siglec-9, CD206, CD163, CD84 (SLAMF5), C3aR, or CLEC12A. In certain embodiments, the macrophage-directed immunotherapy comprises modulation of CD47, SIRPα, MHC I, CD24, CALR, CD40, PD-L1, APMAP, GPR84, VCAM1, CD11b, SIGLEC-10, PD-L1, PD-L2, PD-1, CD73, Galectin-9, CD14, CD80, CD86, SIRPb, SIRPg, SLAMF7, MARCO, AXL, CLEVER-1, ILT4, TIM-3, TIM-4, LRP-1, calreticulin, TREM1, TREM2, GD2, FcgRI, FcgRIIa, FcgRIIb, FcgRIII, MUC1, CD44, CD63, CD36, CD84, CD164, CD82, CD18, SIGLEC-7, CD166, CD39, CD46, LILRA1, LILRA2 (ILT1), LILRA3 (ILT6), LILRA4 (ILT7), LILRB1 (ILT2), LILRB2 (ILT4), LILRB3 (ILT5), LILRB4 (ILT3), LILRB5, CD85b (ILT8 or ILT9), CD85m (ILT10), CD85f (ILT11), CD276, CD88, CD99, PILRa, Siglec-9, CD206, CD163, CD84 (SLAMF5), C3aR, or CLEC12A.

In certain embodiments, the macrophage-directed immunotherapy comprises modulation of CD47, SIRPα, MHC I, CD24, CALR, CD40, PD-L1, APMAP, GPR84, VCAM1, or CD11b. In certain embodiments, the macrophage-directed immunotherapy comprises modulation of CD47, SIRPα, MHC I, CD24, CALR, CD40, PD-L1, APMAP, GPR84, VCAM1, CD11b, B2M, or CD73. In certain embodiments, the macrophage-directed immunotherapy comprises modulation of CD47. In certain embodiments, the macrophage-directed immunotherapy comprises modulation of B2M. In certain embodiments, the macrophage-directed immunotherapy comprises modulation of CD73.

In certain embodiments, the macrophage-directed immunotherapy is a CD47 inhibitor, a SIRPα inhibitor, an MHC I inhibitor, a B2M inhibitor, a CD24 inhibitor, a CALR inhibitor, a CD40 agonist, a PD-L1 inhibitor, an APMAP inhibitor, a GPR84 inhibitor, a VCAM1 inhibitor, a CD11b inhibitor, a SIGLEC-10 inhibitor, a PD-L2 inhibitor, a PD-1 inhibitor, a CD73 inhibitor, a Galectin-9 inhibitor, a CD14 inhibitor, a CD80 inhibitor, a CD86 inhibitor, a SIRPb inhibitor, a SIRPg inhibitor, a SLAMF7 inhibitor, a MARCO inhibitor, an AXL inhibitor, a CLEVER-1 inhibitor, an ILT4 inhibitor, a TIM-3 inhibitor, a TIM-4 inhibitor, an LRP-1 inhibitor, a calreticulin inhibitor, a TREM1 inhibitor, a TREM2 inhibitor, a GD2 inhibitor, an FcgRI inhibitor, an FcgRIIa inhibitor, an FcgRIIb inhibitor, an FcgRIII inhibitor, a MUC1 inhibitor, a CD44 inhibitor, a CD63 inhibitor, a CD36 inhibitor, a CD84 inhibitor, a CD164 inhibitor, a CD82 inhibitor, a CD18 inhibitor, a SIGLEC-7 inhibitor, a CD166 inhibitor, a CD39 inhibitor, a CD46 inhibitor, an LILRA1 inhibitor, an LILRA2 inhibitor, an LILRA3 inhibitor, an LILRA4 inhibitor, an LILRB1 inhibitor, an LILRB2 inhibitor, an LILRB3 inhibitor, an LILRB4 inhibitor, an LILRB5 inhibitor, a CD85b inhibitor, a CD85m inhibitor, a CD85f inhibitor, a CD276 inhibitor, a CD88 inhibitor, a CD99 inhibitor, a PILRa inhibitor, a Siglec-9 inhibitor, a CD206 inhibitor, a CD163 inhibitor, a CD84 inhibitor, a C3aR inhibitor, or a CLEC12A inhibitor. In certain embodiments, the macrophage-directed immunotherapy is a CD47 inhibitor, a SIRPα inhibitor, an MHC I inhibitor, a CD24 inhibitor, a CALR inhibitor, a CD40 agonist, a PD-L1 inhibitor, an APMAP inhibitor, a GPR84 inhibitor, a VCAM1 inhibitor, a CD11b inhibitor, a SIGLEC-10 inhibitor, a PD-L2 inhibitor, a PD-1 inhibitor, a CD73 inhibitor, a Galectin-9 inhibitor, a CD14 inhibitor, a CD80 inhibitor, a CD86 inhibitor, a SIRPb inhibitor, a SIRPg inhibitor, a SLAMF7 inhibitor, a MARCO inhibitor, an AXL inhibitor, a CLEVER-1 inhibitor, an ILT4 inhibitor, a TIM-3 inhibitor, a TIM-4 inhibitor, an LRP-1 inhibitor, a calreticulin inhibitor, a TREM1 inhibitor, a TREM2 inhibitor, a GD2 inhibitor, an FcgRI inhibitor, an FcgRIIa inhibitor, an FegRIIb inhibitor, an FcgRIII inhibitor, a MUC1 inhibitor, a CD44 inhibitor, a CD63 inhibitor, a CD36 inhibitor, a CD84 inhibitor, a CD164 inhibitor, a CD82 inhibitor, a CD18 inhibitor, a SIGLEC-7 inhibitor, a CD166 inhibitor, a CD39 inhibitor, a CD46 inhibitor, an LILRA1 inhibitor, an LILRA2 inhibitor, an LILRA3 inhibitor, an LILRA4 inhibitor, an LILRB1 inhibitor, an LILRB2 inhibitor, an LILRB3 inhibitor, an LILRB4 inhibitor, an LILRB5 inhibitor, a CD85b inhibitor, a CD85m inhibitor, a CD85f inhibitor, a CD276 inhibitor, a CD88 inhibitor, a CD99 inhibitor, a PILRa inhibitor, a Siglec-9 inhibitor, a CD206 inhibitor, a CD163 inhibitor, a CD84 inhibitor, a C3aR inhibitor, or a CLEC12A inhibitor.

In certain embodiments, the macrophage-directed immunotherapy is a CD47 inhibitor, a SIRPα inhibitor, an MHC I inhibitor, a CD24 inhibitor, a CALR inhibitor, a CD40 agonist, a PD-L1 inhibitor, an APMAP inhibitor, a GPR84 inhibitor, a VCAM1 inhibitor, or a CD11b inhibitor. In certain embodiments, the macrophage-directed immunotherapy is a CD47 inhibitor, a SIRPα inhibitor, an MHC I inhibitor, a CD24 inhibitor, a CALR inhibitor, a CD40 agonist, a PD-L1 inhibitor, an APMAP inhibitor, a GPR84 inhibitor, a VCAM1 inhibitor, a CD11b inhibitor, a B2M inhibitor, or a CD73 inhibitor.

In certain embodiments, the macrophage-directed immunotherapy is a CD47 inhibitor or a SIRPα inhibitor. In certain embodiments, the macrophage-directed immunotherapy is a CD47 inhibitor and a SIRPα inhibitor. In certain embodiments, the macrophage-directed immunotherapy is a CD47 inhibitor. In certain embodiments, the macrophage-directed immunotherapy is a SIRPα inhibitor. In certain embodiments, the macrophage-directed immunotherapy is a B2M inhibitor. In certain embodiments, the macrophage-directed immunotherapy is a CD73 inhibitor.

In certain embodiments, the macrophage-directed immunotherapy is an anti-CD47 antibody, a SIRPα-Fc fusion protein, an anti-SIRPα antibody, an anti-MHC I antibody, an anti-CD24 antibody, an anti-CALR antibody, an anti-CD40 antibody, an anti-PD-L1 antibody, an anti-APMAP antibody, an anti-GPR84 antibody, an anti-VCAM1 antibody, an anti-CD11b antibody, an anti-SIGLEC-10 antibody, an anti-PD-L2 antibody, an anti-PD-1 antibody, an anti-B2M antibody, an anti-CD73 antibody, an anti-Galectin-9 antibody, an anti-CD14 antibody, an anti-CD80 antibody, an anti-CD86 antibody, an anti-SIRPb antibody, an anti-SIRPg antibody, an anti-SLAMF7 antibody, an anti-MARCO antibody, an anti-AXL antibody, an anti-CLEVER-1 antibody, an anti-ILT4 antibody, an anti-TIM-3 antibody, an anti-TIM-4 antibody, an anti-LRP-1 antibody, an anti-calreticulin antibody, an anti-TREM1 antibody, an anti-TREM2 antibody, an anti-GD2 antibody, an anti-FcgRI antibody, an anti-FcgRIIa antibody, an anti-FcgRIIb antibody, an anti-FcgRIII antibody, an anti-MUC1 antibody, an anti-CD44 antibody, an anti-CD63 antibody, an anti-CD36 antibody, an anti-CD84 antibody, an anti-CD164 antibody, an anti-CD82 antibody, an anti-CD18 antibody, an anti-SIGLEC-7 antibody, an anti-CD166 antibody, an anti-CD39 antibody, an anti-CD46 antibody, an anti-LILRA1 antibody, an anti-LILRA2 antibody, an anti-LILRA3 antibody, an anti-LILRA4 antibody, an anti-LILRB1 antibody, an anti-LILRB2 antibody, an anti-LILRB3 antibody, an anti-LILRB4 antibody, an anti-LILRB5 antibody, an anti-CD85b antibody, an anti-CD85m antibody, an anti-CD85f antibody, an anti-CD276 antibody, an anti-CD88 antibody, an anti-CD99 antibody, an anti-PILRa antibody, an anti-Siglec-9 antibody, an anti-CD206 antibody, an anti-CD163 antibody, an anti-CD84 antibody, an anti-C3aR antibody, or an anti-CLEC12A antibody. In certain embodiments, the macrophage-directed immunotherapy is an anti-CD47 antibody, a SIRPα-Fc fusion protein, an anti-SIRPα antibody, an anti-MHC I antibody, an anti-CD24 antibody, an anti-CALR antibody, an anti-CD40 antibody, an anti-PD-L1 antibody, an anti-APMAP antibody, an anti-GPR84 antibody, an anti-VCAMI antibody, an anti-CD11b antibody, an anti-SIGLEC-10 antibody, an anti-PD-L2 antibody, an anti-PD-1 antibody, an anti-CD73 antibody, an anti-Galectin-9 antibody, an anti-CD14 antibody, an anti-CD80 antibody, an anti-CD86 antibody, an anti-SIRPb antibody, an anti-SIRPg antibody, an anti-SLAMF7 antibody, an anti-MARCO antibody, an anti-AXL antibody, an anti-CLEVER-1 antibody, an anti-ILT4 antibody, an anti-TIM-3 antibody, an anti-TIM-4 antibody, an anti-LRP-1 antibody, an anti-calreticulin antibody, an anti-TREM1 antibody, an anti-TREM2 antibody, an anti-GD2 antibody, an anti-FcgRI antibody, an anti-FcgRIIa antibody, an anti-FcgRIIb antibody, an anti-FcgRIII antibody, an anti-MUC1 antibody, an anti-CD44 antibody, an anti-CD63 antibody, an anti-CD36 antibody, an anti-CD84 antibody, an anti-CD164 antibody, an anti-CD82 antibody, an anti-CD18 antibody, an anti-SIGLEC-7 antibody, an anti-CD166 antibody, an anti-CD39 antibody, an anti-CD46 antibody, an anti-LILRA1 antibody, an anti-LILRA2 antibody, an anti-LILRA3 antibody, an anti-LILRA4 antibody, an anti-LILRB1 antibody, an anti-LILRB2 antibody, an anti-LILRB3 antibody, an anti-LILRB4 antibody, an anti-LILRB5 antibody, an anti-CD85b antibody, an anti-CD85m antibody, an anti-CD85f antibody, an anti-CD276 antibody, an anti-CD88 antibody, an anti-CD99 antibody, an anti-PILRa antibody, an anti-Siglec-9 antibody, an anti-CD206 antibody, an anti-CD163 antibody, an anti-CD84 antibody, an anti-C3aR antibody, or an anti-CLEC12A antibody.

In certain embodiments, the macrophage-directed immunotherapy is an anti-CD47 antibody, an anti-SIRPα antibody, a SIRPα-Fc fusion protein, an anti-MHC I antibody, an anti-CD24 antibody, an anti-CALR antibody, an anti-CD40 antibody, an anti-PD-L1 antibody, an anti-APMAP antibody, an anti-GPR84 antibody, an anti-VCAMI antibody, an anti-CD11b antibody, an anti-CD73 antibody, or an anti-B2M antibody. In certain embodiments, the macrophage-directed immunotherapy is an anti-CD47 antibody, an anti-SIRPα antibody, a SIRPα-Fc fusion protein, an anti-MHC I antibody, an anti-CD24 antibody, an anti-CALR antibody, an anti-CD40 antibody, an anti-PD-L1 antibody, an anti-APMAP antibody, an anti-GPR84 antibody, an anti-VCAM1 antibody, or an anti-CD11b antibody. In certain embodiments, the macrophage-directed immunotherapy is an anti-CD47 antibody, a SIRPα-Fc fusion protein, or an anti-SIRPα antibody. In certain embodiments, the macrophage-directed immunotherapy is an anti-CD47 antibody or an anti-SIRPα antibody. In certain embodiments, the macrophage-directed immunotherapy is a SIRPα-Fc fusion protein. In certain embodiments, the macrophage-directed immunotherapy is an anti-SIRPα antibody. In certain embodiments, the macrophage-directed immunotherapy is an anti-CD47 antibody. In certain embodiments, the macrophage-directed immunotherapy is an anti-CD73 antibody. In certain embodiments, the macrophage-directed immunotherapy is an anti-B2M antibody.

In certain embodiments, the macrophage-directed immunotherapy is magrolimab, TTI-621, TTI-622, AO-176, HX-009, AK117, AK112, CC90002, STI-6643, PF-07257876, IMC-002, CPO107, SRF231, IBI188, IBI322, IMM2902, BAT7104, TG-1801, SL-172154, BI 765063, TQB2928, or GS-0189.

In certain embodiments, the macrophage-directed immunotherapy is magrolimab. In certain embodiments, the macrophage-directed immunotherapy is TTI-621. In certain embodiments, the macrophage-directed immunotherapy is TTI-622. In certain embodiments, the macrophage-directed immunotherapy is AO-176. In certain embodiments, the macrophage-directed immunotherapy is HX-009. In certain embodiments, the macrophage-directed immunotherapy is AK117. In certain embodiments, the macrophage-directed immunotherapy is AK112. In certain embodiments, the macrophage-directed immunotherapy is CC90002. In certain embodiments, the macrophage-directed immunotherapy is STI-6643. In certain embodiments, the macrophage-directed immunotherapy is PF-07257876. In certain embodiments, the macrophage-directed immunotherapy is TQB2928. In certain embodiments, the macrophage-directed immunotherapy is IMC-002. In certain embodiments, the macrophage-directed immunotherapy is CPO107. In certain embodiments, the macrophage-directed immunotherapy is SRF231. In certain embodiments, the macrophage-directed immunotherapy is IBI188. In certain embodiments, the macrophage-directed immunotherapy is IBI322. In certain embodiments, the macrophage-directed immunotherapy is IMM2902. In certain embodiments, the macrophage-directed immunotherapy is BAT7104. In certain embodiments, the macrophage-directed immunotherapy is TG-1801. In certain embodiments, the macrophage-directed immunotherapy is SL-172154. In certain embodiments, the macrophage-directed immunotherapy is BI 765063. In certain embodiments, the macrophage-directed immunotherapy is GS-0189.

In certain embodiments, the population of macrophages comprises macrophages isolated directly from a subject, as defined herein. In certain embodiments, the population of macrophages comprises mammalian macrophages (e.g., mice, monkey, dog, cats, human, and transgenically modified mammal macrophages). In certain embodiments, the population of macrophages comprises primary human macrophages. In certain embodiments, the population of macrophages comprises primary human tumor associated macrophages. In certain embodiments, the population of macrophages comprises macrophages derived from stem cells. In certain embodiments, the population of macrophages comprises macrophages derived from embryonic stem cells. In certain embodiments, the population of macrophages comprises macrophages derived from mammalian stem cells. In certain embodiments, the population of macrophages comprises macrophages derived from embryonic mammalian stem cells. In certain embodiments, the population of macrophages comprises genetically altered populations of macrophages.

In certain embodiments, the population of macrophages comprises at least 1,000, at least 2,000, at least 3,000, at least 4,000, at least 5,000, at least 6,000, at least 7,000, at least 8,000, at least 9,000, at least 10,000 at least 11,000, at least 12,000, at least 13,000, at least 14,000, at least 15,000, at least 16,000, at least 17,000, at least 18,000, at least 19.000, or at least 20,000 macrophages. In certain embodiments, the population of macrophages comprises about 1,000, about 2,000, about 3,000, about 4,000, about 5,000, about 6,000, about 7,000, about 8,000, about 9,000, about 10,000 about 11,000, about 12,000, about 13,000, about 14,000, about 15,000, about 16,000, about 17,000, about 18,000, about 19,000, or about 20,000 macrophages. In certain embodiments, the population of macrophages comprises about 1,000 to about 20,000 macrophages. In certain embodiments, the population of macrophages comprises about 5,000 to about 20,000 macrophages. In certain embodiments, the population of macrophages comprises at least 5,000 to about 20,000 macrophages. In certain embodiments, the population of macrophages comprises about 5,000 to about 15,000 macrophages. In certain embodiments, the population of macrophages comprises about 10,000 to about 20,000 macrophages. In certain embodiments, the population of macrophages comprises about 8,000 to about 12,000 macrophages. In certain embodiments, the population of macrophages comprises about 9,000 to about 11,000 macrophages. In certain embodiments, the population of macrophages comprises about 10,000 macrophages.

### Target Cells

As described herein, the target cells are any type of cell. In certain embodiments, the assay comprises multiple types of target cells, e.g., when identifying an agent that activates macrophages to selectively modulate macrophage function for target cells.

In certain embodiments, the target cell is a naturally occurring cell. In certain embodiments, the target cell is a genetically engineered cell line. In certain embodiments, the target cell is derived from a transgenic animal. In certain embodiments, the target cell is a bacterial, fungal, plant, or animal cell. In certain embodiments, the target cell is a mammalian cell. In certain embodiments, the target cell is a human cell. In certain embodiments, the target cells are collected from a subject (e.g., a human) through a medical procedure such as a biopsy. In certain embodiments, the target cells are collected from a subject (e.g., a human) and are part of a sample that is conditioned media, patient fluid, or any liquid substance that lacks or contains solutes. In certain embodiments, the target cells are part of a sample that is conditioned media, patient fluid, or any liquid substance that lacks or contains solutes. In certain embodiments, the assay mixture is collected from a subject (e.g., a human) through a medical procedure. In certain embodiments, the target cell is cultured (e.g., a culture derived from a complex population or a culture derived from a single cell type where the cells have differentiated into multiple lineages). In certain embodiments, the target cell is a healthy cell.

In certain embodiments, the target cell is resistant to phagocytosis.

In certain embodiments, the target cell is a disease associated cell. In certain embodiments, the target cell is a cell associated with autoimmune disease, infectious diseases, cardiovascular disease, hematological diseases, diseases of the central nervous system, metabolic disorders, or cancer. In certain embodiments, the target cell is a cancer cell. In certain embodiments, the target cell is a cancer cell resistant to an FDA-approved therapy. In certain embodiments, the target cell is a cancer cell that is resistant to a macrophage-directed immunotherapy and/or targeted agent. In certain embodiments, the target cell is a cancer cell that is resistant to a macrophage-directed immunotherapy. In certain embodiments, the target cell is a cancer cell that is resistant to a targeted agent.

In some embodiments, the cancer comprises a solid tumor or hematological malignancy. In some embodiments, the cancer is or comprises a solid tumor. In some embodiments, the cancer is or comprises a hematological malignancy. In some embodiments, the cancer is a leukemia; a lymphoma; myelodysplasia; multiple myeloma; lung cancer (e.g., bronchogenic carcinoma, small cell lung cancer (SCLC), non-small cell lung cancer (NSCLC), adenocarcinoma of the lung); kidney cancer; acoustic neuroma; adenocarcinoma; adrenal gland cancer; anal cancer; angiosarcoma; appendix cancer; benign monoclonal gammopathy; biliary cancer; bladder cancer; breast cancer; brain cancer; bronchus cancer; carcinoid tumor; cervical cancer; choriocarcinoma; chordoma; craniopharyngioma; colorectal cancer; connective tissue cancer; epithelial carcinoma; ependymoma; endotheliosarcoma; endometrial cancer; esophageal cancer; Ewing's sarcoma; ocular cancer; familiar hypereosinophilia; gall bladder cancer; gastric cancer; gastrointestinal stromal tumor (GIST); germ cell cancer; head and neck cancer; heavy chain disease; leiomyosarcoma (LMS); mastocytosis; muscle cancer; myelodysplastic syndrome (MDS); mesothelioma; myeloproliferative disorder (MPD); neuroblastoma; neurofibroma; neuroendocrine cancer; osteosarcoma; ovarian cancer; papillary adenocarcinoma; pancreatic cancer; penile cancer; pinealoma; primitive neuroectodermal tumor (PNT); plasma cell neoplasia; paraneoplastic syndromes; intraepithelial neoplasms; prostate cancer; rectal cancer; rhabdomyosarcoma; salivary gland cancer; skin cancer; small bowel cancer; soft tissue sarcoma; sebaceous gland carcinoma; small intestine cancer; sweat gland carcinoma; synovioma; testicular cancer; thyroid cancer; urethral cancer; vaginal cancer; or vulvar cancer.

In certain embodiments, the cancer is bladder cancer, cervical cancer, dermatofibrosarcoma protuberans, endocrine tumors, neuroendocrine tumors, neuroblastoma, lung cancer (e.g., non-small cell lung cancer), anaplastic large cell lymphoma, glioblastoma multiforme, bile duct cancer, ovarian cancer, stomach cancer, colon cancer, rectal cancer, melanoma, colorectal cancer, brain cancer, head and neck cancer, thyroid cancer, soft tissue cancer, lung cancer, colon cancer, kidney cancer (e.g., papillary renal carcinoma), liver cancer, gastric cancer, gastrointestinal stromal tumor, giant cell tumor, esophageal cancer, gastroesophageal cancer, breast cancer, ovarian cancer, prostate cancer, endometrial cancer, pancreatic cancer, leukemia (e.g., acute myeloid leukemia), lymphoma, multiple myeloma, colon adenocarcinoma, lung adenocarcinoma, cutaneous melanoma, gastrointestinal cancer, anal cancer, glioblastoma, epithelian tumors of the head and neck, laryngeal cancer, oral cancer, myelodysplastic disorders, myeloproliferative disorders, ovarian epithelial cancer, fallopian tube cancer, primary peritoneal cancer, plexiform neurofibroma, skin cancer, soft tissue sarcoma, solid tumors with an NTRK gene fusion, or systemic mastocytosis.

In certain embodiments, the cancer is neuroblastoma, lung cancer (e.g., non-small cell lung cancer), anaplastic large cell lymphoma, glioblastoma multiforme, bile duct cancer, ovarian cancer, stomach cancer, colon cancer, rectal cancer, melanoma, colorectal cancer, brain cancer, head and neck cancer, thyroid cancer, soft tissue cancer, lung cancer, colon cancer, kidney cancer (e.g., papillary renal carcinoma), liver cancer, gastric cancer, gastroesophageal cancer, breast cancer, ovarian cancer, prostate cancer, endometrial carcinoma, pancreatic cancer, leukemia (e.g., acute myeloid leukemia), colon adenocarcinoma, lung adenocarcinoma, cutaneous melanoma, gastrointestinal cancer, anal cancer, glioblastoma, epithelian tumors of the head and neck, laryngeal cancer, and oral cancer. In certain embodiments, the cancer is lung cancer. In some embodiments, the cancer is non-small cell lung cancer (NSCLC).

In certain embodiments, the cancer is a cancer that is commonly treated with a targeted agent. In certain embodiments, the cancer is a cancer with a driver mutation that can be treated with a targeted agent directed at that driver mutation.

In certain embodiments, the cancer is associated with overexpressed and/or mutated ALK, such as neuroblastoma, non-small cell lung cancer, and anaplastic large cell lymphoma.

In certain embodiments, the cancer is associated with overexpressed and/or mutated ROS1, such as non-small cell lung cancer, glioblastoma multiforme, bile duct cancer, ovarian cancer, stomach cancer, colon cancer, and rectal cancer.

In certain embodiments, the cancer is associated with overexpressed and/or mutated BRAF, such as melanoma and colorectal cancer.

In certain embodiments, the cancer is associated with NTRK gene fusions, such as brain cancer, head and neck cancer, thyroid cancer, soft tissue cancer, lung cancer, and colon cancer.

In certain embodiments, the cancer is associated with overexpressed and/or mutated RET, such as non-small cell lung cancer and thyroid cancer.

In certain embodiments, the cancer is associated with overexpressed and/or mutated MET, such as kidney cancer (e.g., papillary renal carcinoma), liver cancer, and head and neck cancer.

In certain embodiments, the cancer is associated with overexpressed and/or mutated HER2, such as gastric/gastroesophageal cancer, breast cancer, and ovarian cancer.

In certain embodiments, the cancer is associated with overexpressed and/or mutated FGFR1, such as lung cancer, gastric cancer, prostate cancer, and breast cancer.

In certain embodiments, the cancer is associated with overexpressed and/or mutated FGFR2, such as gastric cancer, breast cancer, and endometrial carcinoma.

In certain embodiments, the cancer is associated with overexpressed and/or mutated KRAS, such as non-small cell lung cancer, colorectal cancer, and pancreatic cancer.

In certain embodiments, the cancer is associated with overexpressed and/or mutated FLT-3, such as acute myeloid leukemia, colon adenocarcinoma, lung adenocarcinoma, cutaneous melanoma, colorectal cancer, and breast cancer.

In certain embodiments, the cancer is associated with overexpressed and/or mutated C-Kit, such as gastrointestinal cancer, melanoma, thyroid carcinoma, and breast cancer.

In certain embodiments, the cancer is associated with overexpressed and/or mutated EGFR, such as non-small cell lung cancer, adenocarcinoma of the lung, anal cancer, glioblastoma, and epithelian tumors of the head and neck.

In certain embodiments, the cancer is associated with overexpressed and/or mutated SHP2, such as breast cancer, leukemia, lung cancer, liver cancer, gastric cancer, laryngeal cancer, and oral cancer.

In certain embodiments, the target cells are bladder cancer cells, cervical cancer cells, dermatofibrosarcoma protuberans cells, endocrine tumor cells, neuroendocrine tumor cells, neuroblastoma cells, lung cancer cells, glioblastoma multiforme cells, bile duct cancer cells, ovarian cancer cells, stomach cancer cells, colon cancer cells, rectal cancer cells, melanoma cells, colorectal cancer cells, brain cancer cells, head and neck cancer cells, thyroid cancer cells,, soft tissue cancer cells, kidney cancer cells, liver cancer cells, gastric cancer cells, gastrointestinal stromal tumor cells, giant cell tumor cells, esophageal cancer cells, gastroesophageal cancer cells, breast cancer cells, prostate cancer cells, endometrial cancer cells, pancreatic cancer cells, leukemia cells, lymphoma, cells multiple myeloma cells, colon adenocarcinoma cells, lung adenocarcinoma cells, cutaneous melanoma cells, gastrointestinal cancer cells, anal cancer cells, glioblastoma cells, laryngeal cancer cells, oral cancer cells, myelodysplastic disorder cells, myeloproliferative disorder cells, ovarian epithelial cancer cells, fallopian tube cancer cells, primary peritoneal cancer cells, plexiform neurofibroma cells, skin cancer cells, or soft tissue sarcoma cells

In certain embodiments, the target cell is a solid tumor cell, a breast cancer cell, a pancreatic cancer cell, a colon cancer cell, a non-small cell lung cancer cell, a leukemia cell, a lymphoma cell, or a myeloma cell. In certain embodiments, the target cell is a lung cancer cell or colon cancer cell. In certain embodiments, the target cell is a lung cancer cell. In certain embodiments, the target cell is a non-small cell lung cancer cell. In certain embodiments, the target cell is a colon cancer cell.

In certain embodiments, the population of target cells comprise at least 1,000, at least 1,500, at least 2,000, at least 2,500, at least 3,000, at least 3,500, at least 4,000, at least 4,500, at least 5,000, at least 5,500, at least 6,000, at least 6,500, at least 7,000, at least 7,500, at least 8,000, at least 8,500, at least 9,000, at least 9,500, at least 10,000 at least 11,000, at least 12,000, at least 13,000, at least 14,000, at least 15,000, at least 16,000, at least 17,000, at least 18,000, at least 19,000, or at least 20,000 cells. In certain embodiments, the population of macrophages comprises about 1,000, about 1,500, about 2,000, about 2,500, about 3,000, about 3,500, about 4,000, about 4,500, about 5,000, about 5,500, about 6,000, about 6,500, about 7,000, about 7,500, about 8,000, about 8,500, about 9,000, about 9,500, about 10,000 about 11,000, about 12,000, about 13,000, about 14,000, about 15,000, about 16,000, about 17,000, about 18,000, about 19,000, or about 20,000 cells. In certain embodiments, the population of target cells comprises about 1,000 to about 20,000 cells. In certain embodiments, the population of target cells comprises about 5,000 to about 20,000 cells. In certain embodiments, the population of target cells comprises at least 5,000 to about 20,000 cells. In certain embodiments, the population of target cells comprises about 5,000 to about 15,000 cells. In certain embodiments, the population of target cells comprises about 1,000 to about 5,000 cells. In certain embodiments, the population of target cells comprises about 2,000 to about 3,000 cells. In certain embodiments, the population of target cells comprises about 5,000 to about 10,000 cells. In certain embodiments, the population of target cells comprises about 2,000 to about 3,000 cells. In certain embodiments, the population of target cells comprises about 2,500 cells. In certain embodiments, the population of target cells comprises about 7,000 to about 8,000 cells. In certain embodiments, the population of target cells comprises about 7,500 cells.

### Determining Growth of Target Cell Populations

In certain embodiments, the target cell expresses a reporter protein such that the growth or destruction of the target cell may be monitored by detecting the presence or absence of the target cell. In certain embodiments, the target cell expresses a reporter protein that emits light. In certain embodiments, the target cell expresses green fluorescent protein. In certain embodiments, the target cell expresses red fluorescent protein.

In certain embodiments, the target cell comprises a label that emits light. In certain embodiments, the label is carboxyfluorescein succinimidyl ester. In certain embodiments, the label is pHrodo.

The use of reporter proteins that emit light allow the determination of growth and/or destruction of the populations of target cells by quantifying the light emission signal from the target cells. In certain embodiments, determining the amount of growth and/or destruction of the population of target cells at a timepoint comprises automated microscopy and/or image analysis to quantify the growth of the population of target cells. In certain embodiments, the automated microscopy and/or image analysis comprises detecting light emission from target cells expressing a reporter protein that emits light (e.g., green fluorescent protein or red fluorescent protein). In certain embodiments, the amount of growth and/or destruction of the target cell population is quantified by measuring the total area of the light-emitting reporter protein in the assay. In certain embodiments, the automated microscopy and/or image analysis comprises detecting light emission over the entirety of the assay mixture. In certain embodiments, the assay mixtures are each in one well of a 384-well plate. In certain embodiments, the assay mixtures are each in one well of a 384-well plate such that the assaying method may be achieved in high throughput fashion.

In certain embodiments, the amount of growth of target cell populations over time are determined by calculating a ratio of growth in the assay mixture versus the control mixture (i.e., no candidate agent present).

In certain embodiments, the timepoint for determining and comparing the amount of growth of the population of target cells with the control mixture is at least 4 hours, least 8 hours, least 12 hours, least 16 hours, least 20 hours, or least 24 hours after beginning culturing the assay mixture. In certain embodiments, the timepoint for determining and comparing the amount of growth of the population of target cells with the control mixture is at least 24 hours after beginning culturing the assay mixture.

In certain embodiments, the timepoint for determining and comparing the amount of growth of the population of target cells with the control mixture is at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, or at least 14 days after beginning culturing the assay mixture. In certain embodiments, the timepoint for determining and comparing the amount of growth of the population of target cells with the control mixture is at least 6 days after beginning culturing the assay mixture.

In certain embodiments, the timepoint for determining and comparing the amount of growth of the population of target cells with the control mixture is about 1 day, about 2 days, about 3 days, about 4 days, about 5 days, about 6 days, about 7 days, about 8 days, about 9 days, about 10 days, about 11 days, about 12 days, about 13 days, or about 14 days after beginning culturing the assay mixture. In certain embodiments, the timepoint for determining and comparing the amount of growth of the population of target cells with the control mixture is about 7 days after beginning culturing the assay mixture.

In certain embodimentsm the timepoint for determining and comparing the amount of growth of the population of target cells with the control mixture is immediately after completing culturing the assay mixture.

### Culturing

In certain embodiments, culturing the assay mixture takes at least 4, at least 8, at least 12, at least 16, at least 20, or at least 24 hours. In certain embodiments, culturing the assay mixture takes at least 24 hours. In certain embodiments, culturing the assay mixture takes at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, or at least 14 days. In certain embodiments, culturing the assay mixture takes about 1 day, about 2 days, about 3 days, about 4 days, about 5 days, about 6 days, about 7 days, about 8 days, about 9 days, about 10 days, about 11 days, about 12 days, about 13 days, or about 14 days.

In certain embodiments, culturing the assay mixture takes place at room temperature. In certain embodiments, culturing the assay mixture takes place at a temperature above room temperature. In certain embodiments, culturing the assay mixture takes place at a temperature of at least 20° C., at least 25° C., at least 30° C., at least 35° C., at least 40° C., at least 45° C., or at least 50° C. In certain embodiments, culturing the assay mixture takes place at a temperature of about 20° C. to about 40° C. In certain embodiments, culturing the assay mixture takes place at a temperature of about 30° C. to about 40° C. In certain embodiments. culturing the assay mixture takes place at a temperature of about 25° C. to about 30° C. In certain embodiments, culturing the assay mixture takes place at a temperature of about 35° C. to about 40° C. In certain embodiments, culturing the assay mixture takes place at physiological temperature. In certain embodiments, culturing the assay mixture takes place at about 36° C. In certain embodiments, culturing the assay mixture takes place at about 37° C.

### Agents

In certain embodiments, the agent is a small molecule. In certain embodiments, the targeted agent is a biologic. In certain embodiments, the agent is a polypeptide, a cytokine, or an antibody or fragment thereof.

In certain embodiments, the agent is a targeted agent. In certain embodiments, the agent is a tyrosine kinase inhibitor. In certain embodiments, the agent is a VEGF inhibitor, an ALK inhibitor, a ROS1 inhibitor, a BRAF inhibitor, a MEK inhibitor, a NTRK inhibitor, a RET inhibitor, a MET inhibitor, a HER2 inhibitor, an FGFR1 inhibitor, an FGFR2 inhibitor, a KRAS inhibitor, a FLT-3 inhibitor, a C-Kit inhibitor, an EGFR inhibitor, or an SHP2 inhibitor. In certain embodiments, the agent is an ALK inhibitor, a MEK inhibitor, a KRAS inhibitor, an EGFR inhibitor, or an SHP2 inhibitor.

In certain embodiments, the agent is an ALK inhibitor, a KRAS inhibitor, or an EGFR inhibitor. In certain embodiments, the agent is an ALK inhibitor. In certain embodiments, the agent is a KRAS inhibitor. In certain embodiments, the agent is an EGFR inhibitor.

In certain embodiments, the candidate agent is a member of a library of candidate agents. In certain embodiments, the candidate agent is a member of a library of candidate agents such that the assaying method is achieved in a high throughput fashion. In certain embodiments, the assaying method is achieved by performing in a 384-well plate. In certain embodiments, the candidate agent is added multiple times throughout the course of the assay. For example, a candidate agent may degrade over time. In certain embodiments, the candidate agent is added once daily over the course of the assay. In certain embodiments, the candidate agent is added every other day over the course of the assay. In certain embodiments, the candidate agent is added once, twice, three times, four times, five times, six times, seven times, eight times, nine times, or ten times over the course of the assay (e.g during the culturing step).

In certain embodiments, the agent is an agent that modulates macrophage function when the amount of growth of the population of target cells in the assay mixture is different from the amount of growth of the population of target cells in the control mixture at the same timepoint. In certain embodiments, the difference is at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 80%, at least 95%, or at least 100%.

In certain embodiments, the agent is an activator of macrophage function such that the amount of growth of the population of target cells in the assay mixture is less than the amount of growth of the population of target cells in the control mixture. In certain embodiments, the amount of growth is at least 1% lower, at least 2% lower, at least 3% lower, at least 4% lower, at least 5% lower, at least 6% lower, at least 7% lower, at least 8% lower, at least 9% lower, at least 10% lower, at least 15% lower, at least 20% lower, at least 25% lower, at least 30% lower, at least 35% lower, at least 40% lower, at least 45% lower, at least 50% lower, at least 55% lower, at least 60% lower, at least 65% lower, at least 70% lower, at least 75% lower, at least 80% lower, at least 85% lower, at least 80% lower, at least 95% lower, or at least 100% lower.

In certain embodiments, the population of target cells in the assay mixture at the timepoint for determining the amount of growth of the population of target cells is less than the population of target cells in the assay mixture at the time of adding the candidate agent to the mixture. In certain embodiments, the population is at least 1% lower, at least 2% lower, at least 3% lower, at least 4% lower, at least 5% lower, at least 6% lower, at least 7% lower, at least 8% lower, at least 9% lower, at least 10% lower, at least 15% lower, at least 20% lower, at least 25% lower, at least 30% lower, at least 35% lower, at least 40% lower, at least 45% lower, at least 50% lower, at least 55% lower, at least 60% lower, at least 65% lower, at least 70% lower, at least 75% lower, at least 80% lower, at least 85% lower, at least 80% lower, at least 95% lower, or at least 100% lower.

In certain embodiments, the agent is determined to be an agent that promotes macrophage-mediated cytotoxicity when the amount of growth of the population of target cells in the assay mixture is less than the amount of growth of the population of target cells in the control mixture at the same timepoint. In certain embodiments, the amount of growth is at least 1% lower, at least 2% lower, at least 3% lower, at least 4% lower, at least 5% lower, at least 6% lower, at least 7% lower, at least 8% lower, at least 9% lower, at least 10% lower, at least 15% lower, at least 20% lower, at least 25% lower, at least 30% lower, at least 35% lower, at least 40% lower, at least 45% lower, at least 50% lower, at least 55% lower, at least 60% lower, at least 65% lower, at least 70% lower, at least 75% lower, at least 80% lower, at least 85% lower, at least 80% lower, at least 95% lower, or at least 100% lower.

In certain embodiments, the agent is determined to be an agent that promotes macrophage anti-cancer function when the amount of growth of the population of target cancer cells in the assay mixture is lower than the amount of growth of the population of target cancer cells in the control mixture at the same timepoint. In certain embodiments, the amount of growth is at least 1% lower, at least 2% lower, at least 3% lower, at least 4% lower, at least 5% lower, at least 6% lower, at least 7% lower, at least 8% lower, at least 9% lower, at least 10% lower, at least 15% lower, at least 20% lower, at least 25% lower, at least 30% lower, at least 35% lower, at least 40% lower, at least 45% lower, at least 50% lower, at least 55% lower, at least 60% lower, at least 65% lower, at least 70% lower, at least 75% lower, at least 80% lower, at least 85% lower, at least 80% lower, at least 95% lower, or at least 100% lower.

In certain embodiments, the agent is determined to be a cancer therapy when the amount of growth of the population of target cancer cells in the assay mixture is lower than the amount of growth of the population of target cancer cells in the control mixture at the same timepoint. In certain embodiments, the amount of growth is at least 1% lower, at least 2% lower, at least 3% lower, at least 4% lower, at least 5% lower, at least 6% lower, at least 7% lower, at least 8% lower, at least 9% lower, at least 10% lower, at least 15% lower, at least 20% lower, at least 25% lower, at least 30% lower, at least 35% lower, at least 40% lower, at least 45% lower, at least 50% lower, at least 55% lower, at least 60% lower, at least 65% lower, at least 70% lower, at least 75% lower, at least 80% lower, at least 85% lower, at least 80% lower, at least 95% lower, or at least 100% lower.

In certain embodiments, the agent that modulates macrophage function is an inhibitor of macrophage function such that the amount of growth of the population of target cells in the assay mixture is greater than the amount of growth of the population of target cells in the control mixture. In certain embodiments, the amount of growth is at least 1% higher, at least 2% higher, at least 3% higher, at least 4% higher, at least 5% higher, at least 6% higher, at least 7% higher, at least 8% higher, at least 9% higher, at least 10% higher, at least 15% higher, at least 20% higher, at least 25% higher, at least 30% higher, at least 35% higher, at least 40% higher, at least 45% higher, at least 50% higher, at least 55% higher, at least 60% higher, at least 65% higher, at least 70% higher, at least 75% higher, at least 80% higher, at least 85% higher, at least 80% higher, at least 95% higher, or at least 100% higher.

In certain embodiments, the population of target cells in the assay mixture at the timepoint for determining the amount of growth of the population of target cells is greater than the population of target cells in the assay mixture at the time of adding the candidate agent to the mixture. In certain embodiments, the amount of growth is at least 1% higher, at least 2% higher, at least 3% higher, at least 4% higher, at least 5% higher, at least 6% higher, at least 7% higher, at least 8% higher, at least 9% higher, at least 10% higher, at least 15% higher, at least 20% higher, at least 25% higher, at least 30% higher, at least 35% higher, at least 40% higher, at least 45% higher, at least 50% higher, at least 55% higher, at least 60% higher, at least 65% higher, at least 70% higher, at least 75% higher, at least 80% higher, at least 85% higher, at least 80% higher, at least 95% higher, or at least 100% higher.

### Pharmaceutical Compositions

The present disclosure also provides pharmaceutical compositions comprising an agent identified by a method described herein, or a pharmaceutically acceptable salt, solvate, hydrate, polymorph, co-crystal, tautomer, stereoisomer, or prodrug thereof, and optionally a pharmaceutically acceptable excipient. In certain embodiments, the agent described herein is provided in an effective amount in the pharmaceutical composition. In certain embodiments, the effective amount is a therapeutically effective amount. In certain embodiments, the effective amount is a prophylactically effective amount, infectious diseases, cardiovascular disease, metabolic disorders, or cancer

In certain embodiments, the effective amount of an agent is an amount effective for treating a metabolic disorder in a subject in need thereof. In certain embodiments, the effective amount of an agent is an amount effective for treating an infectious disease in a subject in need thereof. In certain embodiments, the effective amount of an agent is an amount effective for treating a cardiovascular disease in a subject in need thereof. In certain embodiments, the effective amount of an agent is an amount effective for treating cancer in a subject in need thereof. In certain embodiments, the effective amount of an agent is an amount effective for activating macrophage function in a subject or biological sample. In certain embodiments, the effective amount of an agent is an amount effective for inhibiting macrophage function in a subject or biological sample.

The pharmaceutical compositions described herein may be useful in treating and/or preventing cancer in a subject in need thereof, such as cancers that are resistant to or are at risk of becoming resistant to a targeted agent and/or a macrophage-directed immunotherapy.

In certain embodiments, the subject is an animal. The animal may be of either sex and may be at any stage of development. In certain embodiments, the subject described herein is a human. In certain embodiments, the subject is a non-human animal. In certain embodiments, the subject is a mammal. In certain embodiments, the subject is a non-human mammal. In certain embodiments, the subject is a domesticated animal, such as a dog, cat, cow, pig, horse, sheep, or goat. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal, such as a rodent (e.g., mouse, rat), dog, pig, or non-human primate. In certain embodiments, the animal is a genetically engineered animal. In certain embodiments, the animal is a transgenic animal (e.g., transgenic mice and transgenic pigs). In certain embodiments, the subject is a fish or reptile. In certain embodiments, the subject is with a cancer. In certain embodiments, the subject is with a cancer and has failed therapy of the cancer with an FDA approved therapy (e.g., EGFR inhibitor) alone. In certain embodiments, the subject is with a cancer and has failed therapy of the cancer with a macrophage-directed immunotherapy alone.

Pharmaceutical compositions described herein can be prepared by any method known in the art of pharmacology. In general, such preparatory methods include bringing the macrophage-directed immunotherapy and/or targeted agents described herein (i.e., the “active ingredients”) into association with a carrier or excipient, and/or one or more other accessory ingredients, and then, if necessary and/or desirable, shaping, and/or packaging the product into a desired single-or multi-dose unit.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as one-half or one-third of such a dosage.

Relative amounts of the active ingredient, the pharmaceutically acceptable excipient, and/or any additional ingredients in a pharmaceutical composition described herein will vary, depending upon the identity, size, and/or condition of the subject treated and further depending upon the route by which the composition is to be administered. The composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate. quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan (Tween® 60), polyoxyethylene sorbitan monooleate (Tween® 80). sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutol®), sucrose fatty acid esters, polyethylene glycol fatty acid esters (e.g., Cremophor®), polyoxyethylene ethers, (e.g., polyoxyethylene lauryl ether (Brij® 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, poloxamer P-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone). magnesium aluminum silicate (Veegum®), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben. Germall® 115, Germaben® II, Ncolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates described herein are mixed with solubilizing agents such as Cremophor®, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution. U.S.P., and isotonic sodium chloride solution. In addition, sterile, fixed oils are conventionally employed as a solvent or suspending medium. For this purpose any bland fixed oil can be employed including synthetic mono-or di-glycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form may be accomplished by dissolving or suspending the drug in an oil vehicle.

Compositions for rectal or vaginal administration are typically suppositories which can be prepared by mixing the conjugates described herein with suitable non-irritating excipients or carriers such as cocoa butter, polyethylene glycol, or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose. alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the art of pharmacology. They may optionally comprise opacifying agents and can be of a composition that they release the active ingredient(s) only, or preferentially, in a certain part of the intestinal tract, optionally, in a delayed manner. Examples of encapsulating compositions which can be used include polymeric substances and waxes. Solid compositions of a similar type can be employed as fillers in soft and hard-filled gelatin capsules using such excipients as lactose or milk sugar as well as high molecular weight polethylene glycols and the like.

The active ingredient can be in a micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of encapsulating agents which can be used include polymeric substances and waxes.

Dosage forms for topical and/or transdermal administration of a macrophage-directed immunotherapy and/or targeted agent described herein may include ointments. pastes, creams, lotions, gels, powders, solutions, sprays, inhalants, and/or patches. Generally, the active ingredient is admixed under sterile conditions with a pharmaceutically acceptable carrier or excipient and/or any needed preservatives and/or buffers as can be required. Additionally, the present disclosure contemplates the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Suitable devices for use in delivering intradermal pharmaceutical compositions described herein include short needle devices. Intradermal compositions can be administered by devices which limit the effective penetration length of a needle into the skin. Alternatively or additionally, conventional syringes can be used in the classical mantoux method of intradermal administration. Jet injection devices which deliver liquid formulations to the dermis via a liquid jet injector and/or via a needle which pierces the stratum corneum and produces a jet which reaches the dermis are suitable. Ballistic powder/particle delivery devices which use compressed gas to accelerate the macrophage-directed immunotherapy and/or targeted agent in powder form through the outer layers of the skin to the dermis are suitable.

Formulations suitable for topical administration include, but are not limited to, liquid and/or semi-liquid preparations such as liniments, lotions, oil-in-water and/or water-in-oil emulsions such as creams, ointments, and/or pastes, and/or solutions and/or suspensions. Topically administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described herein.

A pharmaceutical composition described herein can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers, or from about 1 to about 6nanometers. Such compositions are conveniently in the form of dry powders for administration using a device comprising a dry powder reservoir to which a stream of propellant can be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65° F. at atmospheric pressure. Generally, the propellant may constitute 50 to 99.9% (w/w) of the composition, and the active ingredient may constitute 0.1 to 20% (w/w) of the composition. The propellant may further comprise additional ingredients such as a liquid non-ionic and/or solid anionic surfactant and/or a solid diluent (which may have a particle size of the same order as particles comprising the active ingredient).

Pharmaceutical compositions described herein formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface-active agent, and/or a preservative such as methylhydroxybenzoate. The droplets provided by this route of administration may have an average diameter in the range from about 0.1 to about 200 nanometers.

Formulations described herein as being useful for pulmonary delivery are useful for intranasal delivery of a pharmaceutical composition described herein. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares.

Formulations for nasal administration may, for example, comprise from about as little as 0.1% (w/w) to as much as 100% (w/w) of the active ingredient, and may comprise one or more of the additional ingredients described herein. A pharmaceutical composition described herein can be prepared, packaged, and/or sold in a formulation for buccal administration. Such formulations may, for example, be in the form of tablets and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

A pharmaceutical composition described herein can be prepared, packaged, and/or sold in a formulation for ophthalmic administration. Such formulations may, for example, be in the form of eye drops including, for example, a 0.1-1.0% (w/w) solution and/or suspension of the active ingredient in an aqueous or oily liquid carrier or excipient. Such drops may further comprise buffering agents, salts, and/or one or more other of the additional ingredients described herein. Other opthalmically-administrable formulations which are useful include those which comprise the active ingredient in microcrystalline form and/or in a liposomal preparation. Ear drops and/or eye drops are also contemplated as being within the scope of this disclosure.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

The agents provided herein are typically formulated in dosage unit form for ease of administration and uniformity of dosage. It will be understood, however, that the total daily usage of the compositions described herein will be decided by a physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

The agents and compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration). In certain embodiments, the macrophage-directed immunotherapies, targeted agents, and pharmaceutical compositions described herein are suitable for topical administration to the eye of a subject.

The exact amount (e.g., combined amount) of the agent(s) in the composition required to achieve an effective amount will vary from subject to subject, depending, for example, on species, age, and general condition of a subject, severity of the side effects or disorder, identity of the agent, mode of administration, and the like. An effective amount may be included in a single dose (e.g., single oral dose) or multiple doses (e.g., multiple oral doses). In certain embodiments, when multiple doses are administered to a subject or applied to a biological sample, tissue, or cell, the frequency of administering the multiple doses to the subject or applying the multiple doses to the biological sample, tissue, or cell is about three doses a day, about two doses a day, about one dose a day, about one dose every other day, about one dose every third day, about one dose every week, about one dose every about two weeks, about one dose every about three weeks, or about one dose every about four weeks. In certain embodiments, the frequency of administering the multiple doses to the subject or applying the multiple doses to the biological sample, tissue, or cell is about one dose per day. In certain embodiments, the frequency of administering the multiple doses to the subject or applying the multiple doses to the biological sample, tissue, or cell is about two doses per day. In certain embodiments, the frequency of administering the multiple doses to the subject or applying the multiple doses to the biological sample, tissue, or cell is about three doses per day. In certain embodiments, when multiple doses are administered to a subject or applied to a biological sample, tissue, or cell, the duration between the first dose and last dose of the multiple doses is about one day, about two days, about four days, about one week, about two weeks, about three weeks, about one month, about two months, about three months, about four months, about six months, about nine months, about one year, about two years, about three years, about four years, about five years, about seven years, about ten years, fifteen years, twenty years, or the lifetime of the subject, tissue, or cell. In certain embodiments, the duration between the first dose and last dose of the multiple doses is about three months, about six months, or about one year. In certain embodiments, the duration between the first dose and last dose of the multiple doses is the lifetime of the subject, tissue, or cell. In certain embodiments, a dose (e.g., a single dose, or any dose of multiple doses) described herein includes independently between 0.1 μg and 1 μg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 1 mg and 100 mg, between 30 mg and 100 mg, between 100 mg and 300 mg, between 1 mg and 1 g, between 300 mg and 1 g, between 1 mg and 10 g, or between 1 g and 10 g, inclusive, as the weight of an agent described herein.

Doses and dose ranges described herein provide guidance for the administration of provided pharmaceutical compositions to an adult (e.g., an adult whose body weight is 70 kg). The amount to be administered to, for example, a child or an adolescent can be determined by a medical practitioner or person skilled in the art and can be lower or the same as that administered to an adult.

An agent or composition, as described herein, can be administered in combination with one or more additional pharmaceutical agents (e.g., therapeutically and/or prophylactically active agents). The agent, or composition can be administered in combination with additional pharmaceutical agents that improve its activity (e.g., activity (e.g., potency and/or efficacy) in treating a cancer in a subject in need thereof, in preventing a cancer in a subject in need thereof, in reducing, delaying, and/or preventing in a subject in need thereof the resistance of cancers to a macrophage-directed immunotherapy and/or targeted agent, in inhibiting the proliferation of a cell, in reducing, delaying, and/or preventing the resistance of a cell to a macrophage-directed immunotherapy and/or targeted agent), improve bioavailability, improve safety, reduce drug resistance, reduce and/or modify metabolism, inhibit excretion, and/or modify distribution in a subject, biological sample, tissue, or cell. It will also be appreciated that the therapy employed may achieve a desired effect for the same disorder, and/or it may achieve different effects. In certain embodiments, a pharmaceutical composition described herein including (1) an agent described herein, and (2) an additional pharmaceutical agent shows a synergistic effect, compared with a pharmaceutical composition including one of (1) and (2), but not both (1) and (2).

The agent, or composition can be independently administered concurrently with, prior to, or subsequent to one or more additional pharmaceutical agents. In certain embodiments, the additional pharmaceutical agents and the macrophage-directed immunotherapy are not the same, and the additional pharmaceutical agents and the targeted agent are not the same. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease (e.g., proliferative disease). Each additional pharmaceutical agent may be administered at a dose and/or on a time schedule determined for that pharmaceutical agent. The additional pharmaceutical agents may also be administered together with each other and/or with the macrophage-directed immunotherapy, targeted agent, or composition described herein at the same time or administered separately at different times. The particular combination to employ in a regimen will take into account compatibility of the macrophage-directed immunotherapy and/or targeted agent described herein with the additional pharmaceutical agent(s), and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

The additional pharmaceutical agents include, but are not limited to, anti-proliferative agents, anti-cancer agents, anti-angiogenesis agents, anti-inflammatory agents, immunosuppressants, pain-relieving agents, and a combination thereof. In certain embodiments, the additional pharmaceutical agent is an anti-proliferative agent (e.g., anti-cancer agent, cytotoxic agent).

Also encompassed by the disclosure are kits (e.g., pharmaceutical packs). The kits provided may comprise an agent described herein, or a pharmaceutical composition described herein. The kits may comprise an agent in a first container. The kits may comprise a pharmaceutical composition in a first container. In some embodiments, the kits further include a second container comprising a pharmaceutical excipient for dilution or suspension of the agent, and/or pharmaceutical composition. Each of the first container and second container may independently be a vial, ampule, bottle, syringe, and/or dispenser package, or other suitable container. In certain embodiments, the kits are useful for treating a cancer (e.g., cancer that is resistant to a macrophage-directed immunotherapy and/or targeted agent) in a subject in need thereof. In certain embodiments, the kits are useful for preventing a cancer (e.g., cancer that is resistant to a macrophage-directed immunotherapy and/or targeted agent) in a subject in need thereof. A kit described herein may also include information as required by a regulatory agency such as the U.S. Food and Drug Administration (FDA). In certain embodiments, the information included in the kits is prescribing information. A kit described herein may include one or more additional pharmaceutical agents described herein as a separate composition.

### EXAMPLES

In order that the present disclosure may be more fully understood, the following examples are set forth. The examples described in this application are offered to illustrate the methods, agents, and compositions provided herein and are not to be construed in any way as limiting their scope.

### Unbiased Drug Screens Identify EGFR Inhibitors as Drugs That Sensitize Cancer Cells to Macrophage-Mediated Cytotoxicity

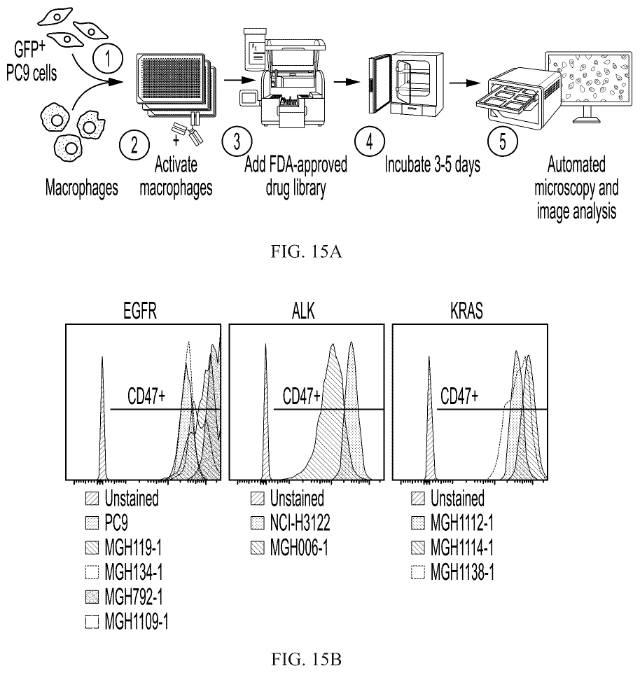

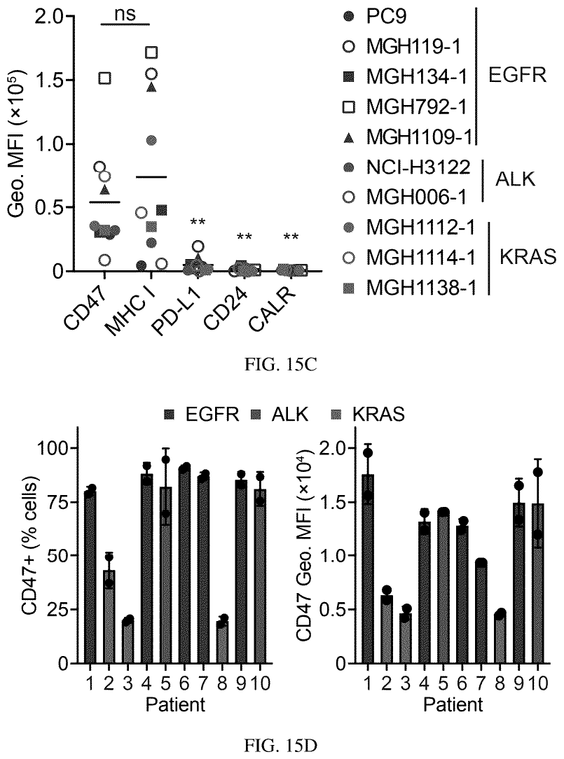

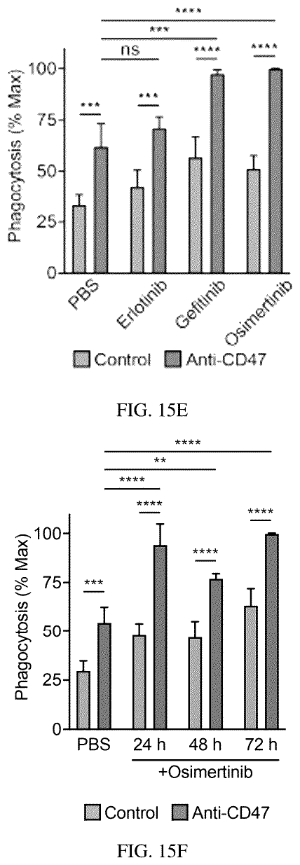

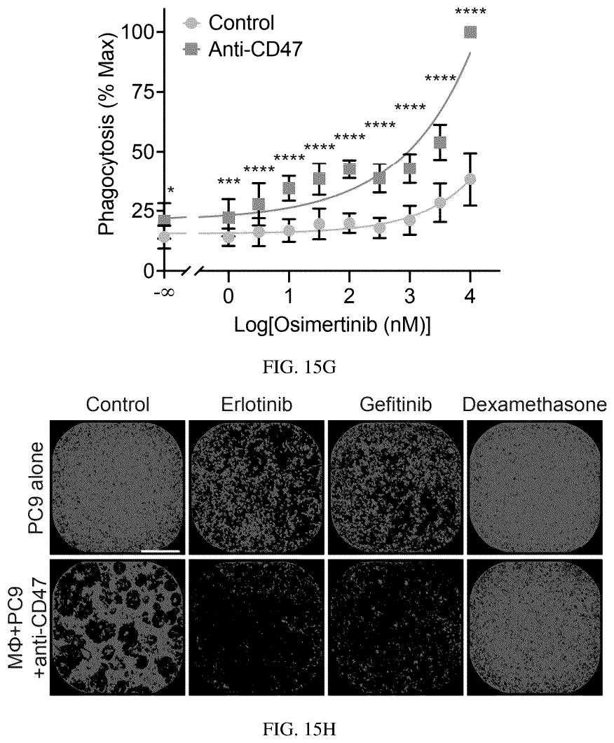

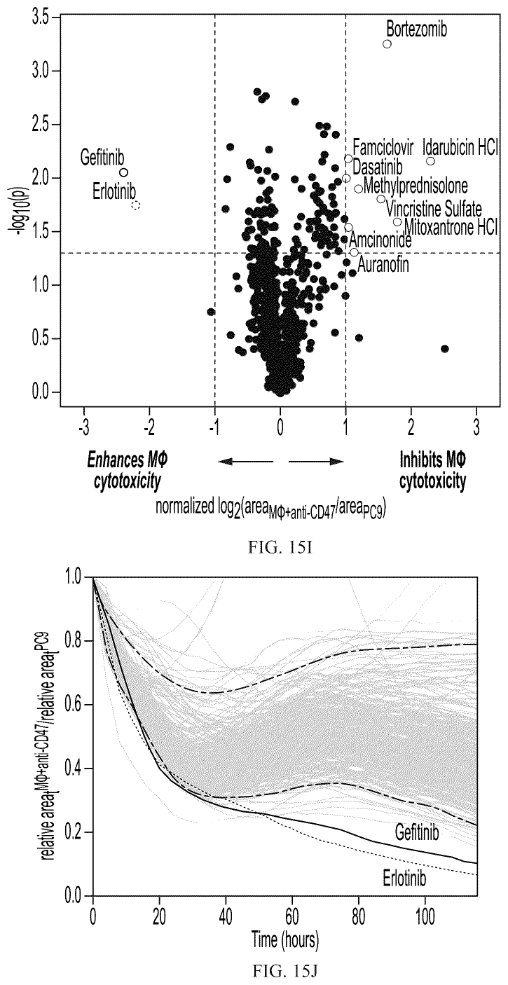

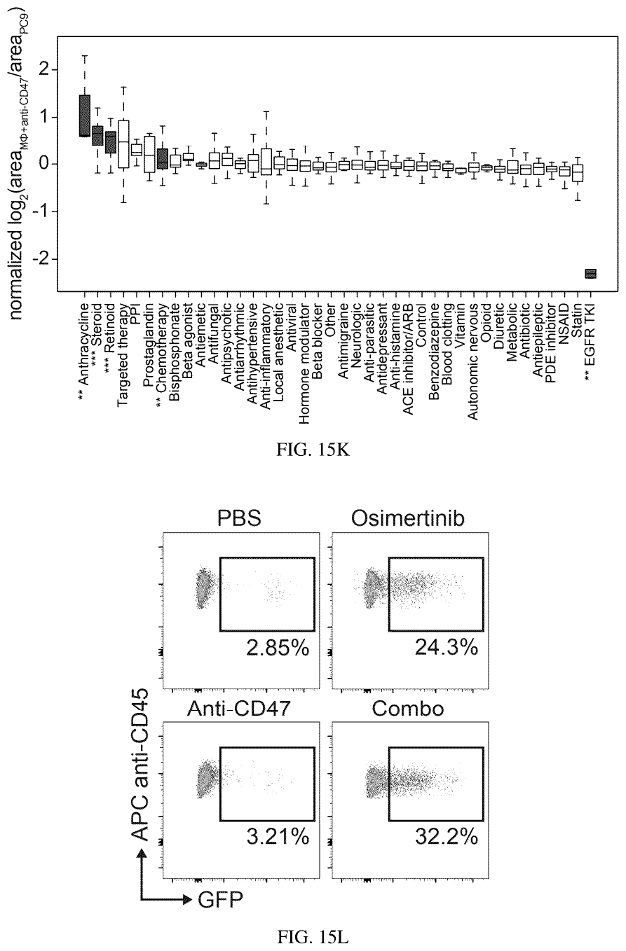

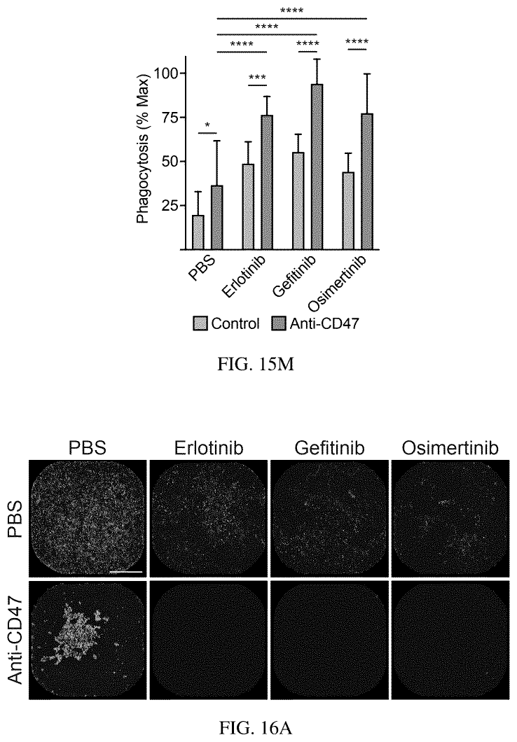

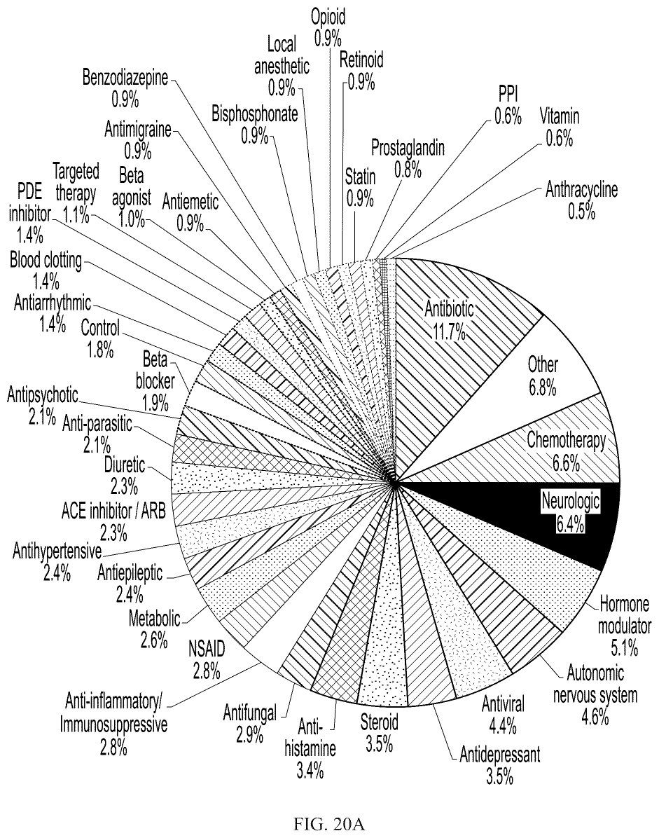

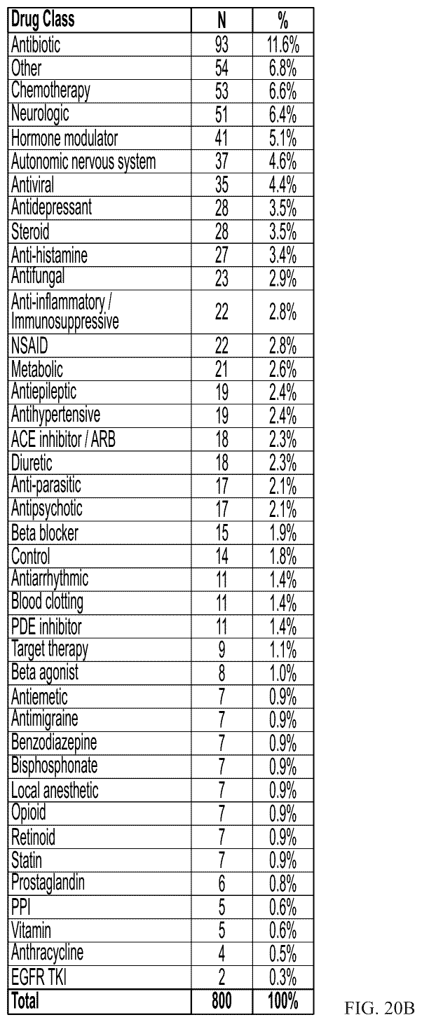

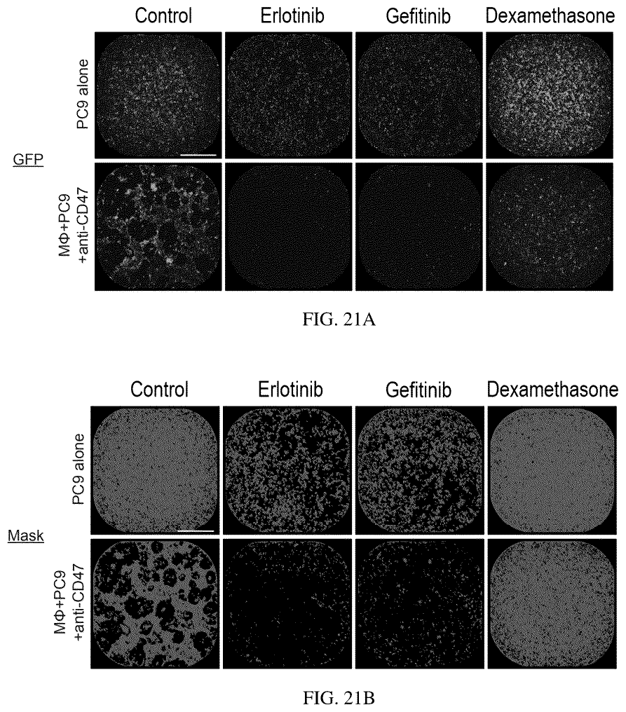

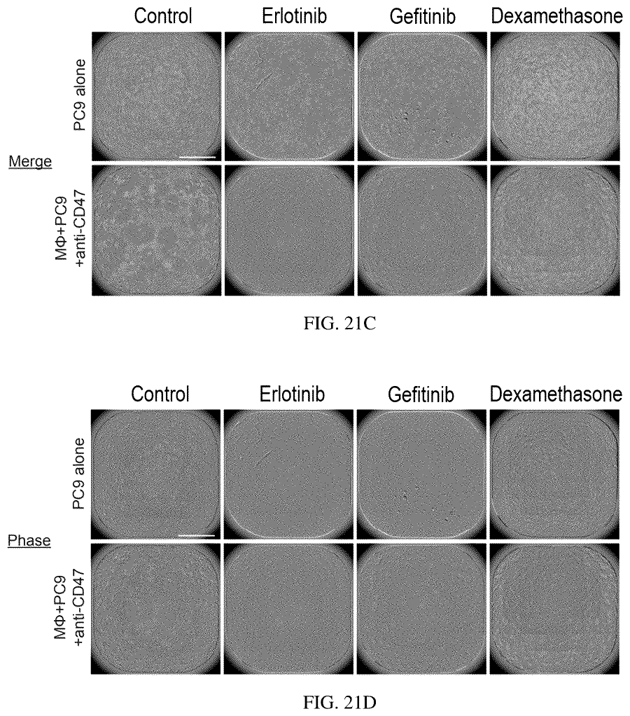

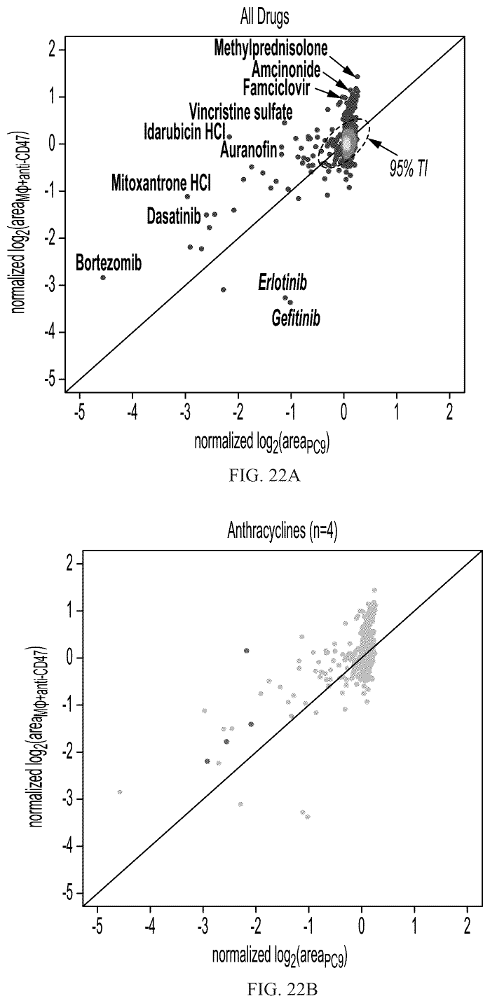

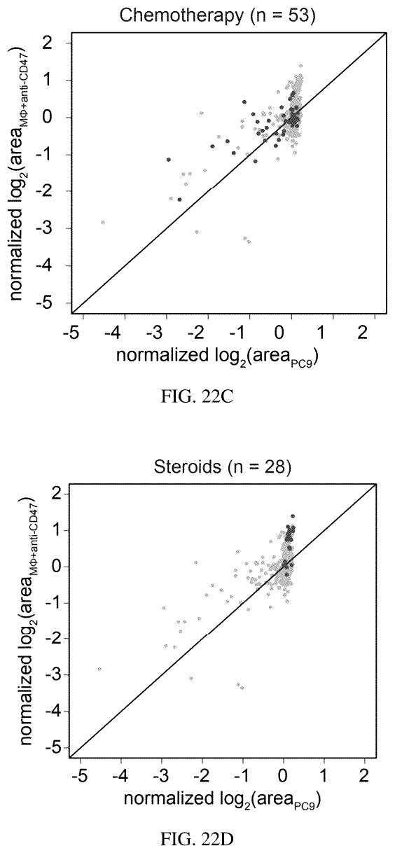

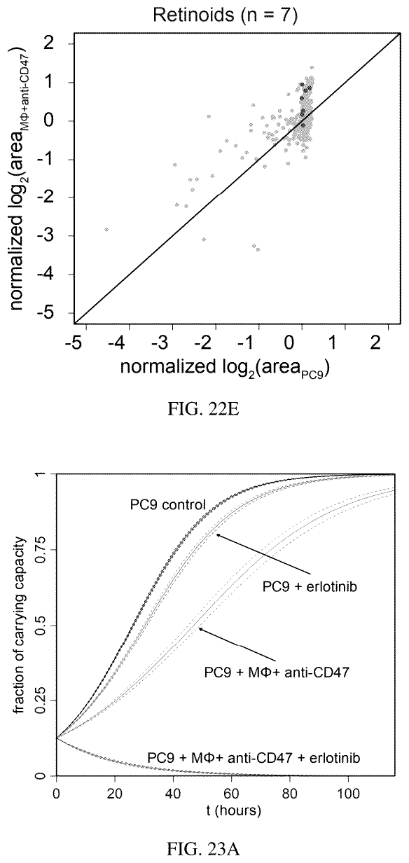

To identify drugs that make lung cancer cells more vulnerable to macrophage-mediated destruction, a novel, unbiased screening platform that measures macrophage anti-tumor function in a high-throughput manner was developed (FIG. 15A). We first employed this platform to study EGFR mutant lung cancer. We differentiated primary human macrophages ex vivo and co-cultured them in 384-well plates with GFP+ PC9 cells, a human EGFR mutant lung cancer cell line. A small molecule library of 800 FDA-approved drugs (FIGS. 20A-B) was added to the wells at 5.0 uM concentration along with a CD47-blocking antibody. We then cocultured the cells for 3-5 days and performed whole-well imaging to quantify the surviving GFP+ area. We evaluated the ability of each drug to kill the GFP+ PC9 cells in the presence of activated macrophages compared to GFP+ PC9 cells alone. From this analysis, we identified two drug classes that specifically inhibit macrophage anti-tumor function (steroids, retinoids), and two drug classes that are less effective when macrophages are present (anthracyclines, other chemotherapy drugs) (FIGS. 15H-15K, 21A-D, and 22A-E). In contrast, two EGFR tyrosine kinase inhibitors (TKIs)-erlotinib and gefitinib-markedly and specifically enhanced the ability of the macrophages to kill the PC9 cells. These drugs synergized with anti-CD47 therapy and resulted in over 4-fold enhancement of macrophage-mediated cytotoxicity (FIGS. 15H-15K, 21A-D, 22A-E, and 23A-B). Given that the PC9 cells contain an activating mutation in EGFR, we hypothesized that EGFR inhibitors act on the cancer cells to prime them for macrophage mediated-destruction.

### EGFR Inhibitors Promote Macrophage Phagocytosis of EGFR Mutant Lung Cancer Cells

To investigate the therapeutic potential of combining EGFR inhibitors with anti-CD47 antibodies, we first examined whether CD47 could be a genuine target for lung cancers bearing driver mutations. Using flow cytometry, we evaluated cell-surface expression of CD47 on established and patient-derived cell lines containing EGFR or KRAS driver mutations or oncogenic ALK fusions. We found that CD47 was highly expressed on the cell surface of all specimens tested (FIG. 15B). We also compared CD47 expression relative to other surface antigens that regulate macrophage activity, including MHC class I, PD-L1, CD24, and calreticulin. We found that both CD47 and MHC class I were highly expressed, whereas other macrophage immune checkpoint molecules were low or absent (FIG. 15C). We also examined CD47 expression on primary lung cancer cells from malignant pleural effusions and observed high CD47 expression on the cell surface (FIG. 15D).

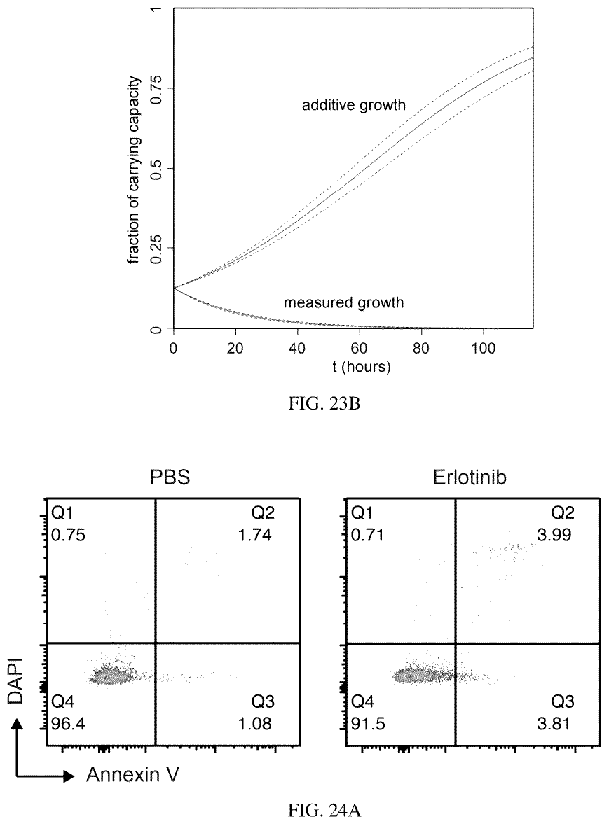

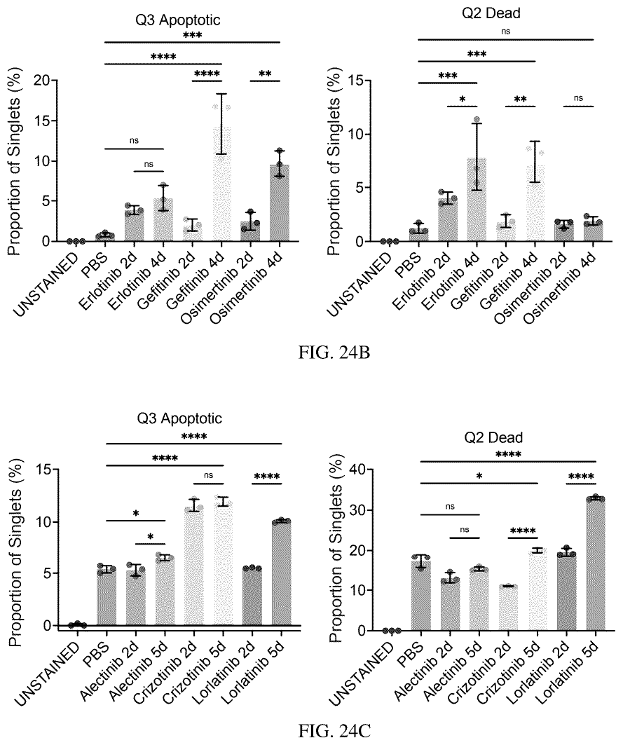

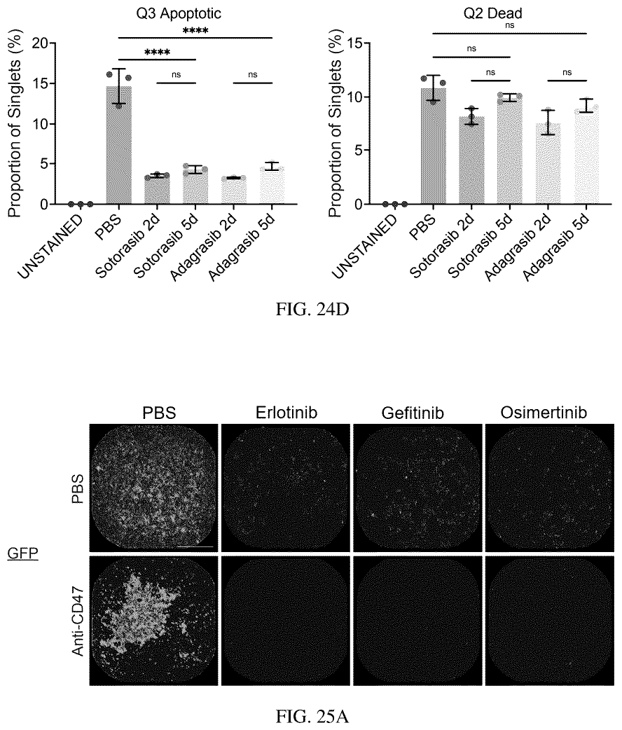

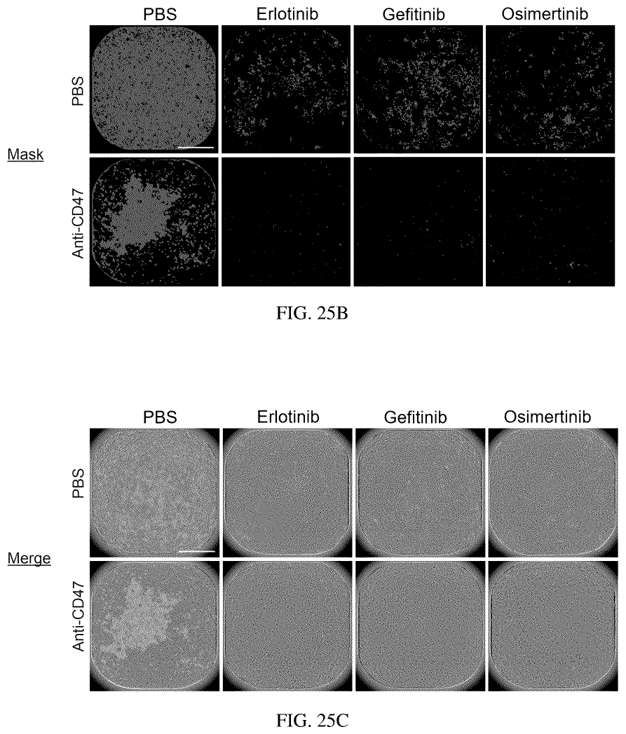

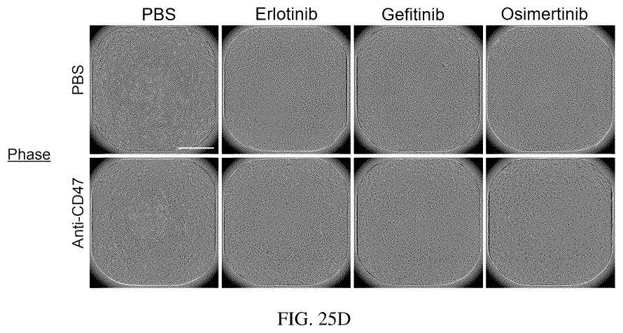

We next determined if CD47 could exert a functional role to protect lung cancer cells from macrophage phagocytosis, and whether treatment with EGFR TKIs could enhance phagocytosis. We exposed GFP+ PC9 cells to 1.0 uM TKI (erlotinib, gefitinib, or osimertinib) for 48 hours and then co-cultured the cells with primary human macrophages for 2 hours alone or with a CD47-blocking antibody. Regardless of which TKI was used, maximal phagocytosis occurred with the combination of an anti-CD47 antibody and TKI-treated cells (FIGS. 15L-15M). This effect was maximal within 24 hours of TKI exposure (FIG. 15F), a timepoint at which the cancer cells are actively proliferating and only exhibit minimal apoptosis (FIGS. 24A-D). Furthermore, this effect exhibited a dose-response relationship such that greater concentrations of osimertinib resulted in greater macrophage phagocytosis upon anti-CD47 treatment (FIG. 15G).

### The Combination of EGFR Inhibitors and Anti-CD47 Antibodies Eliminates Persister Cells In Vitro

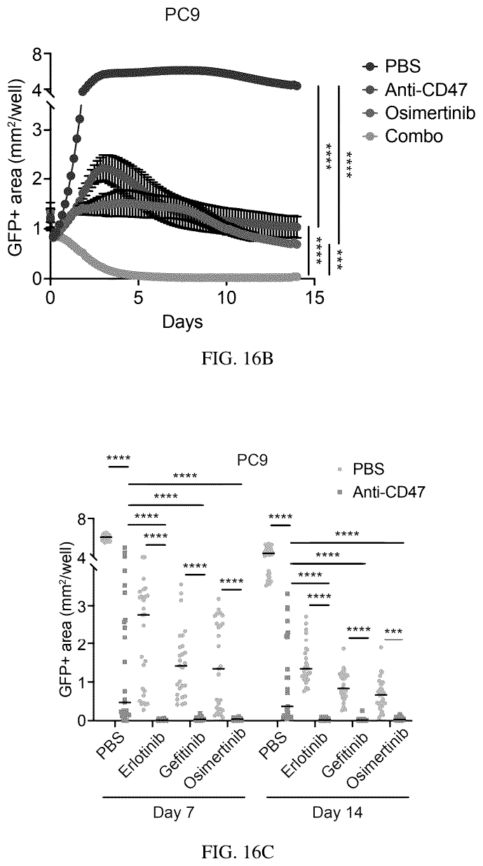

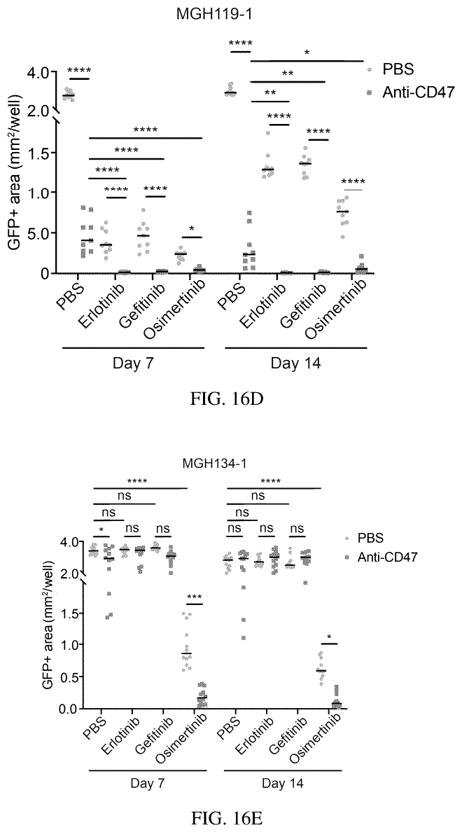

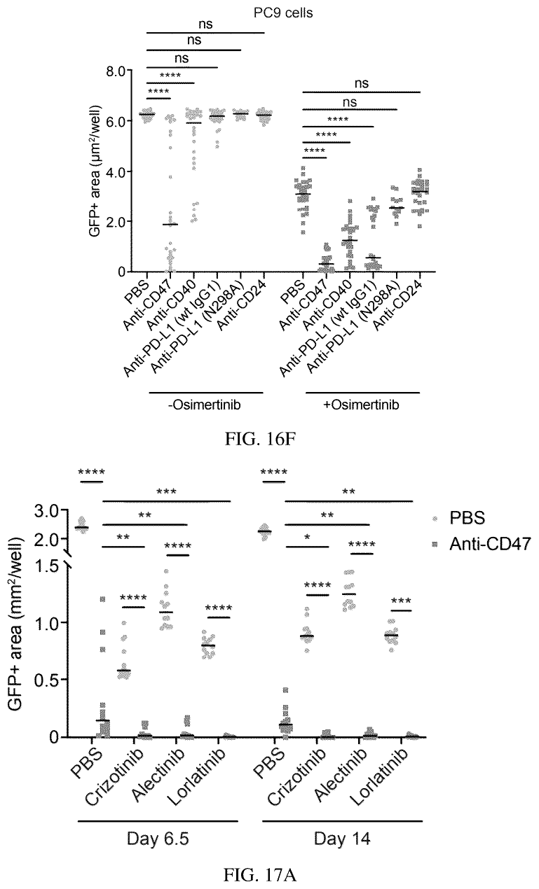

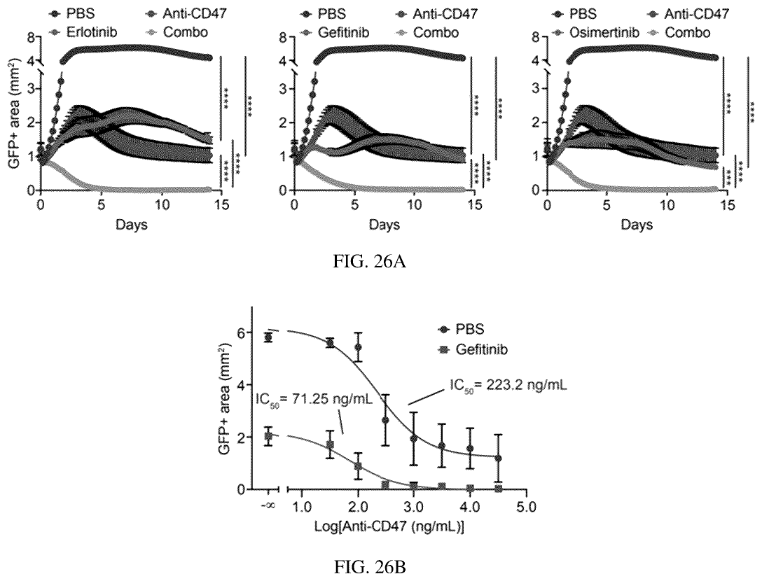

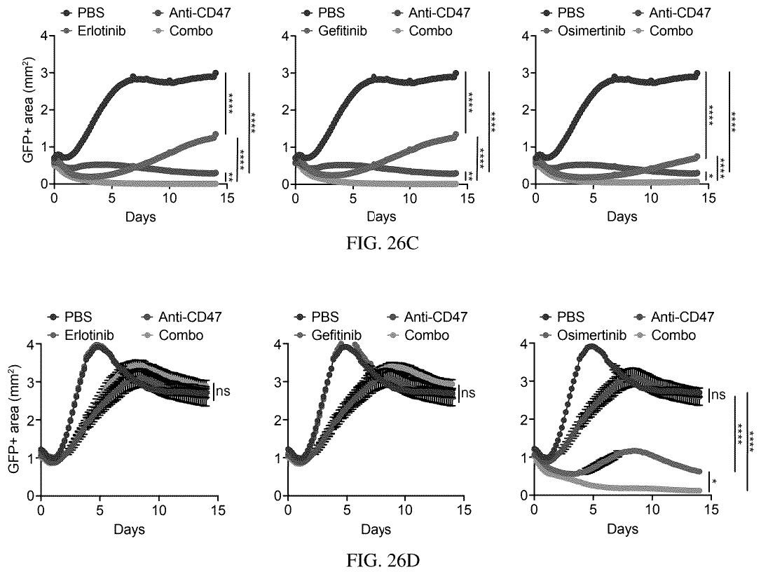

To model the interactions between macrophages and cancer cells as they occur over extended durations of time, we developed ‘long-term’ co-culture assays in which GFP+ cancer cells are co-cultured with primary human macrophages and drug treatments for up to 14 days. We performed whole-well imaging over the co-culture period, and the GFP+ area is quantified over time as a metric of cancer cell growth or death. This experimental system integrates all possible mechanisms of macrophage-mediated cytotoxicity and can evaluate persister cell formation in response to targeted therapies. Using these assays, we co-cultured GFP+ PC9 cells with vehicle control, EGFR TKIs (erlotinib, gefitinib, or osimertinib), anti-CD47, or the combination of anti-CD47 and an EGFR TKI (FIGS. 16A-C, 25A-D, and 26A-B). At baseline, macrophages exerted no substantial anti tumor effect on the PC9 cells. Each individual TKI was able to inhibit the growth of the PC9 cells by themselves, but persister cells always formed and accounted for ˜15% of the cells after 14 days. Treatment with an anti-CD47 antibody caused the formation of patches or foci of cancer cells that remained but also was not able to fully eliminate all cancer cells from the well. However, the combination of any EGFR TKI with an anti-CD47 antibody dramatically eliminated cancer cells and prevented development of persister cells (FIGS. 16A-C, 25A-D, and 26A-B). These effects were observed over a range of concentrations, with the IC₅₀ of the anti-CD47 antibody improving from 223.2 ng/ml (95% CI 158.2-317.3) to 71.25 ng/ml (95% CI 52.39-97.22) upon combination with gefitinib (FIG. 26B). We observed similar effects using GFP+ MGH119-1 cells, a patient-derived EGFR mutant lung cancer cell line (FIGS. 16D and 24C). Again, the combination of each TKI with an anti-CD47 antibody elicited the greatest antitumor response and eliminated or prevented the formation of persister cells.

To understand whether the effects of the combination therapy were dependent on sensitivity to a particular TKI, we also tested GFP+ MGH134-1 cells, a patient-derived cell line with a secondary EGFR1790M mutation. This cell line is consequently resistant to erlotinib and gefitinib but sensitive to osimertinib. In co-culture assays, only osimertinib rendered the cancer cells more vulnerable to the anti-CD47 antibody, whereas neither erlotinib or gefitinib inhibited cell growth as single agents nor in concert with CD47 blockade (FIGS. 16E and 26D). These findings suggest that disruption of oncogenic signaling from EGFR is required for enhanced macrophage-mediated cytotoxicity in response to anti-CD47 therapy. Furthermore, they indicate that the TKIs are acting on the cancer cells rather than exerting off-target effects to the macrophages.

We next evaluated whether targeting CD47 was unique compared to other reported macrophage-directed therapies. We tested a panel of antibodies to macrophage immune checkpoints including CD47, CD40, PD-L1, and CD24. We found that as single agents, only the anti-CD47 antibody and an anti-CD40 agonist antibody were able to induce significant macrophage-mediated cytotoxicity of GFP+ PC9 cells (FIG. 16F). When combined with osimertinib, the anti-CD47 antibody elicited the greatest anti-tumor response and fully eliminated persister cells (FIG. 16F).

### The Efficacy of the Combination Therapy Extends to Lung Cancers With Other Alterations in the RTK-MAPK Pathway

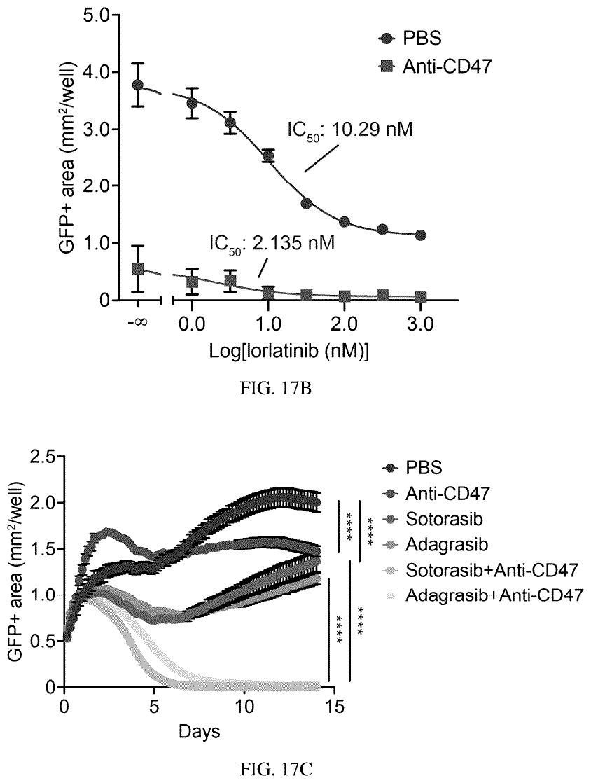

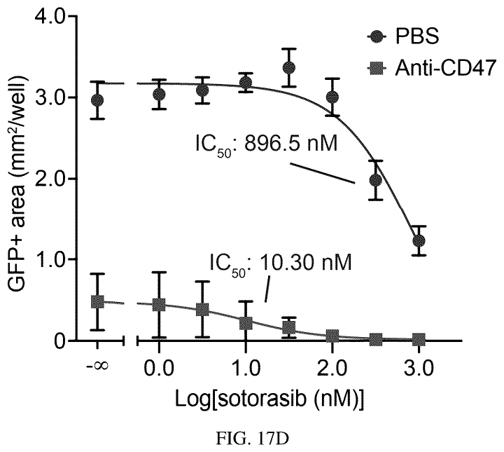

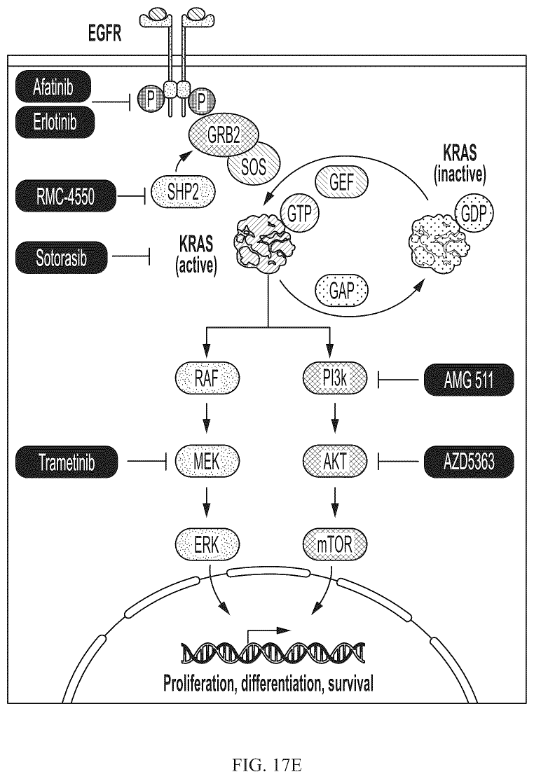

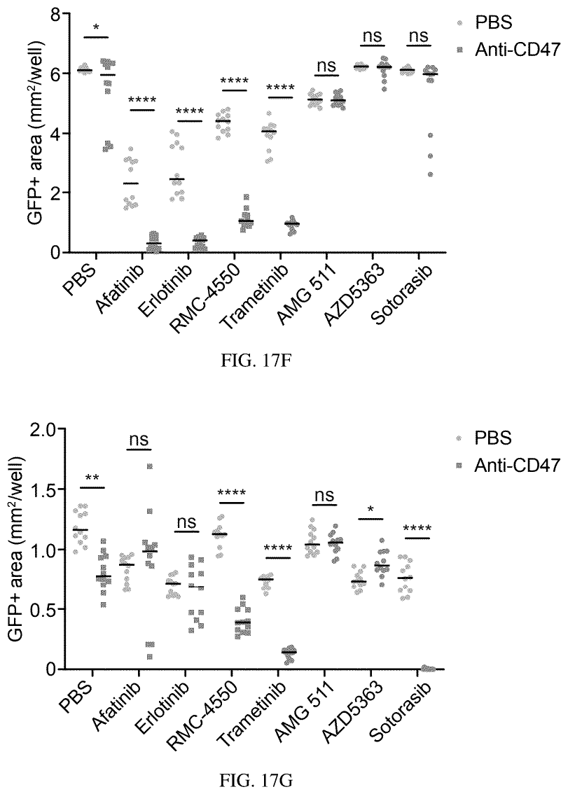

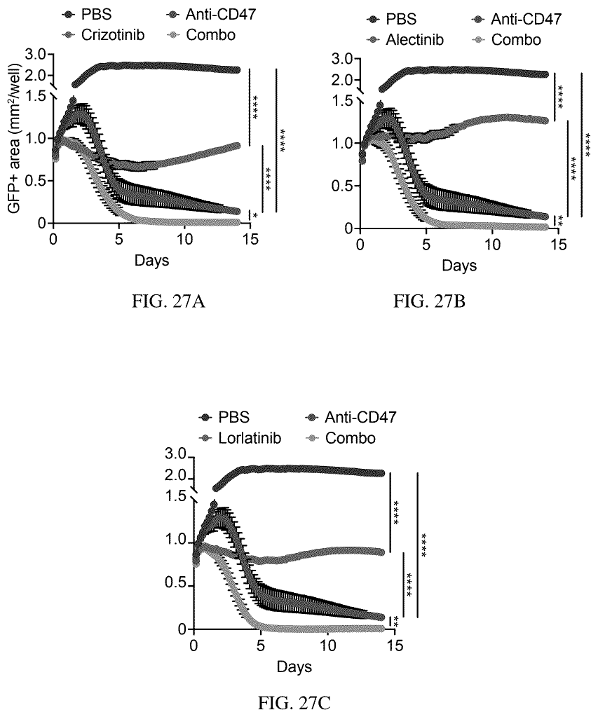

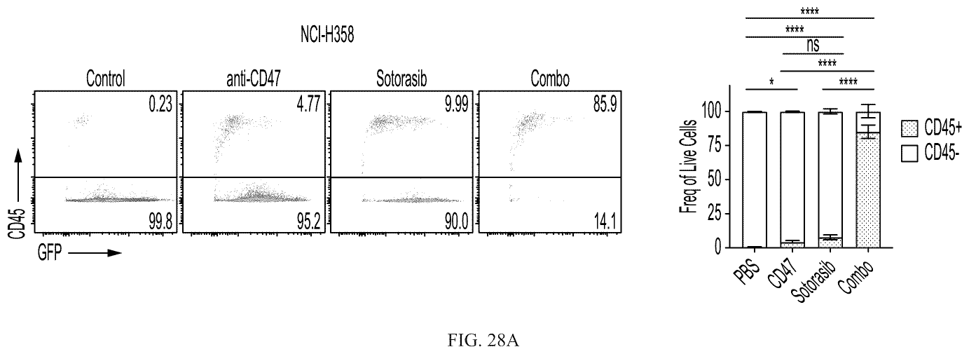

Our results indicate that for EGFR mutant lung cancer, disabling signals from EGFR makes the cells more vulnerable to macrophage-mediated destruction. We reasoned that our findings could also apply to lung cancers containing other types of driver mutations. We therefore examined GFP+ NCI-H3122 cells, which contain an oncogenic EML4-ALK fusion. In co-culture with macrophages, the anti-CD47 therapy significantly impaired the growth of the cancer cells as a single agent (FIGS. 17A and 27A-C). ALK-specific TKIs (crizotinib, alectinib, or lorlatinib) also impaired the growth of the cancer cells as single agents, but a substantial number of persister cells remained in culture. However, the combination of an ALK inhibitor with an anti-CD47 antibody yielded the greatest anti-tumor response, effectively eliminating all cancer cells from the culture (FIGS. 17A and 27A-C). These effects occurred with a dose-response relationship, with the IC50 of lorlatinib decreasing from 10.29 nM (95% CI 8.665-12.22) to 2.135 nM (95% CI 0.6934-6.261) with the combination of an anti-CD47 antibody (FIG. 17B).

Similarly, we investigated cell lines with mutations in KRAS, one of the most commonly mutated genes in lung cancer. We performed co-culture assays using macrophages and NCI-H358 cells, a human lung cancer cell line containing a KRAS^{G12C} activating mutation. In long-term co-culture assays, we found that anti-CD47 antibodies or KRAS^{G12C} inhibitors (sotorasib or adagrasib) inhibited cancer cell growth over time, but generally had only moderate effects as single agents (FIG. 17C). In contrast, the combination of the two therapies had a striking effect with dramatic elimination of tumor cells from culture (FIG. 17C). As above, we performed titrations of sotorasib and determined that a dose-response relationship existed and that the combination therapy substantially decreased the IC₅₀ of sotorasib (896.5 nM, 95% CI 558.6, 1697 nM; versus 10.30 nM, 95% CI 2.949, 40.48 nM) (FIG. 17D).

Since EGFR and other receptor tyrosine kinases (RTKs) can transduce growth signals via the MAPK pathway and/or the PI3K/AKT pathway, we tested a panel of inhibitors to dissect the molecular mediators underlying the effects of the combination therapy (FIG. 17E). Using co-culture assays with EGFR mutant PC9 cells, we found that any active inhibitor of the MAPK pathway could be enhanced by combination with an anti-CD47 antibody, whereas no significant enhancement was observed when combining an anti-CD47 antibody with AKT or PI3K inhibitors (FIG. 17F). Similarly, using KRASG12C mutant NCI-H358 cells, we found that inhibition of SHP2, KRAS^{G12C}, or MEK could be enhanced by combining with anti-CD47 therapy, whereas no significant enhancement was observed when combining with PI3K or AKT inhibitors (FIG. 17G). These findings suggest that efficacy of the combination therapy is specifically dependent on inhibition of the MAPK pathway rather than alternative signaling pathways.

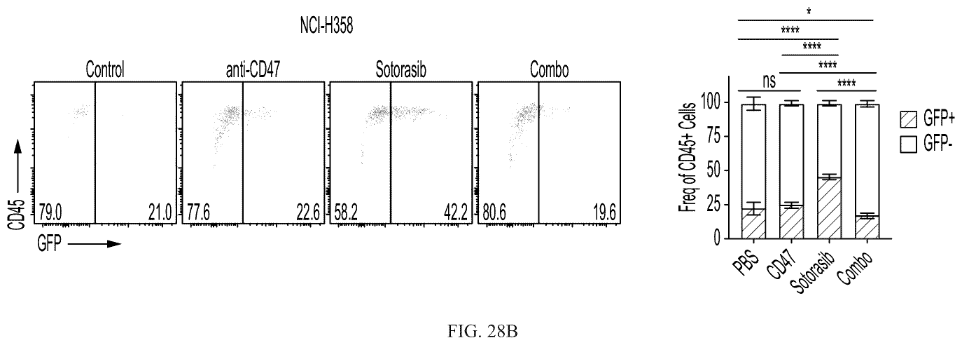

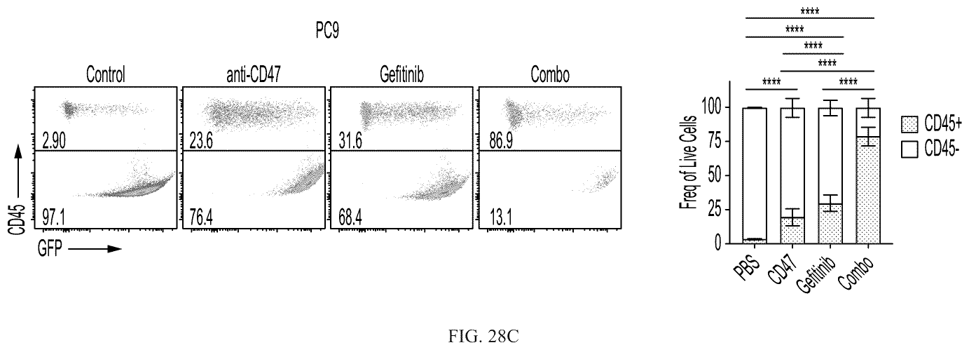

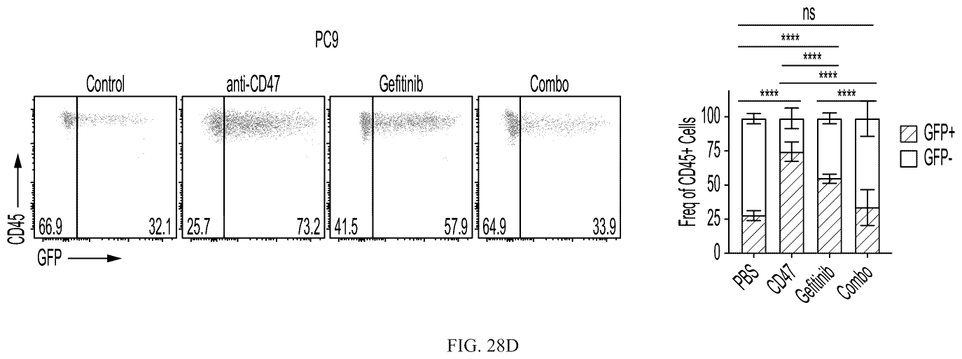

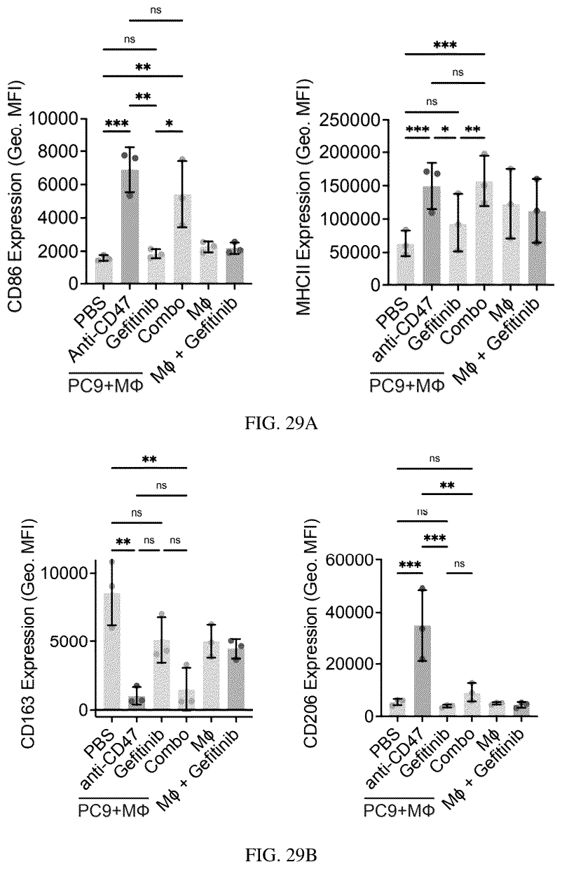

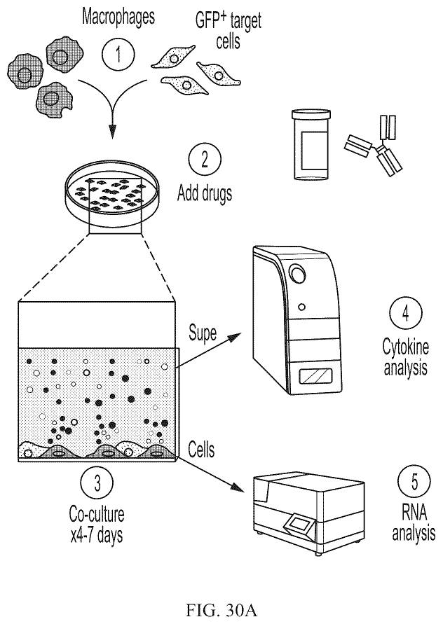

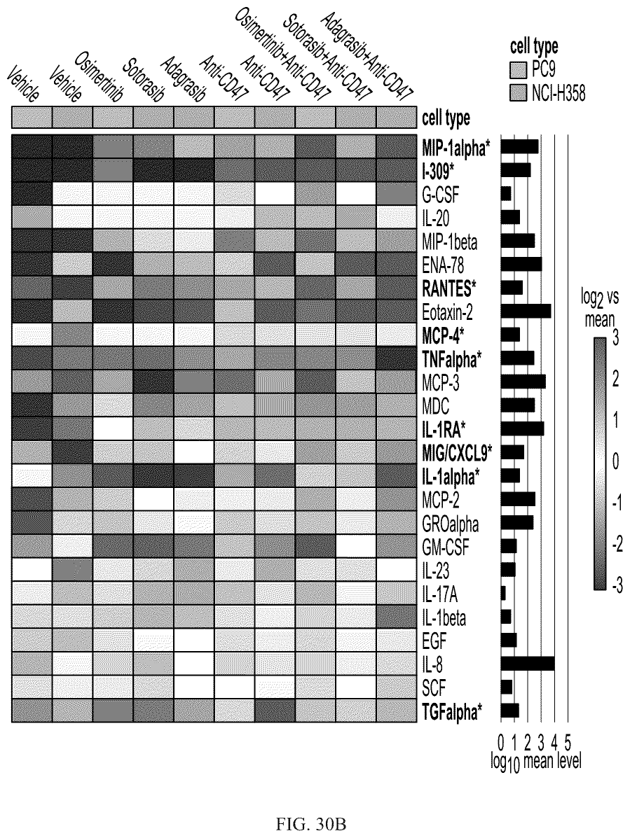

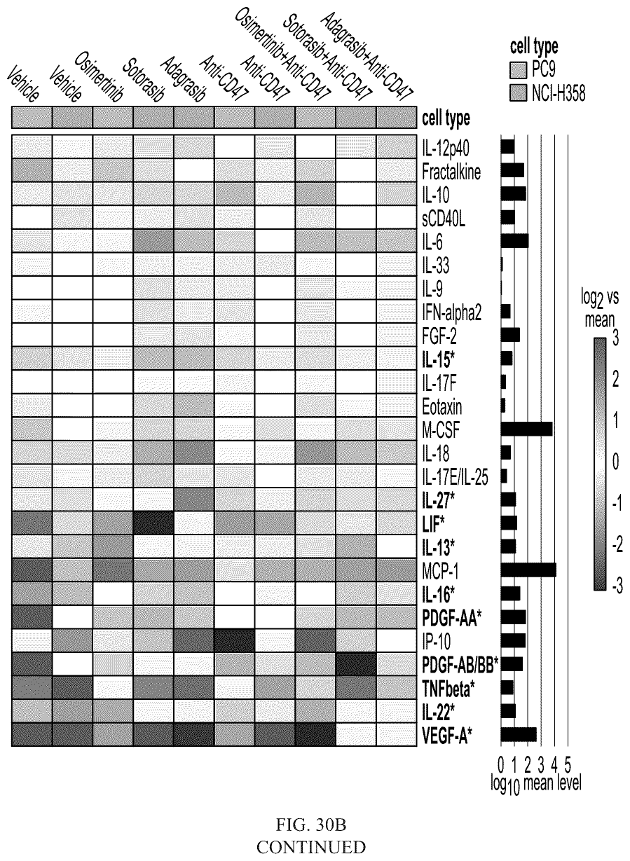

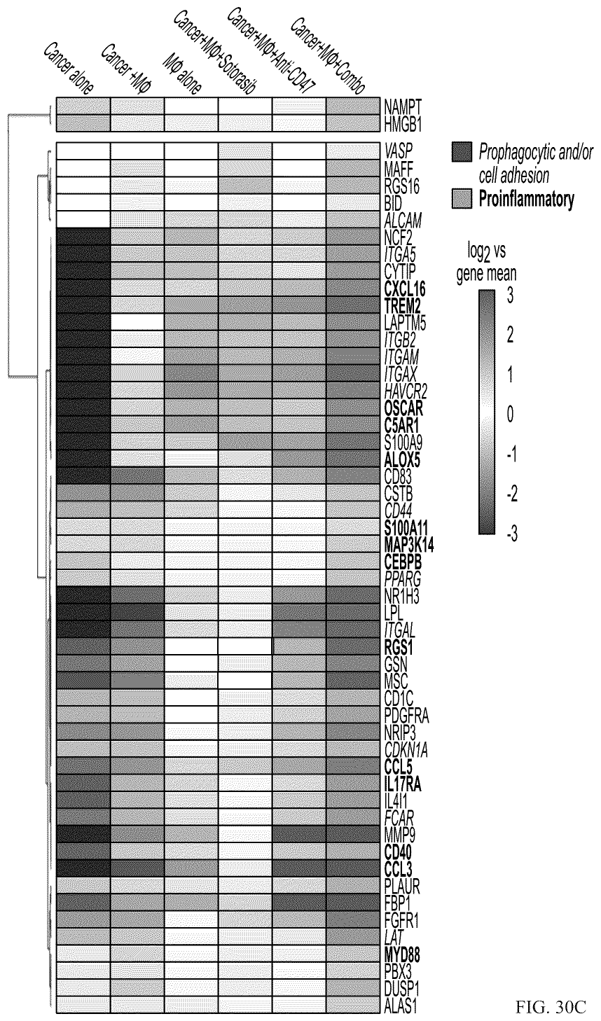

To understand changes in macrophage activation states as a consequence of the combination therapy, we performed multiparameter flow cytometry, multiplex cytokine analysis, and transcriptional profiling. In co-culture assays, macrophages exhibited increased phagocytosis in response to either targeted therapies or anti-CD47 antibodies as single agents (FIGS. 28A-D). Treatment with the combination therapy elicited upregulation of the M1 markers CD86 and MHC II with relative downregulation of the M2 markers CD163 and CD206 (FIGS. 29A-B). We also found that the combination of targeted therapies and anti-CD47 elicited a unique pro-inflammatory cytokine signature that included significant increases in MIP-1α, RANTES, MCP-4, and TNFα, and a corresponding decrease in VEGF secretion by the cancer cells (FIGS. 30A-B). Transcriptional profiling identified 50 genes that were significantly upregulated in macrophages upon treatment with the combination therapy. This gene set included a predominance of genes involved in phagocytosis, cell-cell adhesion, and major drivers of inflammation such as MYD88 (FIG. 30C). These profiling experiments emphasize the robust anti-tumor state of the macrophages in response to the combination treatment.

### The Combination of Targeted Therapies and CD47 Blockade is Effective in Mouse Tumor Models Bearing Driver Mutations

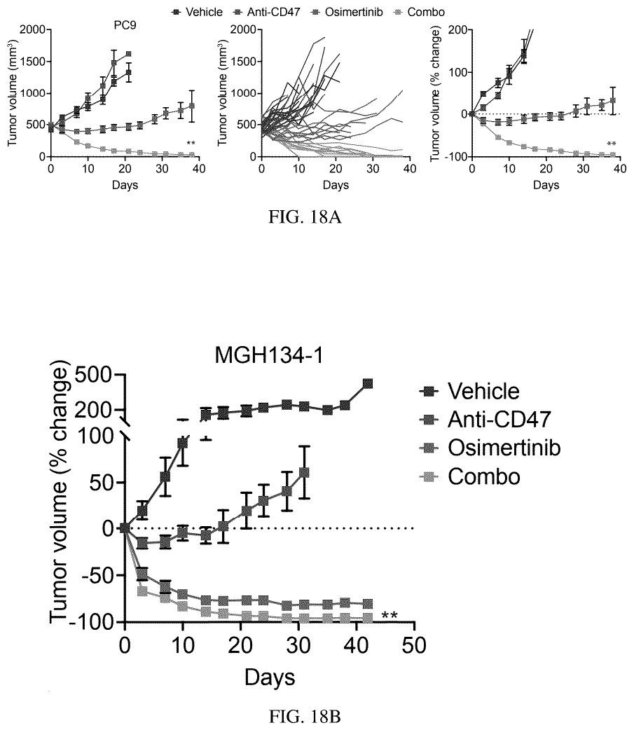

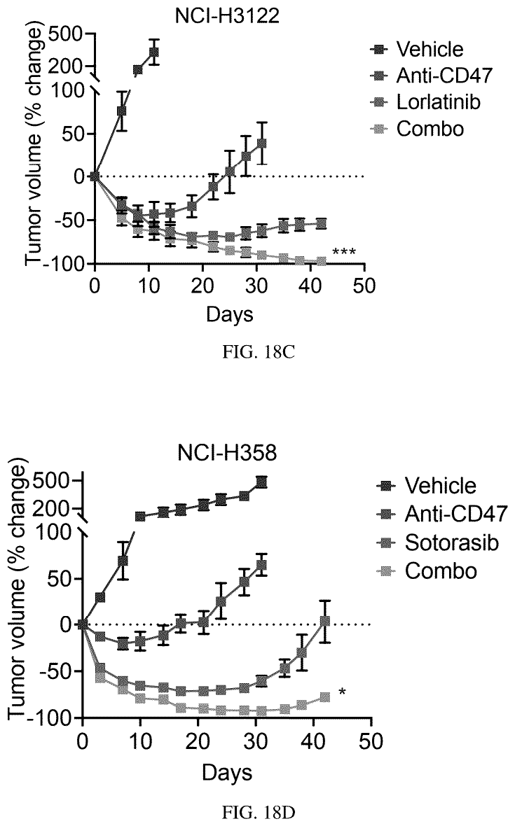

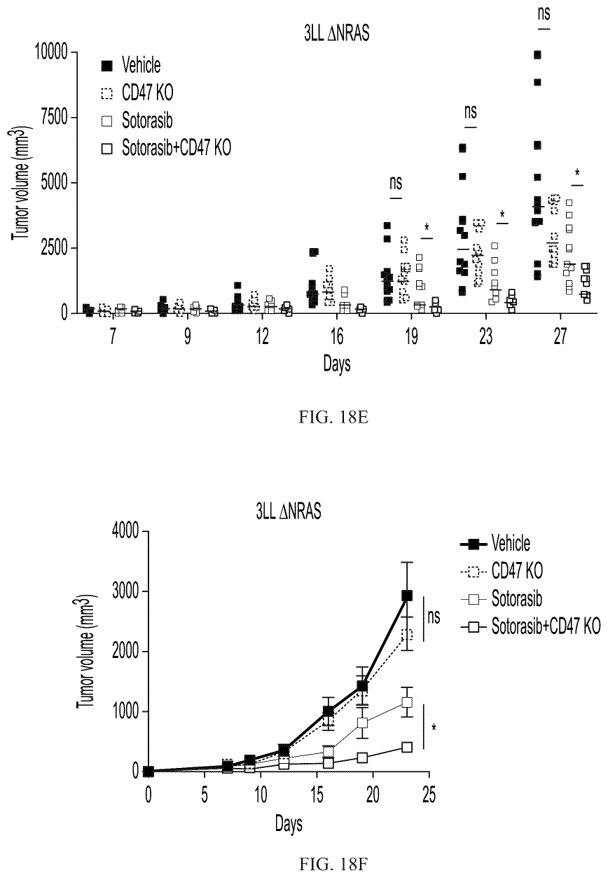

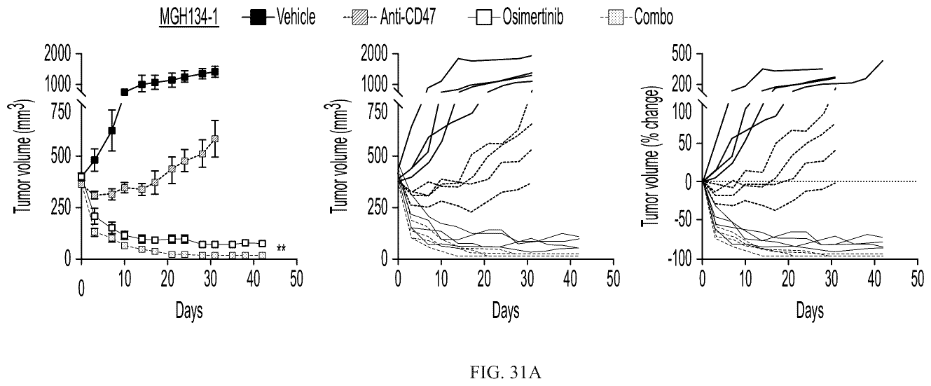

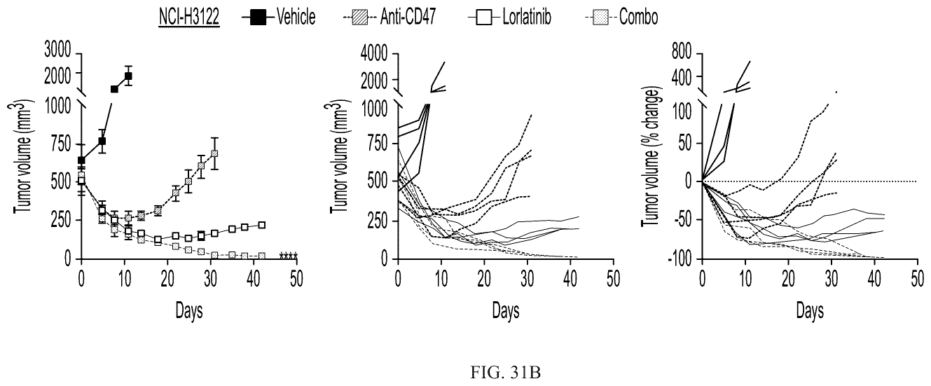

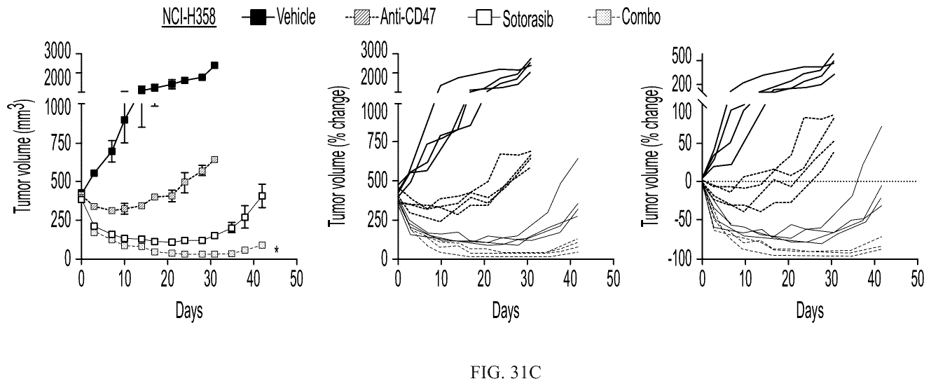

To study the efficacy of the combination therapy in vivo, we first employed xenograft models of human lung cancer. We used NSG mice, which lack functional T-, B- and NK cells but contain macrophages that can be stimulated to attack tumors. Importantly, NSG mice have an allele of SIRPα that cross-reacts with human CD47. therefore they have been used as a gold-standard model for evaluating CD47-blocking therapies in vivo. We engrafted mice subcutaneously with PC9 cells and allowed tumors to grow to ˜500 mm³. Mice were then randomized to treatment with vehicle control, an anti-CD47 antibody, osimertinib, or the combination of osimertinib and the anti-CD47 antibody (FIG. 18A). As a single agent, the anti-CD47 antibody produced no significant inhibition of tumor growth. Treatment with osimertinib as a single agent was able to inhibit tumor growth, but tumors gradually progressed over time. Remarkably, treatment with the combination therapy dramatically reduced tumor burden and elicited complete elimination of tumors in several animals (FIG. 18A). We also tested a patient-derived xenograft model of EGFR mutant lung cancer (MGH134-1), and again observed the greatest anti-tumor effects from the combination treatment of osimertinib with an anti-CD47 antibody (FIGS. 18B and 31A-C).

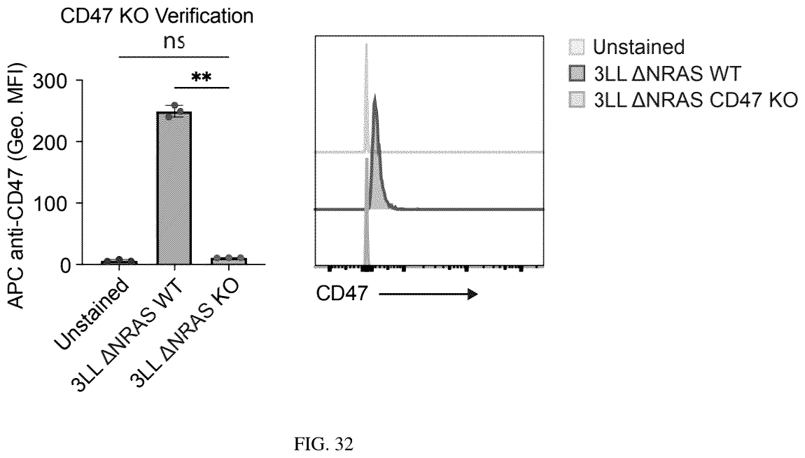

To understand whether these findings could extend to other types of lung cancer bearing different driver mutations, we tested models of ALK-positive lung cancer (NCI-H3122 cells) and KRAS^{G12C} mutant lung cancer (NCI-H358 cells). In each of these models, the greatest antitumor effects were observed with the combination of targeted therapy (lorlatinib for NCI-H3122 cells, sotorasib for NCI-H358 cells) and an anti-CD47 antibody (FIGS. 18C-D and 31A-C), consistent with our observations in vitro. Similarly, we tested an immunocompetent, syngeneic model of KRAS^{G12C} mutant lung cancer using 3LL ΔNRAS cells, a variant of Lewis lung carcinoma that harbors an endogenous KRAS^{G12C} mutation and responds to KRAS inhibitors. In this model, we found that genetic ablation of CD47 had no significant effect on tumor growth by itself (FIGS. 18F and 32). Sotorasib was able to inhibit tumor growth as a single agent, yet the greatest inhibition of tumor growth occurred upon sotorasib treatment of a CD47 knockout cell line (FIG. 18F). Together, these findings indicate that our in vitro findings translate to in vivo models and that dual blockade of CD47 and oncogenic drivers can enhance anti-tumor responses to lung cancer.

### β2-Microglobulin and CD73 are “Don't Eat Me” Signals That Can be Altered by Targeted Therapies

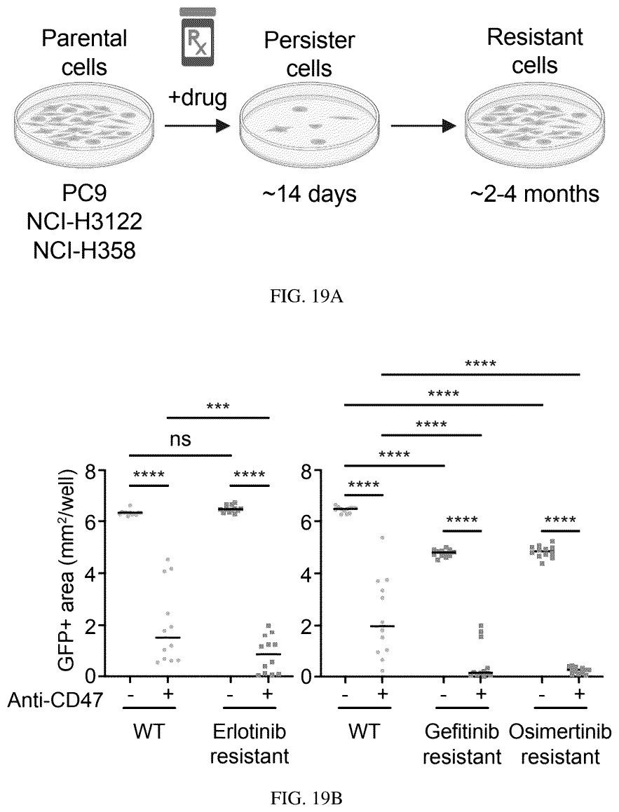

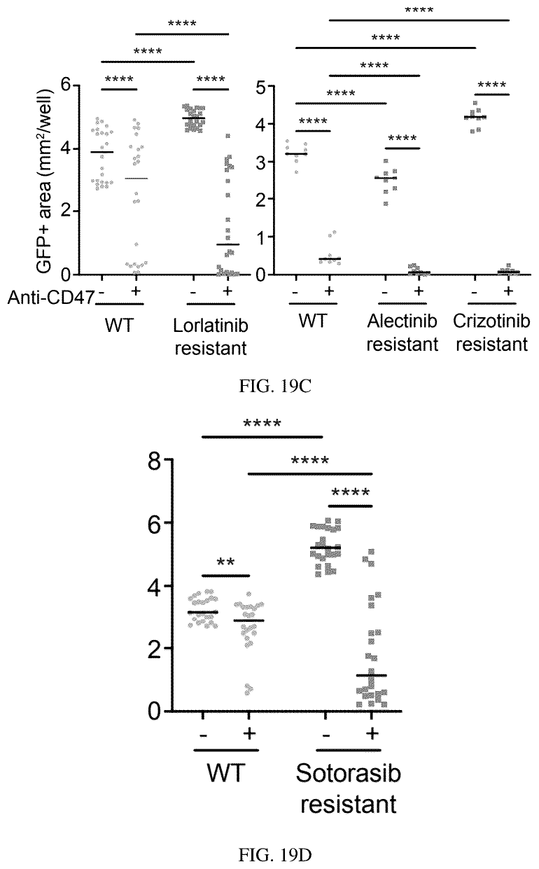

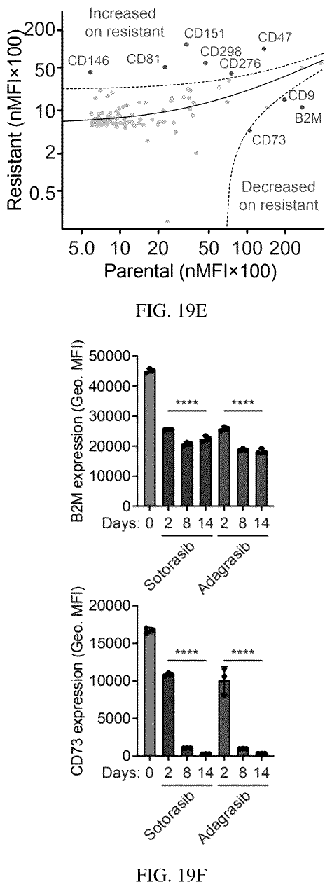

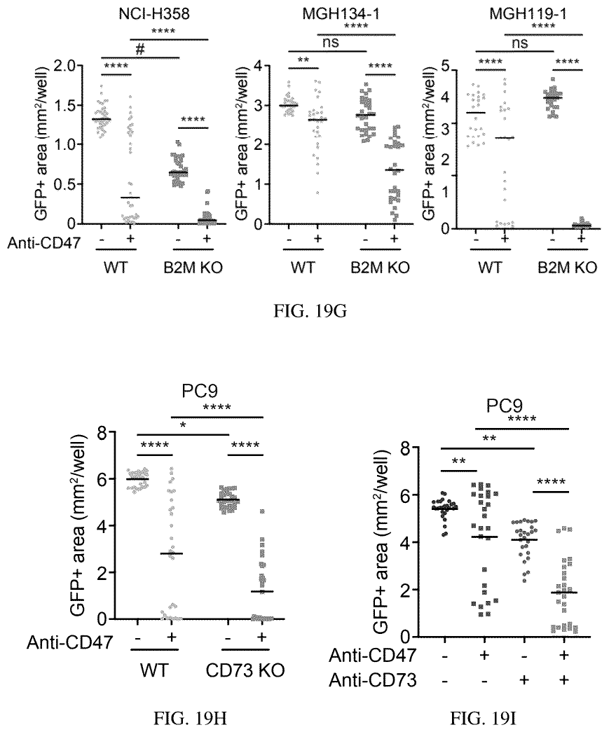

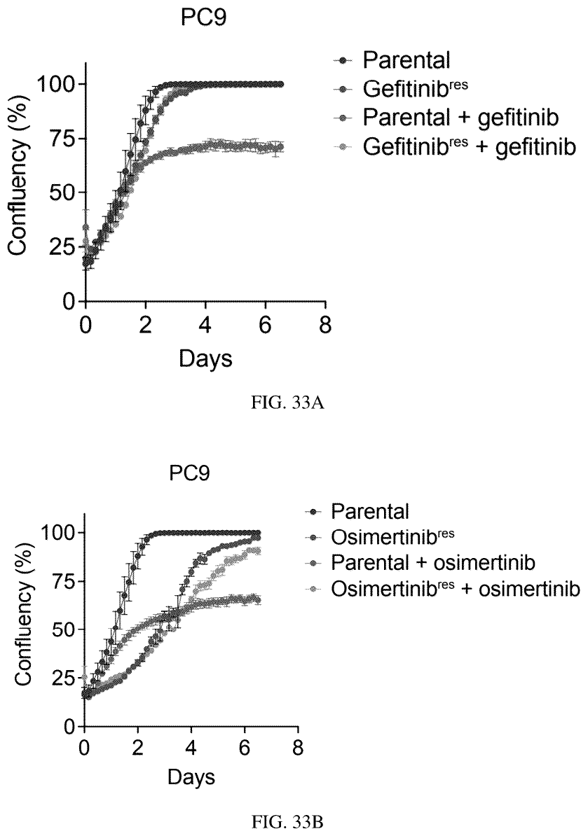

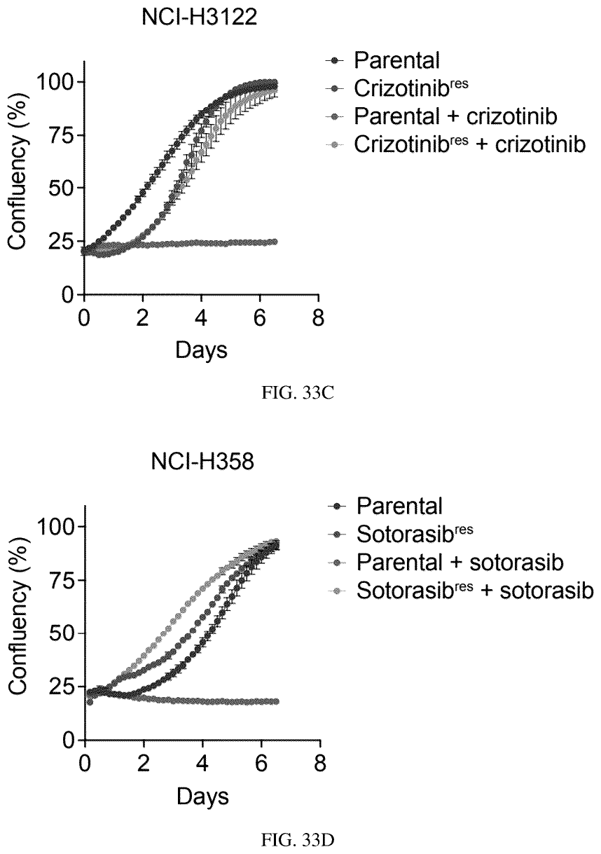

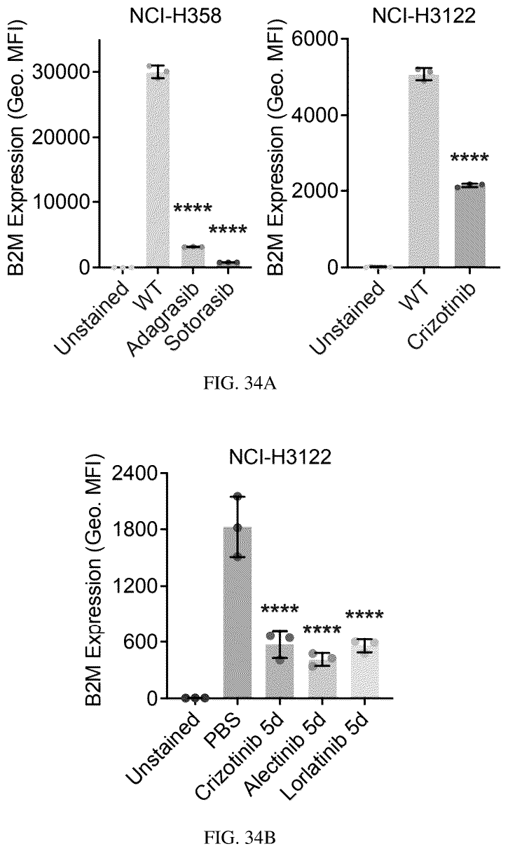

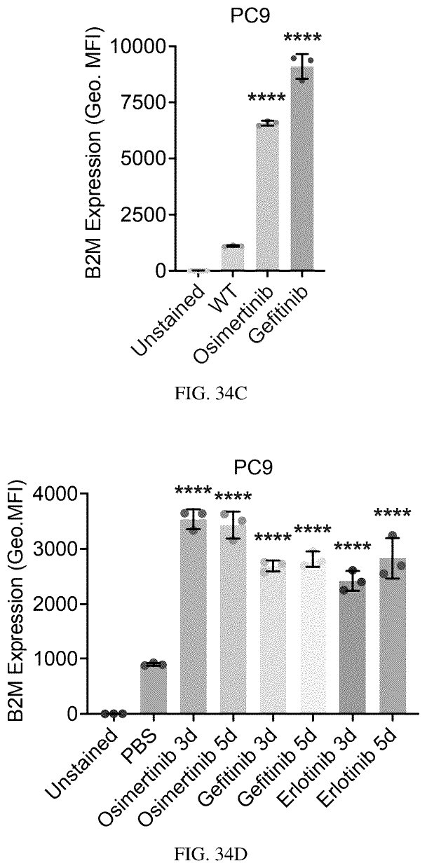

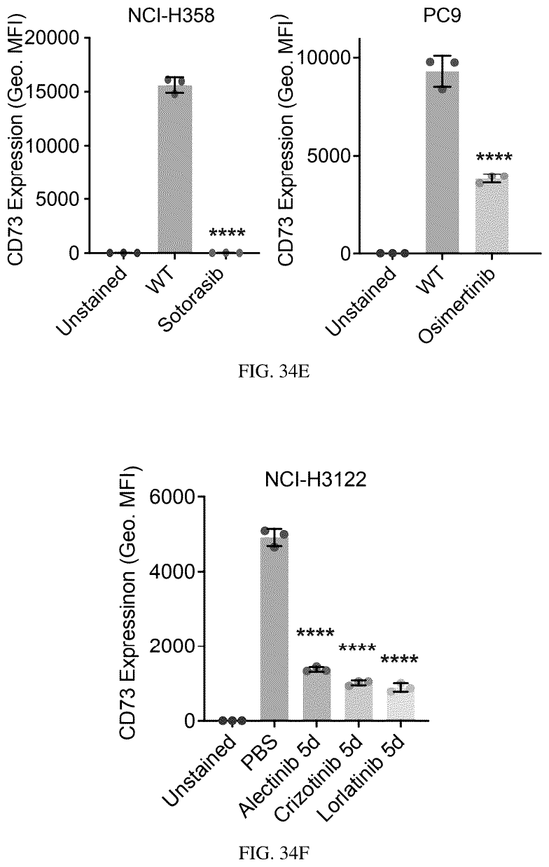

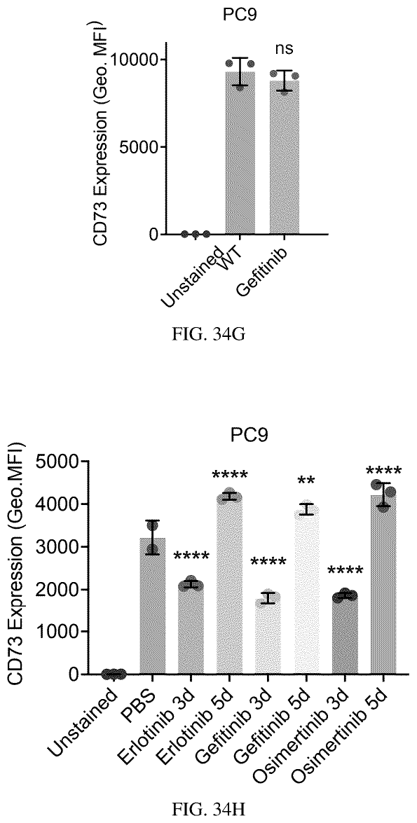

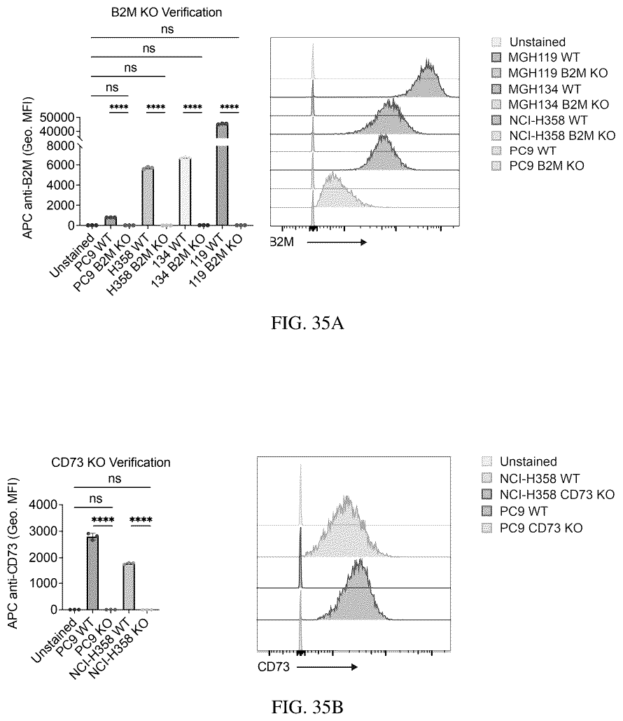

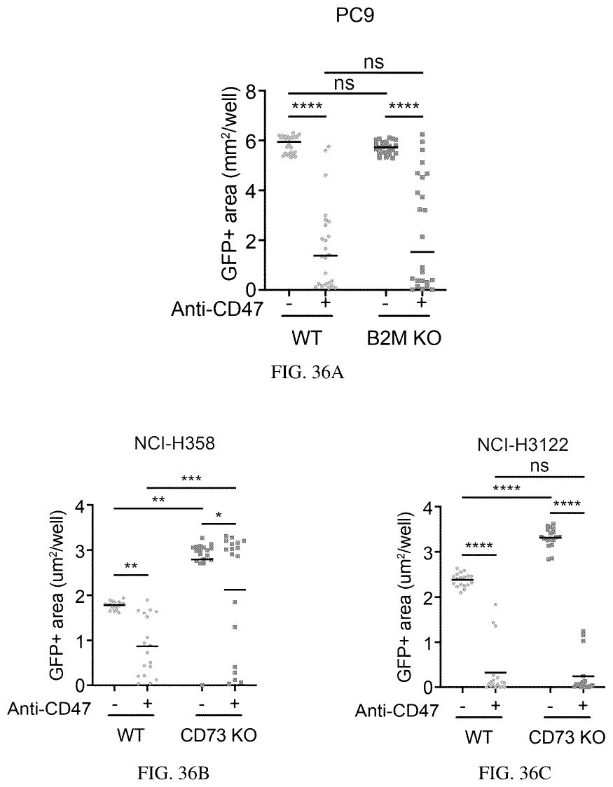

To understand the mechanisms by which targeted therapies make cancer cells more vulnerable to macrophage-directed therapies, we generated a panel of seven GFP+ lung cancer cell lines that are each resistant to their respective targeted therapies. The cell lines were generated by prolonged culture in 1 uM of appropriate targeted therapy until resistant cells emerged and grew at rates comparable to their naive parental counterparts (FIGS. 19A and 33A-D). The lines included PC9 cells (resistant to erlotinib, gefitinib, or osimertinib), NCI-H3122 cells (resistant to crizotinib, alectinib, or lorlatinib), and NCI-H358 cells (resistant to sotorasib). As a consequence of becoming drug resistant, we found that each cell line also became more sensitive to macrophage-mediated cytotoxicity in response to anti-CD47 therapy (FIGS. 19B-D). We hypothesized that changes in cell-surface proteins likely mediated this effect, since these proteins are required for intercellular interactions between macrophages and cancer cells. Therefore, we performed comprehensive surface immunophenotyping of naïve parental versus sotorasib-resistant NCI-H358 cell lines to identify differentially expressed surface antigens (FIG. 19E). We found two known immunoinhibitory factors, β2-microglobulin (B2M) and CD73, that were substantially downregulated on the sotorasib-resistant line. Moreover, we found that B2M and CD73 were significantly downregulated on additional resistant cell lines, and could be downregulated as a consequence of initial treatment with targeted therapies (FIGS. 19F and 34A-H). To evaluate the functional contributions of these surface proteins, we generated knockout cell lines using CRISPR/Cas9 (FIGS. 35A-B). We found that genetic deletion of B2M could influence macrophage killing in response to anti-CD47 therapy for the majority of cell lines tested (FIGS. 19G and 35A-B). In contrast, CD73 knockout seemed to act as a “don't eat me” signal only for PC9 cells, and a CD73-blocking antibody was also effective in this setting (FIGS. 19H-I and 36A-C). These findings indicate both B2M and CD73 can act as functional macrophage immune checkpoints for lung cancers with driver mutations, and that their downregulation can contribute to vulnerability to macrophage attack. Importantly, individual cancer specimens may differentially rely on these distinct immune checkpoints to evade macrophage-mediated cytotoxicity.

### Discussion

To identify therapies that sensitize cancer cells to macrophage-mediated destruction, we performed an unbiased, cell-based functional screen of 800 FDA-approved drugs using primary human macrophages as effectors. We identified genotype-directed targeted therapies as those that prime lung cancer cells for macrophage-mediated destruction. In subsequent in vitro and in vivo validation studies, we found these results extended to multiple NSCLC models harboring diverse oncogenic driver alterations treated with their corresponding targeted therapies. The effects of the combination therapy may be unique to inhibition of the RTK-MAPK pathway, as similar enhancement was not observed as a class effect of chemotherapy drugs in our screen or when using AKT or PI3K inhibitors. Our findings have immediate translational implications since they suggest the combination of targeted therapies and CD47-blocking therapies could be a superior strategy for treating patients with lung cancers with driver mutations. Furthermore, we also demonstrate that cell-intrinsic resistance to targeted therapies can cross-sensitize to CD47 blockade.

To date, clinical trials combining targeted therapies and T cell-directed immune checkpoint inhibitors have not been successful for lung cancer. These studies have either been limited in efficacy or demonstrated excessive toxicity. As an example, the combination of osimertinib with an anti-PD-L1 antibody showed severe side effects in nearly 50% of patients (Oxnard G R, et al. TATTON: a multi-arm, phase Ib trial of osimertinib combined with selumetinib, savolitinib, or durvalumab in EGFR-mutant lung cancer. Ann Oncol. 2020; 31(4):507-16). Macrophage-directed therapies are an orthogonal treatment modality and may benefit different patients than T cell-directed therapies. This is particularly true since macrophages have inherent ability to kill cancer cells when provided with an appropriate stimulus, whereas T cell cytotoxicity is intertwined with the tumor mutational burden and the presence of neoantigens. In addition, we found that downregulation of B2M and CD73 contribute to enhanced sensitivity to macrophage killing. B2M is required for MHC class I expression on the cell surface, which CD8 T cells depend on for antigen recognition. However, B2M also acts as a “don't eat me” signal by binding to LILRB1, an inhibitory receptor on macrophages. Downregulation of B2M may decrease antigen presentation to make cancer cells resistant to T cell-directed immunotherapies while simultaneously making them more vulnerable to macrophage attack. Thus, B2M expression may reflect a critical pivot point between innate and adaptive immune activation. Similarly, CD73 is an ectoenzyme that catalyzes the breakdown of AMP to immunosuppressive adenosine in the tumor microenvironment. Its downregulation may make lung cancer cells more sensitive to macrophage-mediated cytotoxicity. Our findings suggest lung cancer specimens may differentially rely on these immunosuppressive signals to evade detection by macrophages. The high-throughput screening platform we developed is a robust system to identify drugs that activate or inhibit macrophage-mediated cytotoxicity.

### Cell Lines

PC9, NCI-H3122, NCI-H358 cells were obtained from the Hata Laboratory (Massachusetts General Hospital). NSCLC patient-derived cell lines and specimens were provided by the Hata Laboratory via the Massachusetts General Hospital and collected under an IRB-approved protocol. Human NSCLC cell lines were cultured in in RPMI (Thermo Fisher) supplemented with 10% ultra-low IgG fetal bovine serum (Thermo Fisher), 100 units/mL penicillin, 100 ug/mL streptomycin, and 292 ug/mL L-glutamine (Thermo Fisher). 3LL ΔNRAS cells were provided by the lab of Dr. Julian Downward (The Francis Crick Institute, London, UK) and were cultured in DMEM (Thermo Fisher) supplemented with 10% ultra-low IgG fetal bovine serum, 100 units/mL penicillin, 100 ug/mL streptomycin, and 292 ug/mL L-glutamine. Cell lines were maintained in humidified incubators at 37 degrees C. with 5% carbon dioxide.

GFP+ lines were generated by lentiviral transduction of cell lines using CMV-GFP-T2A-Luciferase pre-packaged virus (Systems Bio). Transduced cells were then sorted for stable GFP expression.

Genetic modification of cell lines: Knockout cell lines were generated by CRISPR/Cas9-mediated genome editing. A CD47 knockout variant of 3LL ΔNRAS cells was generated using Gene Knockout Kit version 2 targeting murine CD47 (Synthego). Knockout variants of PC9. NCI-H358, MGH119, and/or MGH134 were generated using Gene Knockout Kit version 2 targeting human B2M or human CD73 (Synthego). Gene knockout was performed via ribonucleoprotein transfection with recombinant Cas9 (Synthego). Cells were then stained for surface antigen expression and sorted using a FACSAria II (BD Biosciences) to generate negative cell lines. For murine CD47, staining was performed using APC anti-murine CD47 clone miap301 (BioLegend) and sorting was used to generate a clonal population. For human lines, staining wass performed with APC anti-human B2M clone 2M2 (Biolegend) or APC antihuman CD73 clone AD2 (Biolegend) and used to sort polyclonal lines that were negative for surface antigen expression.

### Ex Vivo Generation of Primary Human Macrophages

Primary human macrophages were generated from peripheral blood mononuclear cells as previously described (Weiskopf et al Science. 2013; 341(6141):88-91). Briefly, leukocyte reduction chambers were obtained from healthy human donors from discarded apheresis products via the Crimson Core Biobank (Boston, MA). Monocytes were labeled using StraightFrom Whole Blood CD14 MicroBeads (Miltenyi Biotec) and purified using an autoMACS Pro Separator (Miltenyi Biotec). Monocytes were then cultured in IMDM (Thermo Fisher) supplemented with 10% ultra-low IgG fetal bovine serum, 100 units/mL penicillin, 100 ug/mL streptomycin, and 292 ug/mL L-glutamine and 20 ng/ml human M-CSF (Peprotech) for at least 7 days. Cells were passaged or replated as necessary and typically maintained in culture for 2-4 weeks.

### FDA Drug Library Screen

GFP+ PC9 cells and primary human macrophages were co-cultured in 384-well plates in IMDM supplemented with 10% ultra-low IgG fetal bovine serum, 100 units/mL penicillin, 100 ug/mL streptomycin, and 292 ug/mL L-glutamine and 20 ng/mL human M-CSF. Purified anti-CD47 antibody clone B6H12 (BioXCell or eBioscience) was added at a working concentration of 10 ug/mL. Duplicate control plates were plated with GFP+ PC9 cells alone. A curated library of 800 FDA-approved drugs was transferred to the plates via Echo Acoustic Liquid Handler (Koch Institute High-Throughput Sciences Facility). Cells were then incubated for 3-5 days. Wells were imaged using automated fluorescence microscopy with an Incucyte S3 system (Sartorius). Automated image analysis was performed using Incucyte Analysis Software (Sartorius) to quantify the percentage of GFP+ area occupied by target cells in each well. To assess macrophage-dependent cytotoxicity, GFP+ area in experimental wells (containing GFP+ PC9 cells, macrophages, and anti-CD47 antibodies) was normalized to GFP+ area in control wells (containing GFP+ PC9 cells alone). Library screens were performed in five independent times, each using an individual macrophage donor (n=5 experiments). For individual compounds, p-values were computed using a one-sample t test after controlling for row, column, and plate effects

### FACS Analysis

Analysis of cell surface antigens was performed using live cells in suspension. Cells were blocked with 100 ug/mL mouse IgG (Lampire Biological Laboratories) or Human TruStain FcX (BioLegend). The following antibodies were used for FACS analysis: APC-conjugated anti-CD47 clone B6H12 (Thermo Fisher), Alexa Fluor 647-conjugated anti-HLA-A,B,C (MHC class I) clone W6/32 (BioLegend), APC-conjugated anti-PD-L1 clone 29E.2A3 (BioLegend), Alexa Fluor 647-conjugated anti-CD24 clone ML5 (BioLegend), PE-conjugated anti-CALR clone FMC 75 (Abcam). APC anti-CD45 clone 2D1 or clone HI30 (BioLegend). Viability of cell lines was assessed by staining with 100 ng/ml DAPI (Milipore Sigma). For analysis of primary pleural fluid specimens, Alexa Fluor 488-conjugated anti-EpCam clone 9C4 (BioLegend) was used to mark the malignant cell population. Cells were analyzed using an LSR Fortessa equipped with a High Throughput Sampler (BD Biosciences).

### Targeted Therapies

The following targeted therapies were used in this study: erlotinib, gefitinib, osimertinib, crizotinib, alectinib, lorlatinib, sotorasib, adagrasib (Selleckchem). MAPK pathway inhibitors included: afatinib, erlotinib, RMC-4550, sotorasib, trametinib, AMG 511, AZD5363 (Selleckchem).

### Phagocytosis Assays

In vitro phagocytosis assays were performed as previously described (Weiskopf et al Science. 2013; 341(6141):88-91). Briefly, GFP+ PC9 cells or CFSE+ PC9 cells were used as target cells. Labeling with CFSE (Thermo Fisher) was performed according to manufacturer's instructions. Target cells were exposed to EGFR TKIs (erlotinib, gefitinib, or osimertinib) for 24-72 hours. Live, adherent cells were then collected, washed. and co-cultured with primary human macrophages at a target: macrophage ratio of 4:1. Cells were co-cultured in the presence or absence of 10 ug/mL purified anti-CD47 antibody clone B6H12. Cells were co-cultured for 2 hours in serum-free IMDM in round-bottom ultra-low attachment 96-well plates (Corning). After the incubation period, cells were washed and analyzed by flow cytometry. Macrophages were identified using APC anti-CD45 clone 2D1 or clone HI30 and target cells were identified by CFSE or GFP fluorescence. Phagocytosis was quantified as the percentage of macrophages that contained CFSE or GFP+signal. Phagocytosis was normalized to the maximal response by each independent macrophage donor. Dose-response curves were generated using Prism version 9.2.0 (GraphPad).

### Long-term Co-Culture Assays

Long-term co-culture of primary human macrophages was performed using GFP+ target cells. Cells were cultured in IMDM supplemented with 10% ultra-low IgG fetal bovine serum, 100 units/mL penicillin, 100 ug/mL streptomycin, and 292 ug/mL L-glutamine and 20 ng/mL human M-CSF. Purified anti-CD47 antibody clone B6H12 was added at a working concentration of 10 ug/mL. Targeted therapies were added at a final working concentration of 1.0 uM except as otherwise indicated. Cells were then co-cultured for 7-14 days with whole-well imaging of phase contrast and GFP channels performed every 4 hours using an Incucyte S3 system. Automated imaging analysis was performed using Incucyte Analysis Software. Images taken on day 6.5 was used as a standard reference time point for data comparison and statistical analysis. For dose-response experiments, sigmoidal dose-response curves were generated using Prism version 9.2.0 (GraphPad) to determine IC50 parameters.

### Macrophage Immune Checkpoint Experiment

In long-term assays to assess macrophage immune checkpoint targeting, the following macrophage-directed therapeutics were used: purified anti-CD47 clone B6H12, anti-CD40 clone G28.5 (BioXCell), anti-hPD-L1-hlgG1 (Invivogen), anti-hPD-L1-hIgG1 (N298A) (Invivogen), purified anti-CD24 clone SN3 (GeneTex), and purified anti-CD73 clone AD2 (BioLegend).

### In Vivo Treatment Models

NOD.Cg-Prkdc^{scid} I12rg^{tm1Wj1}/SzJ (NSG) mice (Jackson Laboratory) were used for all xenograft tumor models. For xenograft models, mice were engrafted with human cancer cells in the subcutaneous tissue of the flank. Tumors were measured regularly by caliper and mice were randomized to treatment cohorts when tumor volumes reached approximately 500 mm³. Drugs used for treatment included: osimertinib (5 mg/kg via oral administration five times per week), lorlatinib (6 mg/kg via oral administration five times per week), sotorasib (100 mg/kg via oral administration five times per week), or anti-CD47 antibody clone B6H12 (250 ug via intraperitoneal injection three times per week). The mice were euthanized when the tumor size exceeded 20 mm in any one dimension or when mice reached humane experimental endpoints according to the Institutional Animal Care and Use Committee approved protocols.

For a syngeneic, immunocompetent tumor model, C57BL/6 mice (Jackson Laboratory) were engrafted with 3LL ΔNRAS cells or a CD47-knockout variant. Mice were engrafted in the subcutaneous tissue using a dual flank model. Wild-type tumors were engrafted on one flank, and CD47-knockout tumors were engrafted on the contralateral flank. Mice were then randomized to treatment with vehicle control or sotorasib (30 mg/kg via oral administration five times per week). Tumor volumes were measured twice weekly using electronic calipers and calculated using the formula as above.

### Statistics

Data analysis of FDA drug screens was performed across 5 replicates of the experiments as follows. GFP+ area was quantified from all wells as described above. After logarithmic transformation, row, column and plate effects were controlled using Tukey's median polish, after which p-values for individual compounds (FIG. 15I) were computed using a one sample t test. For plate-based time-series measurements (FIG. 15J), “relative area” was defined as the GFP+ area at time t divided by the GFP+ area at time zero, obviating the need for controlling row, column and plate effects. To assess the effect of combining macrophage+anti-CD47 conditions with different classes of drugs (FIG. 15K), each class was compared with control wells (i.e., empty or DMSO) using a two-sample t test with the Benjamini-Hochberg method to control for multiple hypothesis testing.

In general, flow cytometry data was analyzed by comparing geometric mean fluorescence intensity (geometric MFI) of individual surface antigens by one-way ANOVA with Holm-Sidak multiple comparison test. Comprehensive surface immunophenotyping was analyzed from a single set of measurements. Data for naive parental versus sotorasib-resistant lines were uniformly adjusted by linear transformation to correct for negative values. Data were fit using linear regression which was used to define the 95% prediction interval.

In vitro phagocytosis assays were analyzed by comparing the mean percentage of GFP+ macrophages between different conditions. Experiments were performed at least 2 independent trials using a total of 6-8 individual donors except as otherwise indicated. The mean values for relevant comparisons were assessed by two-way ANOVA with Holm-Sidak multiple comparison test using Prism version 9.2.0 (Graphpad).

Long-term co-culture assays were performed by measuring GFP+ area. Data was analyzed by comparing mean GFP+ area between relevant conditions using one-way ANOVA with Holm-Sidak multiple comparison test. Experiments were performed in two independent trials using a minimum of 6-8 donors except as otherwise indicated. IC50 values were calculated by fitting data to sigmoidal dose-response curve using Prism version 9.2.0 (Graphpad).

Analysis of mouse xenograft tumor models was performed by unpaired t test of targeted therapy cohorts versus combination therapy cohorts on the last day of tumor measurements using Prism version 9.2.0 (Graphpad). For dual-flank model of 3LL ΔNRAS, statistical analysis was performed by comparing median tumor volume by paired t test using Prism version 9.2.0 (Graphpad).

For all experiments, p<0.05 was considered statistically significant.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

What is claimed is:

1. A method of identifying an agent that modulates macrophage function, the method comprising:
providing a population of macrophages and a population of target cells in an assay mixture;
adding a candidate agent to the assay mixture;
culturing the assay mixture;
determining the amount of growth of the population of target cells in the assay mixture at one or more timepoints; and
comparing the amount of growth of the population of target cells in the assay mixture with the amount of growth of a population of the same type of target cells in a control mixture cultured without the candidate agent at the same timepoint;
wherein the candidate agent is determined to be an agent that modulates macrophage function when the amount of growth of the population of target cells in the assay mixture is different from the amount of growth of the population of target cells in the control mixture at the same timepoint.

2. The method of claim 1, further comprising activating the population of macrophages.

3. The method of claim 1 or 2, further comprising activating the population of macrophages by adding a macrophage-directed immunotherapy to the assay mixture prior to adding the candidate agent to the assay mixture.

4. The method of claim 3, wherein the macrophage-directed immunotherapy is a macrophage immune checkpoint inhibitor.

5. The method of claim 3 or 4, wherein the macrophage-directed immunotherapy is a CD47 inhibitor, a SIRPα inhibitor, an MHC I inhibitor, a B2M inhibitor, a CD24 inhibitor, a CALR inhibitor, a CD40 agonist, a PD-L1 inhibitor, an APMAP inhibitor, a GPR84 inhibitor, a VCAM1 inhibitor, a CD11b inhibitor, a SIGLEC-10 inhibitor, a PD-L2 inhibitor, a PD-1 inhibitor, a CD73 inhibitor, a Galectin-9 inhibitor, a CD14 inhibitor, a CD80 inhibitor, a CD86 inhibitor, a SIRPb inhibitor, a SIRPg inhibitor, a SLAMF7 inhibitor, a MARCO inhibitor, an AXL inhibitor, a CLEVER-1 inhibitor, an ILT4 inhibitor, a TIM-3 inhibitor, a TIM-4 inhibitor, an LRP-1 inhibitor, a calreticulin inhibitor, a TREM1 inhibitor, a TREM2 inhibitor, a GD2 inhibitor, an FcgRI inhibitor, an FcgRIIa inhibitor, an FcgRIIb inhibitor, an FcgRIII inhibitor, a MUC1 inhibitor, a CD44 inhibitor, a CD63 inhibitor, a CD36 inhibitor, a CD84 inhibitor, a CD164 inhibitor, a CD82 inhibitor, a CD18 inhibitor, a SIGLEC-7 inhibitor, a CD166 inhibitor, a CD39 inhibitor, a CD46 inhibitor, an LILRA1 inhibitor, an LILRA2 inhibitor, an LILRA3 inhibitor, an LILRA4 inhibitor, an LILRB1 inhibitor, an LILRB2 inhibitor, an LILRB3 inhibitor, an LILRB4 inhibitor, an LILRB5 inhibitor, a CD85b inhibitor, a CD85m inhibitor, a CD85f inhibitor, a CD276 inhibitor, a CD88 inhibitor, a CD99 inhibitor, a PILRa inhibitor, a Siglec-9 inhibitor, a CD206 inhibitor, a CD163 inhibitor, a CD84 inhibitor, a C3aR inhibitor, or a CLEC12A inhibitor.

6. The method of any of claims 3-5, wherein the macrophage-directed immunotherapy is a CD47 inhibitor or a SIRPα inhibitor.

7. The method of any of claims 3-6, wherein the macrophage-directed immunotherapy is an antibody or antibody fragment.

8. The method of any of claims 3-7, wherein the macrophage-directed immunotherapy is an anti-CD47 antibody, an anti-SIRPα antibody, a SIRPα-Fc fusion protein, an anti-MHC I antibody, an anti-CD24 antibody, an anti-CALR antibody, an anti-CD40 antibody, an anti-PD-L1 antibody, an anti-APMAP antibody, an anti-GPR84 antibody, an anti-VCAM1 antibody, an anti-CD11b antibody, an anti-SIGLEC-10 antibody, an anti-PD-L2 antibody, an anti-PD-1 antibody, an anti-B2M antibody, an anti-CD73 antibody, an anti-Galectin-9 antibody, an anti-CD14 antibody, an anti-CD80 antibody, an anti-CD86 antibody, an anti-SIRPb antibody, an anti-SIRPg antibody, an anti-SLAMF7 antibody, an anti-MARCO antibody, an anti-AXL antibody, an anti-CLEVER-1 antibody, an anti-ILT4 antibody, an anti-TIM-3 antibody, an anti-TIM-4 antibody, an anti-LRP-1 antibody, an anti-calreticulin antibody, an anti-TREM1 antibody, an anti-TREM2 antibody, an anti-GD2 antibody, an anti-FcgRI antibody, an anti-FcgRIIa antibody, an anti-FcgRIIb antibody, an anti-FcgRIII antibody, an anti-MUCI antibody, an anti-CD44 antibody, an anti-CD63 antibody, an anti-CD36 antibody, an anti-CD84 antibody, an anti-CD164 antibody, an anti-CD82 antibody, an anti-CD18 antibody, an anti-SIGLEC-7 antibody, an anti-CD166 antibody, an anti-CD39 antibody, an anti-CD46 antibody, an anti-LILRA1 antibody, an anti-LILRA2 antibody, an anti-LILRA3 antibody, an anti-LILRA4 antibody, an anti-LILRB1 antibody, an anti-LILRB2 antibody, an anti-LILRB3 antibody, an anti-LILRB4 antibody, an anti-LILRB5 antibody, an anti-CD85b antibody, an anti-CD85m antibody, an anti-CD85f antibody, an anti-CD276 antibody, an anti-CD88 antibody, an anti-CD99 antibody, an anti-PILRa antibody, an anti-Siglec-9 antibody, an anti-CD206 antibody, an anti-CD163 antibody, an anti-CD84 antibody. an anti-C3aR antibody, or an anti-CLEC12A antibody.

9. The method of any of claims 3-8, wherein the macrophage-directed immunotherapy is an anti-CD47 antibody.

10. The method of any of claims 1-9, wherein the population of macrophages comprises primary human macrophages.

11. The method of any of claims 1-10, wherein the population of macrophages comprises primary human tumor associated macrophages.

12. The method of any of claims 1-9, wherein the population of macrophages comprises macrophages derived from stem cells.

13. The method of any of claims 1-12, wherein the population of macrophages comprises at least 5,000 macrophages.

14. The method of any of claims 1-13, wherein the population of macrophages comprises at least 5,000 to about 20,000 macrophages.

15. The method of any of claims 1-14, wherein the population of macrophages comprises about 10,000 macrophages.

16. The method of any of claims 1-15, wherein the target cells are mammalian cells.

17. The method of any of claims 1-16, wherein the target cells are human cells.

18. The method of any of claims 1-17, wherein the target cells are cancer cells.

19. The method of any of claims 1-18, wherein the target cells are cancer cells resistant to an FDA-approved therapy.

20. The method of any of claims 1-19, wherein the target cells are bladder cancer cells, cervical cancer cells, dermatofibrosarcoma protuberans cells, endocrine tumor cells, neuroendocrine tumor cells, neuroblastoma cells, lung cancer cells, glioblastoma multiforme cells, bile duct cancer cells, ovarian cancer cells, stomach cancer cells, colon cancer cells, rectal cancer cells, melanoma cells, colorectal cancer cells, brain cancer cells, head and neck cancer cells, thyroid cancer cells, soft tissue cancer cells, kidney cancer cells, liver cancer cells, gastric cancer cells, gastrointestinal stromal tumor cells, giant cell tumor cells, esophageal cancer cells, gastroesophageal cancer cells, breast cancer cells, prostate cancer cells, endometrial cancer cells, pancreatic cancer cells, leukemia cells, lymphoma, cells multiple myeloma cells, colon adenocarcinoma cells, lung adenocarcinoma cells, cutaneous melanoma cells, gastrointestinal cancer cells, anal cancer cells, glioblastoma cells, laryngeal cancer cells, oral cancer cells, myelodysplastic disorder cells, myeloproliferative disorder cells, ovarian epithelial cancer cells, fallopian tube cancer cells, primary peritoneal cancer cells, plexiform neurofibroma cells, skin cancer cells, or soft tissue sarcoma cells.

21. The method of any of claims 1-20, wherein the target cells are lung cancer cells or colon cancer cells.

22. The method of any of claims 1-21, wherein the target cells express a reporter protein.

23. The method of any of claims 1-22, wherein the target cells express a reporter protein that emits light.

24. The method of any of claims 1-23, wherein the target cells express green fluorescent protein.

25. The method of any of claims 1-24, wherein determining the amount of growth of the population of target cells at a timepoint comprises automated microscopy and/or image analysis to quantify the growth of the population of target cells.

26. The method of claim 25, wherein the automated microscopy and/or image analysis comprises detecting light emission from target cells expressing a reporter protein that emits light.

27. The method of claim 25 or 26, wherein the automated microscopy and/or image analysis comprises detecting light emission over the entirety of the assay mixture.

28. The method of any of claims 1-27, wherein culturing the assay mixture takes at least 24 hours.

29. The method of any of claims 1-28, wherein culturing the assay mixture takes at least 6 days.

30. The method of any of claims 1-29, wherein culturing the assay mixture takes about 7 days.

31. The method of any of claims 1-30, wherein the timepoint for determining and comparing the amount of growth of the population of target cells with the control mixture is at least 24 hours after beginning culturing the assay mixture.

32. The method of any of claims 1-31, wherein the timepoint for determining and comparing the amount of growth of the population of target cells with the control mixture is at least 6 days after beginning culturing the assay mixture.

33. The method of any of claims 1-32, wherein the timepoint for determining and comparing the amount of growth of the population of target cells with the control mixture is about 7 days after beginning culturing the assay mixture.

34. The method of any of claims 1-33, wherein the timepoint for determining and comparing the amount of growth of the population of target cells with the control mixture is immediately after completing culturing the assay mixture.

35. The method of any of claims 1-34, wherein the agent is a small molecule.

36. The method of any of claims 1-34, wherein the agent is a biologic.

37. The method of any of claim 1-34 or 36, wherein the agent is a polypeptide, a cytokine, or an antibody or fragment thereof.

38. The method of any of claims 1-37, wherein the candidate agent is a member of a library of candidate agents.

39. The method of any of claims 1-38, wherein the agent is an activator of macrophage function such that the amount of growth of the population of target cells in the assay mixture is less than the amount of growth of the population of target cells in the control mixture.

40. The method of claim 39, wherein the population of target cells in the assay mixture at the timepoint for determining the amount of growth of the population of target cells is less than the population of target cells in the assay mixture at the time of adding the candidate agent to the mixture.

41. The method of claim 1, wherein the agent that modulates macrophage function is an inhibitor of macrophage function such that the amount of growth of the population of target cells in the assay mixture is greater than the amount of growth of the population of target cells in the control mixture.

42. The method of claim 41, wherein the population of target cells in the assay mixture at the timepoint for determining the amount of growth of the population of target cells is greater than the population of target cells in the assay mixture at the time of adding the candidate agent to the mixture.

43. The method of any of claims 1-42, further comprising providing, in the assay mixture, a population of second target cells different than the population of target cells;
determining the amount of growth of the population of second target cells in the assay mixture at one or more timepoints; and
comparing the amount of growth of the second population of target cells in the assay mixture with the amount of growth of a population of second target cells in a control mixture cultured without the candidate agent at the same timepoint;
wherein the candidate agent is determined to be an agent that promotes macrophage-mediated cytotoxicity when the amount of growth of the population of second target cells in the assay mixture is less than the amount of growth of the population of second target cells in the control mixture at the same timepoint.

44. A method of identifying an agent that promotes macrophage-mediated cytotoxicity, the method comprising:
providing a population of macrophages and a population of target cells in an assay mixture;
adding a candidate agent to the assay mixture;
culturing the assay mixture;
determining the amount of growth of the population of target cells in the assay mixture at one or more timepoints; and
comparing the amount of growth of the population of target cells in the assay mixture with the amount of growth of a population of the same type of target cells in a control mixture cultured without the candidate agent, at the same timepoint;
wherein the candidate agent is determined to be an agent that promotes macrophage-mediated cytotoxicity when the amount of growth of the population of target cells in the assay mixture is less than the amount of growth of the population of target cells in the control mixture at the same timepoint.

45. A method of identifying an agent that promotes macrophage anti-cancer function, the method comprising:
providing a population of macrophages and a population of target cells in an assay mixture;
adding a candidate agent to the assay mixture;
culturing the assay mixture:
determining the amount of growth of the population of target cells in the assay mixture at one or more timepoints; and
comparing the amount of growth of the population of target cells in the assay mixture with the amount of growth of a population of the same type of target cells in a control mixture cultured without the candidate agent at the same timepoint;
wherein the candidate agent is determined to be an agent that promotes macrophage anti-cancer function when the amount of growth of the population of target cancer cells in the assay mixture is lower than the amount of growth of the population of target cancer cells in the control mixture at the same timepoint.

46. A method of identifying a cancer therapy, the method comprising:
providing a population of macrophages and a population of target cells in an assay mixture;
adding a candidate agent to the assay mixture;
culturing the assay mixture;
determining the amount of growth of the population of target cells in the assay mixture at one or more timepoints; and
comparing the amount of growth of the population of target cells in the assay mixture with the amount of growth of a population of the same type of target cells in a control mixture cultured without the candidate agent at the same timepoint;
wherein the candidate agent is determined to be a cancer therapy when the amount of growth of the population of target cancer cells in the assay mixture is lower than the amount of growth of the population of target cancer cells in the control mixture at the same timepoint.

47. A method of identifying an agent that selectively modulates macrophage function for a target cell, the method comprising:
providing a population of macrophages, a population of first target cells, and a population of second target cells in an assay mixture, wherein the first and second target cells are different;
adding a candidate agent to the assay mixture;
culturing the assay mixture:
determining the amount of growth of the populations of first and second target cells in the assay mixture at one or more timepoints;
comparing the amount of growth of the population of first target cells in the assay mixture with the amount of growth of a population of the first target cells in a control mixture cultured without the candidate agent, at the same timepoint;
comparing the amount of growth of the population of second target cells in the assay mixture with the amount of growth of a population of the second target cells in a control mixture cultured without the candidate agent, at the same timepoint;
wherein the candidate agent is determined to be an agent that selectively modulates macrophage function for a target cell when the amount of growth of the population of first target cells in the assay mixture compared to the amount of growth in the control mixture is different than the amount of growth of the population of second target cells in the assay mixture compared to the amount of growth in the control mixture at the same timepoint.

48. An agent identified by the method of any of claims 1-47.
